(19) United States
(12) Patent Application Publication
(10) Pub. No.: US 2013/0064788 A1
(43) Pub. Date: Mar. 14, 2013

(54) IL-17 FAMILY CYTOKINE COMPOSITIONS AND USES

(71) Applicants: Thomas M. Barnes, Cambridge, MA (US); Michael M. Schmidt, Cambridge, MA (US); Bracken M. King, Cambridge, MA (US); K. Christopher Garcia, Menlo Park, CA (US); Sashank Reddy, Boston, MA (US); Gregory James Sieczkiewicz, Hopkinton, MA (US); Lauren K. Ely, Palo Alto, CA (US)

(72) Inventors: Thomas M. Barnes, Cambridge, MA (US); Michael M. Schmidt, Cambridge, MA (US); Bracken M. King, Cambridge, MA (US); K. Christopher Garcia, Menlo Park, CA (US); Sashank Reddy, Boston, MA (US); Gregory James Sieczkiewicz, Hopkinton, MA (US); Lauren K. Ely, Palo Alto, CA (US)

(73) Assignees: Eleven Biotherapeutics, Inc., Cambridge, MA (US); The Board of Trustees of the Leland Stanford Junior University, Palo Alto, CA (US)

(21) Appl. No.: 13/501,244

(22) PCT Filed: Oct. 11, 2010
(86) PCT No.: PCT/US10/52194

§ 371 Date: Nov. 30, 2012

Related U.S. Application Data
(60) Provisional application No. 61/278,779, filed on Oct. 10, 2009.

(51) Int. Cl. A61K 38/20; C12P 21/02; C12N 5/10; C12N 1/21; A61P 29/00; C12N 1/19; C07K 14/54; C07K 16/24; C07H 21/04; A61P 37/02; A61K 39/395; C12N 1/15

(52) U.S. Cl. 424/85.2; 424/139.1; 435/69.1; 435/69.52; 435/69.6; 435/325; 435/348; 435/366; 435/419; 435/252.31; 435/252.33; 435/252.34; 435/254.11; 435/254.2; 435/254.21; 435/254.23; 530/351; 530/387.9; 536/23.4; 536/23.5; 536/23.53

(57) ABSTRACT

Binding proteins, including non-naturally occurring and recombinantly modified proteins that bind to an IL-17R and including proteins having a mutated IL-17 cytokine sequence, methods of making such molecules and methods of using such molecules as therapeutic, prophylactic and diagnostic agents are provided.

### CLAIM OF PRIORITY

This application claims priority to U.S. provisional patent application Ser. No. 61/278,779, filed Oct. 10, 2009, the contents of which are incorporated in their entirety.

### GOVERNMENT FUNDING

This invention was made with Government support under (AI51321) awarded by National Institutes of Health. The Government has certain rights in this invention.

### FIELD OF INVENTION

The field of the invention is protein biochemistry and immunology. More particularly, the field relates to modified immunomodulatory polypeptides.

### BACKGROUND

The immune system protects individuals from infectious agents (e.g. viruses, bacteria, and multi-cellular organisms), as well as from cancer and neoplasms. The immune system includes many lymphoid and myeloid cell types such as neutrophils, monocytes, macrophages, dendritic cells (DCs), eosinophils, T cells, and B cells. These cells are capable of producing signaling proteins known as cytokines Cytokines are soluble, small proteins that mediate a variety of biological effects, including the induction of immune cell proliferation, development, differentiation, and/or migration, as well as the regulation of the growth and differentiation of many cell types (see, for example, Arai et al., Annu Rev. Biochem. 5P:783 (1990); Mosmann, Curr. Opin. Immunol 5:311 (1991); Paul and Seder, Cell 76:241 (1994)). Cytokine-induced immune functions can also include an inflammatory response, characterized by a systemic or local accumulation of immune cells. Although they do have host-protective effects, these immune responses can produce pathological consequences when the response involves excessive and/or chronic inflammation, as in autoimmune disorders (such as multiple sclerosis) and cancer/neoplastic diseases (Oppenheim and Feldmann (eds.) Cytokine Reference, Academic Press, San Diego, Calif. (2001); von Andrian and Mackay New Engl. J. Med. 343: 1020 (2000); Davidson and Diamond, New Engl. J. Med. 345:340 (2001); Lu et al, Mol. Cancer. Res. 4:221 (2006); Dagleish and O'Byrne, Cancer Treat Res. 130:1 (2006)).

Proteins that constitute the cytokine group include interleukins, interferons, colony stimulating factors, tumor necrosis factors, and other regulatory molecules. For example, human interleukin-17 is involved in inducing and mediating proinflammatory responses. IL-17 is commonly associated with allergic responses. IL-17 induces the production of many other cytokines (such as IL-6, G-CSF, GM-CSF, IL-1β, TGF-β, TNF-α), chemokines (including IL-8, GRO-α and MCP-1) and prostaglandins (e.g. PGE₂) from many cell types (fibroblasts, endothelial cells, epithelial cells, keratinocytes and macrophages). An abundance of evidence in recent years implicates Th17 cells as central players in the pathogenesis of numerous autoimmune and inflammatory conditions.

Accordingly, the demonstrated in vivo activities of cytokines and their receptors illustrate the clinical potential of, and need for, other cytokines, cytokine receptors, cytokine agonists, and cytokine antagonists. For example, demonstrated in vivo activities of the proinflammatory cytokine family illustrate the enormous clinical potential of, and need for antagonists of pro-inflammatory molecules such as IL-17 and IL-23.

There is an ongoing need for new compositions useful in the prevention and treatment of diseases and disorders in mammals.

### SUMMARY OF THE INVENTION

Provided are compositions and methods directed to cytokine reengineering.

In one aspect, this disclosure features an isolated antibody (including full length antibodies, antibody fragments and domains) that specifically binds to an IL-17 cytokine polypeptide, e.g., by binding to one or more of: about amino acids 21-41, 42-78, 82-103, or 104-133 of IL-17F; about amino acids 21-39, 40-76, 80-101, or 102-131 of IL-17A; about amino acids 44-65, 78-117, 121-143, or 153-179 of IL-17C; about amino acids 32-53, 66-105, 110-131, or 134-163 of IL-17D; about amino acids 27-49, 50-87, 93-114, and/or 120-148 of IL-17E; or about amino acids 32-53, 66-105, 110-131, or 135-158 of IL-17B according to the numbering in FIG. 4D.

In one embodiment, the antibody binds to an epitope in Region 1 of the IL-17 cytokine, where Region 1 corresponds to about amino acids 21 to 41 of IL-17F, about amino acids 21-39 of IL-17A, about amino acids 44-65 of IL-17C, about amino acids 32-53 of IL-17D, about amino acids 27-49 of IL-17E, or about amino acids 32-53 of IL-17B according to the numbering in FIG. 4D.

In one embodiment, the antibody binds to an epitope in Region 2 of the IL-17 cytokine, where Region 2 corresponds to about amino acids 42-78 of IL-17F, about amino acids 40-76 of IL-17A, about amino acids 78-117 of IL-17C, about amino acids 66-105 of IL-17D, about amino acids 50-87 of IL-17E, or about amino acids 66-105 of IL-17B according to the numbering in FIG. 4D.

In one embodiment, the antibody binds to an epitope in Region 3 of the IL-17 cytokine, where Region 3 corresponds to about amino acids 82-103 of IL-17F, about amino acids 80-101 of IL-17A, about amino acids 121-143 of IL-17C, about amino acids 110-131 of IL-17D, about amino acids 93-114 of IL-17E, or about amino acids 110-131 of IL-17B according to the numbering in FIG. 4D.

In one embodiment, the antibody binds to an epitope in Region 4 of the IL-17 cytokine, where Region 4 corresponds to about amino acids 104-133 of IL-17F, about amino acids 102-131 of IL-17A, about amino acids 153-179 of IL-17C, about amino acids 134-163 of IL-17D, about amino acids 120-148 of IL-17E, or about amino acids 135-158 of IL-17B according to the numbering in FIG. 4D.

In one aspect, this disclosure features an isolated antibody (including full length antibodies, antibody fragments and domains) that specifically binds to amino acids 22-36, amino acids 83-96, amino acids 118-147, amino acids 152-179, or amino acids 256-271 of IL-17RA (SEQ ID NO:14).

In another aspect, this disclosure features an isolated antibody (including full length antibodies, antibody fragments and domains) that specifically binds to amino acids 25-39, amino acids 86-100, amino acids 126-155, amino acids 160-187, or amino acids 254-269 of IL-17RB (SEQ ID NO:15) and/or amino acids 32-44 (e.g., 38-44), 82-98 (e.g., 88-98), and 252-269 (e.g., 256-263) of SEQ ID NO:15.

In another aspect, this disclosure features an isolated antibody (including full length antibodies, antibody fragments and domains) that specifically binds to amino acids 15-30, amino acids 70-84, amino acids 96-124, amino acids 129-156, or amino acids 227-237 of IL-17RC (SEQ ID NO:16) and/or amino acids 24-35, 78-91, and 248-257 of SEQ ID NO:16.

This disclosure also features:
an isolated Interleukin-17F (IL-17F) polypeptide wherein one or more of amino acids selected from the group consisting of about 21 to 41, 42-78, 82-103, and 104-133 of SEQ ID NO:12 are mutated to any other amino acid or are deleted, and for example wherein the polypeptide includes a sequence at least 90, 92, 94, 95, 96, 97, or 98% identical, but not 100% identical to SEQ ID NO:12;
an isolated Interleukin-17A (IL-17A) polypeptide wherein one or more of amino acids selected from the group consisting of about 21-39, 40-76, 80-101, and 102-131 of SEQ ID NO:2 are mutated to any other amino acid or are deleted and for example wherein the polypeptide includes a sequence at least 90, 92, 94, 95, 96, 97, or 98% identical, but not 100% identical to SEQ ID NO:2;
an isolated Interleukin-17B (IL-17B) polypeptide wherein one or more of amino acids selected from the group consisting of 32-53, 66-105, 110-131, and 135-158 of SEQ ID NO:4 are mutated to any other amino acid or are deleted and for example wherein the polypeptide includes a sequence at least 90, 92, 94, 95, 96, 97, or 98% identical, but not 100% identical to SEQ ID NO:4;
an isolated Interleukin-17C (IL-17C) polypeptide wherein one or more of amino acids selected from the group consisting of about 44-65, 78-117, 121-143, and 153-179 of SEQ ID NO:6 are mutated to any other amino acid or are deleted and for example wherein the polypeptide includes a sequence at least 90, 92, 94, 95, 96, 97, or 98% identical, but not 100% identical to SEQ ID NO:6;
an isolated Interleukin-17D (IL-17D) polypeptide wherein one or more of amino acids selected from the group consisting of 32-53, 66-105, 110-131, and 134-163 of SEQ ID NO:8 are mutated to any other amino acid or are deleted and for example wherein the polypeptide includes a sequence at least 90, 92, 94, 95, 96, 97, or 98% identical, but not 100% identical to SEQ ID NO:8;
an isolated Interleukin-17E (IL-17E) polypeptide wherein one or more of amino acids selected from the group consisting of 27-49, 50-87, 93-114, and 120-148 of SEQ ID NO:10 are mutated to any other amino acid or are deleted and for example wherein the polypeptide includes a sequence at least 90, 92, 94, 95, 96, 97, or 98% identical, but not 100% identical to SEQ ID NO:10.

The polypeptide can include additional features, including N- and C-terminal sequences, such as tags and immunoglobulin constant domains.

In another aspect this disclosure features a composition including a first and second IL-17 polypeptide, wherein at least one of the first and second polypeptide is a modified IL-17 polypeptide (e.g., a mutated IL-17 polypeptide). For example, the composition includes a first modified IL-17 polypeptide (e.g., mutated) operably linked to a second IL-17 polypeptide. In one embodiment the second IL-17 polypeptide is also a modified (e.g., mutated) IL-17 polypeptide. In another embodiment, the second IL-17 polypeptide is identical to a naturally occurring IL-17 polypeptide (e.g., a mature, human IL-17, e.g., IL-17A, IL-17B, IL-17C, IL-17D, IL-17E, and IL-17F).

The first and second polypeptides can interact to form a structure corresponding to an IL-17 dimer (e.g., a single chain dimer). The first polypeptide can be located N-terminal to the second polypeptide, or vice versa. The polypeptide chain can also include other elements; e.g., it can be a fusion protein. One or both the polypeptides can be modified, e.g., mutated relative to a reference IL-17 polypeptide (such as a human IL-17, e.g., IL-17A, IL-17B, IL-17C, IL-17D, IL-17E, and IL-17F). In one embodiment, the first and second polypeptide are components of the same polypeptide chain. In one embodiment, the first and second polypeptides are operably linked by a coiled-coil domain or a leucine zipper.

In one embodiment, the first polypeptide includes a modified IL-17A polypeptide (e.g., a mutated human IL-17A which is, e.g., at least 85, 90, 95, or 98% identical to SEQ ID NO:2 or 20), and the second polypeptide includes a modified IL-17 polypeptide selected from the group consisting of IL-17A, IL-17B, IL-17C, IL-17D, IL-17E, and IL-17F (e.g., a mutated human IL-17 cytokine which is, e.g., at least 85, 90, 95, or 98% identical to the natural mature forms of such cytokines, e.g., as disclosed herein) or a polypeptide 100% identical to a natural mature form (e.g., SEQ ID NO:2, 4, 6, 8, 10, 12, or 20). Accordingly, exemplary compositions include polypeptides corresponding to A/A homodimer, or an A/F heterodimer.

In one embodiment, the first polypeptide includes a modified IL-17F polypeptide (e.g., a mutated human IL-17F which is, e.g., at least 85, 90, 95, or 98% identical to SEQ ID NO:12), and the second polypeptide includes a modified IL-17 polypeptide selected from the group consisting of IL-17A, IL-17B, IL-17C, IL-17D, IL-17E, and IL-17F (e.g., a mutated human IL-17 cytokine which is, e.g., at least 85, 90, 95, or 98% identical to the natural mature forms of such cytokines, e.g., as disclosed herein) or a polypeptide 100% identical to a natural mature form (e.g., SEQ ID NO:2, 4, 6, 8, 10, 12, or 20). Accordingly, exemplary compositions include polypeptides corresponding to F/F homodimer, or an A/F heterodimer.

In one embodiment, the first polypeptide includes a modified IL-17 cytokine polypeptide (e.g., a mutated human IL-17B, IL-17C, IL-17D, or IL-17E which is, e.g., at least 85, 90, 95% identical to SEQ ID NO:4, 6, 8, or 10), and the second polypeptide includes a modified IL-17 polypeptide selected from the group consisting of IL-17A, IL-17B, IL-17C, IL-17D, IL-17E, and IL-17F (e.g., a mutated human IL-17 cytokine which is, e.g., at least 85, 90, 95% identical to the natural mature forms of such cytokines, e.g., as disclosed herein). Accordingly, exemplary compositions include polypeptides corresponding to B/B, C/C, D/D, E/E homodimer, and various heterodimers.

In another aspect, the disclosure features a composition that includes an isolated polypeptide including an IL-17 binding determinant of IL-17RA, wherein the polypeptide is not identical to the extracellular domain of IL-17RA. For example, the IL-17 binding determinant is selected from the group consisting of amino acids 22-36, 83-96, 118-147, 152-179, and 256-271 of IL-17RA (SEQ ID NO:14). The binding determinant can be a peptide, e.g., a peptide that includes or consists of amino acids 22-36, 83-96, 118-147, 152-179, and 256-271 of IL-17RA. The binding determinant can be, e.g., an IL-17F, IL-17A, or IL-17C binding determinant. In one embodiment, the polypeptide is capable of binding IL-17F and/or IL-17A. Binding of the polypeptide to IL-17A can include contacts with one or more amino acids selected from the group consisting of about 21-39, 40-76, 80-101, and 102-131 of the IL-17A. Binding of the polypeptide to IL-17C can include contacts with one or more amino acids selected from the group consisting of about 44-65, 78-117, 121-143, and 153-179 of the IL-17C.

In some embodiments, the polypeptide is capable of forming a cysteine knot motif or a four-helix bundle motif. In one embodiment, the polypeptide is operably bound to an IL-17RA polypeptide, e.g., an extracellular region of an IL-17RA polypeptide, and an IL-17RC polypeptide, e.g., an extracellular region of an IL-17RC polypeptide.

In another aspect, the disclosure features a composition that includes an isolated polypeptide including an IL-17 binding determinant of IL-17RC, wherein the polypeptide is not identical to the extracellular domain of IL-17RC. The polypeptide can be operably bound to a binding partner selected from an IL-17RA polypeptide, e.g., an extracellular region of an IL-17RA polypeptide, and an IL-17RC polypeptide e.g., an extracellular region of an IL-17RC polypeptide. The polypeptide can bind to an IL-17 cytokine, e.g., to IL-17A and contact one or more amino acids selected from the group consisting of about 21-39, 40-76, 80-101, and 102-131 of the IL-17A, or IL-17C and contact one or more of amino acids selected from the group consisting of about 44-65, 78-117, 121-143, and 153-179 of the IL-17C.

In another aspect, the disclosure features an IL-17R binding protein that includes a first and second IL-17 subunit and wherein the subunits form a dimer comprising a first face that is able to interact with a first IL-17 receptor subunit and a second face that has reduced or no ability to interact with a second IL-17 receptor subunit relative to a corresponding natural IL-17 protein. For example, the first and second subunits differ from one another. Each subunit can be at least 85, 87, 90, 92, 94, 95, 96, 97, or 98% identical to a mature IL-17 cytokine, e.g., a human IL-17 cytokine (SEQ ID NO:2, 4, 6, 8, 10, 12, or 20) or a murine IL-17 cytokine, e.g., the same reference cytokine for both subunits or different reference cytokines (e.g., an IL-17A and IL-17F).

In certain embodiments, each subunit is at least 85, 87, 90, 92, 94, 95, 96, 97, or 98% identical to a mature IL-17 cytokine in region corresponding to 1-127 of SEQ ID NO:127 or 1-126 of SEQ ID NO:20.

In one embodiment, each subunit has one, two, three, four, five, six, seven or more substitutions or deletions relative to a mature human IL-17 cytokine (SEQ ID NO:2, 4, 6, 8, 10, 12, or 20), preferably fewer than twelve, ten, nine, eight, seven, six, or five. In one embodiment, one subunit has between one and five, seven, or eight mutations relative to a mature human IL-17 cytokine (SEQ ID NO:2, 4, 6, 8, 10, 12, or 20) and the other subunit has a C-terminal deletion of at least one, two, three, four or five amino acids and optionally between one and five substitutions relative to a mature human IL-17 cytokine (SEQ ID NO:2, 4, 6, 8, 10, 12, or 20).

In one embodiment, the second face of the dimer comprises at least one, two, or three mutations, e.g., at least one, two, or three substitutions. For example, the second face of the dimer has one, two, three, four, five, six, seven, eight, nine, ten, eleven, or twelve mutations (e.g., substitutions). The mutations can be located in one or more sites. The first face can be such that it does not contain any mutations relative to a mature IL-17 cytokine, e.g., a mature human IL-17 cytokine (SEQ ID NO:2, 4, 6, 8, 10, 12, or 20).

In one embodiment, the second face of the dimer comprises at least one, two, or three mutations in Site 1, e.g., at least one, two, or three substitutions. For example, the second face of the dimer has between one and three, four, five, or six mutations (e.g., substitutions) in Site 1.

In one embodiment, the second face of the dimer comprises at least one, two, or three mutations in Site 2, e.g., at least one, two, or three substitutions. For example, the second face of the dimer has between one and three, four, five, or six mutations (e.g., substitutions) in Site 2.

In one embodiment, the second face of the dimer comprises at least one, two, or three mutations in Site 3, e.g., at least one, two, or three substitutions. For example, the second face of the dimer has between one and three, four, five, or six mutations (e.g., substitutions) in Site 3.

The first and second subunits can be covalently attached, e.g., they can be components of the same polypeptide chain. For example, they can be joined by a flexible linker.

In one embodiment, the binding protein has less than 1% of the cytokine activity of IL-17A/A. For example, it does not substantially agonize IL-17 receptors, e.g., based on an assay described herein.

In one embodiment, the binding protein has an affinity for IL-17RA or IL-17RC that is no more than 100-, 50-, 20, 10-fold weaker than IL-17A/A, IL-17F/F, or IL-17A/F. Generally, the binding protein cannot bind to both IL-17RA and IL-17RC to form a complex containing the binding protein and both IL-17RA and IL-17RC. The binding protein can have other features and properties described herein.

A binding protein described herein can include two IL-17 subunits wherein each subunit is at least 85, 87, 90, 92, 94, 95, 96, 97, or 98% identical to a mature IL-17 cytokine, e.g., a human IL-17 cytokine (SEQ ID NO:2, 4, 6, 8, 10, 12, or 20) and collectively the subunits includes at least two, three, four, five, or more of the following substitutions or deletions relative to such mature IL-17 polypeptide:
in the first subunit, a substitution at the position corresponding to R47 (according to numbering in SEQ ID NO:12) (e.g., R47E, R47A, or R47D),
in the first subunit, a substitution at the position corresponding to S65 (according to numbering in SEQ ID NO:12) (e.g., S65K, S65R, or S65W)
in the first subunit, a substitution at the position corresponding to W68 (according to numbering in SEQ ID NO:12) (e.g., W68A, W68V, W68S, W68Q or W68N),
in the first subunit, a substitution at the position corresponding to R102 (according to numbering in SEQ ID NO:12) (e.g., R102A, R102V, R102S or R102T),
in the second subunit, a substitution at the position corresponding to N89 (according to numbering in SEQ ID NO:12) (e.g., N89A or N89V),
in the second subunit, a substitution at the position corresponding to Q95 (according to numbering in SEQ ID NO:12) (e.g., Q95A or Q95W), and
in the second subunit, one or more substitutions or deletions at positions corresponding to 127-132 (according to numbering in SEQ ID NO:12) (e.g., a deletion of at least positions corresponding to 128-132).

For example, the binding protein can have at least one or more of the following combinations (e.g., pairings) of mutations with respect to the positions indicated above: (R47, S65), (R47, W68), (R47, R102), (S65, W68), (S65, R102), (R47, N89), (R47, Q95), (N89, R102), (N89, deletion of 128-132), (R47, N89, R102), (N89, Q95), (W68, R102), (N89, W68), (R47, S65, N89), (R47, W68, N89), (R47, N89, R102), (R47, W68, N89, deletion of 128-132), (R47, S65, N89, deletion of 128-132), (S65, N89, deletion of 128-132), (R47, S65, deletion of 128-132), and (N89, Q95, deletion of 128-132). The binding protein can have other features and properties described herein.

Also featured are nucleic acids that include sequences encoding the polypeptides described herein, including sequences encoding one or more cytokine subunits as described herein. The nucleic acid can further include vector sequences, and transcriptional and translational control sequences. Also featured are host cells containing such nucleic acids, and methods that include expressing such nucleic acids, e.g., in a cell. The methods can further include recovering the protein, e.g., by purification from the cells or cell media.

Further features and advantages will now be more particularly described in the following detailed description and claims.

### BRIEF DESCRIPTION OF THE FIGURES

FIG. 1 is a schematic illustration of the structure of the IL-17RA-IL-17F complex. Ribbon diagram of IL-17RA in bound to IL-17F (chain A and chain B), N-linked glycans are shown in ball-and-stick representation. IL-17RA is composed of two fibronectin type III domains (D1 and D2) joined by a short helical linker. The right-hand panel shows the complex rotated by 60° around the y-axis.

FIG. 2 is a schematic illustration demonstrating IL-17F binding to IL-17RA is mediated by three distinct interfaces. (A) Site 2, the IL-17RA D1 C-C′ loop inserts between the N-terminal coil region and strands 1 and 2 of the IL-17F chain B. The N-terminal coil undergoes a conformational change between the unbound and bound conformations. (B) Site 2, surface representation of the knob-in-holes IL-17F binding pocket complementarity. (C) Site 1, the IL-17RA D1 N-terminal binding site. (D) Site 3, the IL-17RA D2 binding site. Contact residues are shown as stick models. Dotted lines represent hydrogen bonds and salt-bridges.

FIG. 3 is an assembly and model of the heterodimeric IL-17 signaling complex. (A) IL-17 receptor-cytokine affinity was measured by surface plasmon resonance (SPR). IL-17RA, IL-17RB and IL-17RC were immobilized on the SPR chip surface, and the binding affinity of IL-17A, IL-17F or IL-17E was measured. Where indicated, the affinity of a second receptor binding to the pre-assembled receptor-cytokine complex on the chip was then measured. For kinetic experiments (top 3 rows), representative SPR sensorgrams are shown as colored lines and the curve-fit as a black line. Time in seconds (s) is plotted against response (RU, resonance units). The injected concentrations are to the right of the sensorgrams. For equilibrium experiments (fourth row), the injected concentration (M) is plotted against the maximum response (RU) for a representative experiment; the curve fit is shown as a black line and the dissociation constant (Kd) is marked as a vertical line. The insets show cartoon representations of the binding event. The Kd is reported as the mean of at least two independent experiments±the standard error of the mean. (B) Model of heterodimeric signaling complex formation. The second receptor (magenta) was modeled assuming that both receptors bind to IL-17F in the same orientation. The C-terminal domains (D2) of the receptors come into close proximity as highlighted by the box.

FIG. 4 is a schematic illustration demonstrating the binding interface and conserved IL-17 residues. Surface representation of IL-17F in white with IL-17RA in ribbon format colored yellow. (A) IL-17RA-IL-17F contact residues highlighted in cyan. (B) Residues conserved among IL-17A and IL-17F are mapped onto the IL-17F structure; identical residues are stippled and conservative substitutions in light pink. (C) Residues identical among 4, 5 or 6 IL-17 cytokine family members are indicated and conservative substitutions across all six cytokines are also identified. (D) Alignment of human IL-17 cytokines Residues that form contacts in the IL-17RA-IL-17F structure are highlighted by a black box on the IL-17F sequence and underneath the alignment. Residues that are identical in four, five or six cytokines are stippled; those identical in all six cytokines are also marked with ‘*’; conserved groups are marked with ‘:’. The sequences correspond to SEQ ID NOs:12, 2, 6, 8, 10, and 4, respectively.

FIG. 5 is a comparison of the IL-17RA-IL-17F receptor complex compared to homodimeric cysteine-knot growth factor receptor complexes. (A) IL-17RA-IL-17F, (B) P75NTR-NGF and (C) TrkA-NGF are shown as ribbon models.

### DETAILED DESCRIPTION

In order for the present invention to be more readily understood, certain terms and phrases are defined below as well as throughout the specification.

### DEFINITIONS

The term “effective amount” as used herein refers to the amount necessary to elicit a desired biological response. The effective amount of a drug may vary depending on such factors as the desired biological endpoint, the drug to be delivered, the composition of any additional active or inactive ingredients, etc.

The term “expression” is used herein to mean the process by which a polypeptide is produced from DNA. The process involves the transcription of the gene into mRNA and the translation of this mRNA into a polypeptide. Depending on the context in which it is used, “expression” may refer to the production of RNA, protein, or both.

The term “gene product” as used herein means an RNA (for example, a messenger RNA (mRNA) or a micro RNA (miRNA)) or protein that is encoded by the gene.

As used herein, the term “isolated” refers to a molecule that is substantially pure. An isolated protein can be substantially pure, e.g., 60%, 65%, 70%, 75%, 80%, 85%, 90%, 95% or 99% free of other, different protein molecules.

As used herein, the terms “modulate” and “modulation” generally refer to the downregulation (i.e., inhibition or suppression), of specifically targeted genes (including their RNA and/or protein products), signaling pathways, cells, and/or a targeted phenotype, or the upregulation (i.e., induction or increase) of the targeted genes. For example, “modulate” and “modulation” can refer to downregulation of IL-17 receptor signaling.

“Patient” or “subject” means a mammal, e.g. a human, who has or is at risk for developing a disease or condition such as an inflammatory disease, or has or is diagnosed as having an inflammatory disease, or could otherwise benefit from the compositions and methods described herein.

The term “reduce” as used herein refers to any inhibition, reduction, decrease, suppression, downregulation, or prevention in expression or gene product activity. For example, the level of expression or activity can be, for example, 100% or less than 100%, for example, less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, or less than 5% of the uninhibited expression or activity.

The terms “treating” or “treatment” or “alleviation” or “amelioration” refer to both therapeutic treatment and prophylactic or preventative measures, wherein the object is to prevent or slow down (lessen) the targeted pathologic condition or disorder.

The term “IL-17 receptor” refers to proteins that bind to an IL-17 cytokine such as IL-17RA, IL-17RB, IL-17RC, IL-17RD, and IL-17RE receptors, particularly human isoforms of these receptors and extracellular domains of these receptors.

Calculations of “homology” or “sequence identity” between two sequences (the terms are used interchangeably herein) are performed as follows. The sequences are aligned for optimal comparison purposes (e.g., gaps can be introduced in one or both of a first and a second amino acid or nucleic acid sequence for optimal alignment). The optimal alignment is determined as the best score using the Needleman and Wunsch algorithm as implemented in the Needle algorithm of the EMBOSS package using a Blossum 62 scoring matrix with a gap penalty of 10, and a gap extend penalty of 1. See Needleman, S. B. and Wunsch, C. D. (1970) J. Mol. Biol. 48, 443-453; Kruskal, J. B. (1983) An overview of sequence comparison In D. Sankoff and J. B. Kruskal, (ed.), Time warps, string edits and macromolecules: the theory and practice of sequence comparison, pp. 1-44 Addison Wesley, and tools available from the European Bioinformatics Institute (Cambridge UK) EMBOSS: The European Molecular Biology Open Software Suite (2000), Rice, P. et al., A., Trends in Genetics 16, (6) pp. 276-277 and available online at hypertext transfer protocol://www.ebi.ac.uk/Tools/emboss/align/index.html and hypertext transfer protocol://emboss.open-bio.org/wiki/Appdoc:Needle. The amino acid residues or nucleotides at corresponding amino acid positions or nucleotide positions are then compared, and the percent identity between the two sequences is a function of the number of identical positions shared by the sequences.

### Immunomodulatory Polypeptides

Naïve T cells are stimulated to differentiate into specialized effector cells primarily through the actions of secreted cytokines. T helper (T_{H}) cells have been typically considered to fall into one of two effector cell lineages; T_{H}1 and T_{H}2 cells modulating cellular and humoral T cell immunity, respectively, based on their cytokine expression profiles (1). More recent work described Th17 cells, a third lineage of effector T_{H} cells distinct from, and in fact antagonized by products of the Th1 and Th2 lineages (2,3). Named after their signature cytokine interleukin 17 (IL-17), this subset of Th cells appear to have evolved as an arm of the adaptive immune system specialized for enhanced host protection against extracellular bacteria and some fungi, as these microbes may not be effectively controlled by Th1 or Th2 responses (4, 5). The varied tissue sources of cytokines that induce differentiation and regulate homeostasis of Th17 cells, namely IL-23, IL-6, and transforming growth factor-β (TGF-β), together with the presence of IL-17 receptors on both hematopoietic and non-hematopoietic cells, have highlighted the complicated relationships that exist between adaptive and innate immune cells. While the full scope of Th17 cell effector functions is still emerging, the strong inflammatory response promoted by Th17 cells has been associated with the pathogenesis of a number of autoimmune and inflammatory disorders previously attributed to Th1 or Th2 cells including rheumatoid arthritis, multiple sclerosis and psoriasis (4).

In addition to IL-17A, members of the IL-17 family include IL-17B, IL-17C, IL-17D, IL-17E (also termed IL-25), and IL-17F. All members of the IL-17 family have a similar protein structure including four highly conserved cysteine residues. IL-17A and F are most closely related followed by IL-17B (29%), IL-17D (25%), IL-17C (23%), and IL-17E being most distantly related to IL-17A (17%). These cytokines are all well conserved in mammals, with as much as 62-88% of amino acids conserved between the human and mouse homologs. There is no sequence similarity to other cytokines. On the basis of the crystal structure of IL-17F, the six structurally related IL-17 cytokines (IL-17A-IL-17F) are predicted to form a homodimeric fold (or heterodimeric fold in the case of IL-17A-F) homologous to that of the cysteine-knot growth factors such as nerve growth factor (NGF) (7, 8). Th17 cell-derived IL-17A and IL-17F share the greatest homology within the family and require both IL-17RA and IL-17RC for signaling (9, 10). While it has been shown that fibroblasts, epithelial and endothelial cells coexpress both IL-17RA and IL-17RC, T cells do not demonstrably express IL-17RC, and only express IL-17RA (11). It was thought that lymphocytes are not responsive to IL-17; however, Flavell and coworkers reported that T cells indeed can directly respond to IL-17 (12).

The IL-17 family of cytokines, in part through their actions as effector cytokines of the Th17 lineage, provides innovative approaches to the manipulation of immune and inflammatory responses. As such, antagonists of IL-17A, IL-17B, IL-17C, IL-17D, IL-17E, IL-17F, and their receptors, either singly or together, such as antagonists described herein, are useful in therapeutic treatment of inflammatory diseases such as multiple sclerosis, inflammatory bowel disease (IBD), rheumatoid arthritis, psoriasis, and cancer. Moreover, antagonists of IL-17 family member activity, such as antagonists described herein, are useful in therapeutic treatment of other inflammatory diseases.

Some exemplary sequences for human IL-17 cytokines are as follows.

IL-17A. An exemplary human IL-17A cytokine sequence is as follows and is described by UniProt identifier Q16552 (see web resources at uniprot.org and The UniProt Consortium, Nucleic Acids Res. D142-D148 (2010)):

(SEQ ID NO: 1)
MTPGKTSLVS LLLLLSLEAI VKAGITIPRN PGCPNSEDKN
FPRTVMVNLNIHNRNTNTNP KRSSDYYNRS TSPWNLHRNE
DPERYPSVIW EAKCRHLGCINADGNVDYHM NSVPIQQEIL
VLRREPPHCP NSFRLEKILV SVGCTCVTPIVHHVA

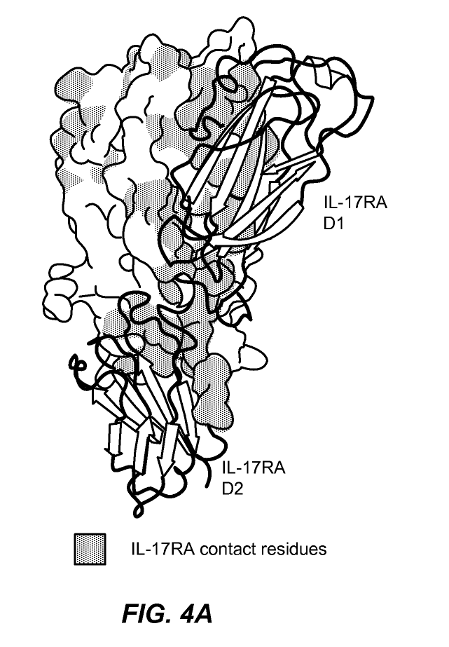

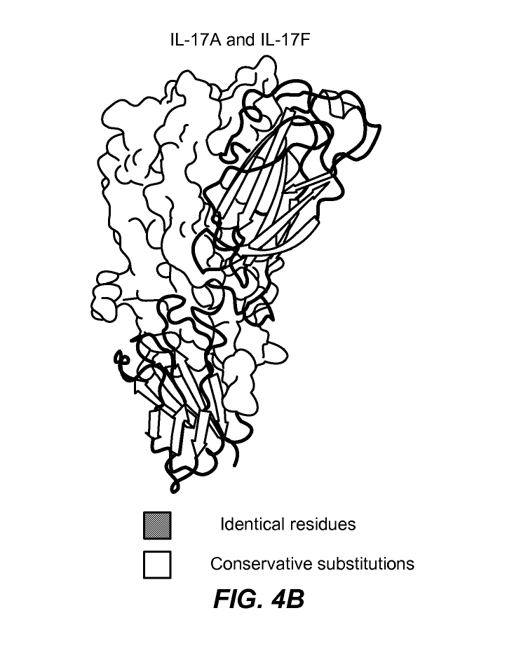

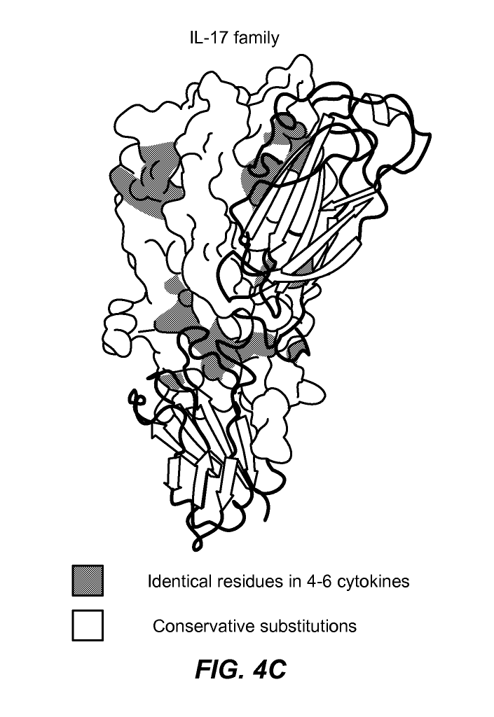

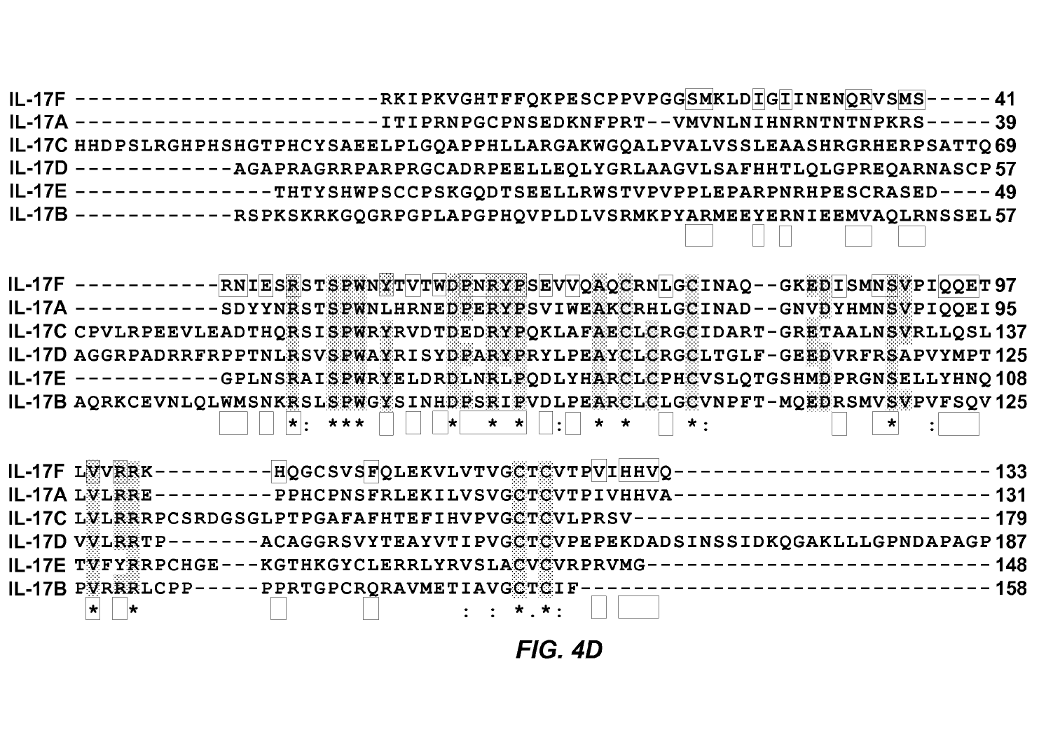

Another exemplary sequence includes amino acids 24-155 of the sequence above, forms lacking the IL-17A signal sequence, or the sequence shown in FIG. 4D:

(SEQ ID NO: 2)
ITIPRNPGCPNSEDKNFPRTVMVNLNIHNRNTNTNPKRSSDYYNRSTS
PWNLHRNEDPERYPSVIWEAKCRHLGCINADGNVDYHMNSVPIQQEIL
VLRREPPHCPNSFRLEKILVSVGCTCVTPIVHHVA.

The sequence can also include a glycine preceding the first residue of SEQ ID NO:2. Each IL-17A sequence described herein with reference to SEQ ID NO:2 can also include this glycine that precedes the isoleucine that is the first listed amino acid in SEQ ID NO:2. Other residues can also be used. Other exemplary IL-17A sequences include murine (Q62386), rat (Q61453) and bovine sequences (Q687Y7). Mutations and modifications described herein can be made in IL-17A sequences from any species, e.g., as described herein.

IL-17B. An exemplary human IL-17B cytokine sequence is as follows and is described by Uniprot identifier Q9UHF5:

(SEQ ID NO: 3)
MDWPHNLLFLLTISIFLGLGQPRSPKSKRKGQGRPGPLAPGPHQVPL
DLVSRMKPYARMEEYERNIEEMVAQLRNSSELAQRKCEVNLQLWMSN
KRSLSPWGYSINHDPSRIPVDLPEARCLCLGCVNPFTMQEDRSMVSV
PVFSQVPVRRRLCPPPPRTGPCRQRAVMETIAVGCTCIF.

Another exemplary sequence includes amino acids 21-180 of the sequence above, forms lacking the IL-17B signal sequence, or the sequence shown in FIG. 4D:

(SEQ ID NO: 4)
RSPKSKRKGQGRPGPLAPGPHQVPLDLVSRMKPYARMEEYERNIEEM
VAQLRNSSELAQRKCEVNLQLWMSNKRSLSPWGYSINHDPSRIPVDL
PEARCLCLGCVNPFTMQEDRSMVSVPVFSQVPVRRRLCPPPPRTGPC
RQRAVMETIAVGCTCIF.

IL-17C. An exemplary IL-17C cytokine sequence is as follows and is described by Uniprot identifier Q9P0M4:

(SEQ ID NO: 5)
MTLLPGLLFLTWLHTCLAHHDPSLRGHPHSHGTPHCYSAEELPLGQAP
PHLLARGAKWGQALPVALVSSLEAASHRGRHERPSATTQCPVLRPEEV
LEADTHQRSISPWRYRVDTDEDRYPQKLAFAECLCRGCIDARTGRETA
ALNSVRLLQSLLVLRRRPCSRDGSGLPTPGAFAFHTEFIHVPVGCTCV
LPRSV.

Another exemplary sequence includes amino acids 19-197 of the sequence above, forms lacking the IL-17C signal sequence, or the sequence shown in FIG. 4D:

(SEQ ID NO: 6)
HHDPSLRGHPHSHGTPHCYSAEELPLGQAPPHLLARGAKWGQALPVAL
VSSLEAASHRGRHERPSATTQCPVLRPEEVLEADTHQRSISPWRYRVD
TDEDRYPQKLAFAECLCRGCIDARTGRETAALNSVRLLQSLLVLRRRP
CSRDGSGLPTPGAFAFHTEFIHVPVGCTCVLPRSV.

IL-17D. An exemplary IL-17D cytokine sequence is as follows and is described by Uniprot Identifier Q8TAD2:

(SEQ ID NO: 7)
MLVAGFLLALPPSWAAGAPRAGRRPARPRGCADRPEELLEQLYGRLAA
GVLSAFHHTLQLGPREQARNASCPAGGRPADRRFRPPTNLRSVSPWAY
RISYDPARYPRYLPEAYCLCRGCLTGLFGEEDVRFRSAPVYMPTVVLR
RTPACAGGRSVYTEAYVTIPVGCTCVPEPEKDADSINSSIDKQGAKLL
LGPNDAPAGP.

Another exemplary sequence includes amino acids 16-202 of the sequence above, forms lacking the IL-17D signal sequence, or the sequence shown in FIG. 4D:

(SEQ ID NO: 8)
AGAPRAGRRPARPRGCADRPEELLEQLYGRLAAGVLSAFHHTLQLGPRE
QARNASCPAGGRPADRRFRPPTNLRSVSPWAYRISYDPARYPRYLPEAY
CLCRGCLTGLFGEEDVRFRSAPVYMPTVVLRRTPACAGGRSVYTEAYVT
IPVGCTCVPEPEKDADSINSSIDKQGAKLLLGPNDAPAGP.

IL-17E. An exemplary IL-17E cytokine sequence (also termed IL-25) is as follows and is described by Uniprot Identifier Q9H293:

(SEQ ID NO: 9)
MRERPRLGEDSSLISLFLQVVAFLAMVMGTHTYSHWPSCCPSKGQDTS
EELLRWSTVPVPPLEPARPNRHPESCRASEDGPLNSRAISPWRYELDR
DLNRLPQDLYHARCLCPHCVSLQTGSHMDPRGNSELLYHNQTVFYRRP
CHGEKGTHKGYCLERRLYRVSLACVCVRPRVMG.

Another exemplary sequence includes amino acids 33-177 of the sequence above, forms lacking the IL-17E signal sequence, or the sequence shown in FIG. 4D:

(SEQ ID NO: 10)
THTYSHWPSCCPSKGQDTSEELLRWSTVPVPPLEPARPNRHPESCRAS
EDGPLNSRAISPWRYELDRDLNRLPQDLYHARCLCPHCVSLQTGSHMD
PRGNSELLYHNQTVFYRRPCHGEKGTHKGYCLERRLYRVSLACVCVRP
RVMG.

IL-17F. An exemplary IL-17F cytokine sequence is as follows and is described by Uniprot Identifier Q96PD4:

(SEQ ID NO: 11)
MTVKTLHGPAMVKYLLLSILGLAFLSEAAARKIPKVGHTFFQKPESCP
PVPGGSMKLDIGIINENQRVSMSRNIESRSTSPWNYTVTWDPNRYPSE
VVQAQCRNLGCINAQGKEDISMNSVPIQQETLVVRRKHQGCSVSFQLE
KVLVTVGCTCVTPVIHHVQ.

Translation of the cytokine can initiate at MET1 or MET11 of the foregoing sequence.

Another exemplary sequence includes amino acids 31-163 of the sequence above, forms lacking the IL-17F signal sequence, or the sequence shown in FIG. 4D:

(SEQ ID NO: 12)
RKIPKVGHTFFQKPESCPPVPGGSMKLDIGIINENQRVSMSRNIESRS
TSPWNYTVTWDPNRYPSEVVQAQCRNLGCINAQGKEDISMNSVPIQQE
TLVVRRKHQGCSVSFQLEKVLVTVGCTCVTPVIHHVQ.

The sequence provides a useful default reference for identifying the position of residues in IL-17 family members in conjunction with FIG. 4D. Other exemplary IL-17F sequences include murine (Q7TNI7), rat (Q5BJ95) and porcine sequences (Q5BJ95).

The sequences of several other mammalian IL-17 cytokines are also known. See, e.g., Uniprot entries: Q62386 (murine IL-17A), Q61453 (rat IL-17A), Q687Y7 (bovine IL-17A), Q7TNI7 (murine IL-17F), Q5BJ95 (rat IL-17F), Q9QXT6 (murine IL-17B), Q9EQI6 (hamster IL-17B), Q8K4C5 (murine IL-17C), Q8K4C4 (murine IL-17D), and Q9VHH8 (murine IL-17E).

IL-17RA. An exemplary human IL-17RA receptor sequence is as follows and is described by UniProt identifier Q96F46:

(SEQ ID NO: 13)
MGAARSPPSAVPGPLLGLLLLLLGVLAPGGASLRLLDHRALVCSQPGL
NCTVKNSTCLDDSWIHPRNLTPSSPKDLQIQLHFAHTQQGDLFPVAHI
EWTLQTDASILYLEGAELSVLQLNTNERLCVRFEFLSKLRHHHRRWRF
TFSHFVVDPDQEYEVTVHHLPKPIPDGDPNHQSKNFLVPDCEHARMKV
TTPCMSSGSLWDPNITVETLEAHQLRVSFTLWNESTHYQILLTSFPHM
ENHSCFEHMHHIPAPRPEEFHQRSNVTLTLRNLKGCCRHQVQIQPFFS
SCLNDCLRHSATVSCPEMPDTPEPIPDYMPLWVYWFITGISILLVGSV
ILLIVCMTWRLAGPGSEKYSDDTKYTDGLPAADLIPPPLKPRKVWHYS
ADHPLYVDVVLKFAQFLLTACGTEVALDLLEEQAISEAGVMTWVGRQK
QEMVESNSKIIVLCSRGTRAKWQALLGRGAPVRLRCDHGKPVGDLFTA
AMNMILPDFKRPACFGTYVVCYFSEVSCDGDVPDLFGAAPRYPLMDRF
EEVYFRIQDLEMFQPGRMHRVGELSGDNYLRSPGGRQLRAALDRFRDW
QVRCPDWFECENLYSADDQDAPSLDEEVFEEPLLPPGTGIVKRAPLVR
EPGSQACLAIDPLVGEEGGAAVAKLEPHLQPRGQPAPQPLHTLVLAAE
EGALVAAVEPGPLADGAAVRLALAGEGEACPLLGSPGAGRNSVLFLPV
DPEDSPLGSSTPMASPDLLPEDVREHLEGLMLSLFEQSLSCQAQGGCS
RPAMVLTDPHTPYEEEQRQSVQSDQGYISRSSPQPPEGLTEMEEEEEE
EQDPGKPALPLSPEDLESLRSLQRQLLFRQLQKNSGWDTMGSESEGPSA

Also provided is an IL-17RA polypeptide in which the signal sequence is removed (e.g., processed) or in which amino acids 1-31 or 1-32 are deleted, and optionally other deletions, insertions and substitutions. An exemplary IL-17RA polypeptide is as follows:

(SEQ ID NO: 14)
SLRLLDHRALVCSQPGLNCTVKNSTCLDDSWIHPRNLTPSSPKDLQIQL
HFAHTQQGDLFPVAHIEWTLQTDASILYLEGAELSVLQLNTNERLCVRF
EFLSKLRHHHRRWRFTFSHFVVDPDQEYEVTVHHLPKPIPDGDPNHQSK
NFLVPDCEHARMKVTTPCMSSGSLWDPNITVETLEAHQLRVSFTLWNES
THYQILLTSFPHMENHSCFEHMHHIPAPRPEEFHQRSNVTLTLRNLKGC
CRHQVQIQPFFSSCLNDCLRHSATVSCPEMPDTPEPIPDYMPLWVYWFI
TGISILLVGSVILLIVCMTWRLAGPGSEKYSDDTKYTDGLPAADLIPPP
LKPRKVWIIYSADHPLYVDVVLKFAQFLLTACGTEVALDLLEEQAISEA
GVMTWVGRQKQEMVESNSKIIVLCSRGTRAKWQALLGRGAPVRLRCDHG
KPVGDLFTAAMNMILPDFKRPACFGTYVVCYFSEVSCDGDVPDLFGAAP
RYPLMDRFEEVYFRIQDLEMFQPGRMHRVGELSGDNYLRSPGGRQLRAA
LDRFRDWQVRCPDWFECENLYSADDQDAPSLDEEVFEEPLLPPGTGIVK
RAPLVREPGSQACLAIDPLVGEEGGAAVAKLEPHLQPRGQPAPQPLHTL
VLAAEEGALVAAVEPGPLADGAAVRLALAGEGEACPLLGSPGAGRNSVL
FLPVDPEDSPLGSSTPMASPDLLPEDVREHLEGLMLSLFEQSLSCQAQG
GCSRPAMVLTDPHTPYEEEQRQSVQSDQGYISRSSPQPPEGLTEMEEEE
EEEQDPGKPALPLSPEDLESLRSLQRQLLFRQLQKNSGWDTMGSESEGP
SA

and represents the numbering used in Examples 1-3 below.

Another exemplary IL-17RA polypeptide includes the extracellular domain of IL-17RA, e.g., about amino acids 33-320 of SEQ ID NO:13. Other exemplary IL-17RA sequences include murine (Q60943), rat (NP_{—}001101353.2, GenBank) and bovine sequences (XP_{—}603383.5, GenBank).

IL-17RB. An exemplary human IL-17RB receptor sequence is as follows and has a Q9NRM6 UniProt identifier:

(SEQ ID NO: 15)
MSLVLLSLAALCRSAVPREPTVQCGSETGPSPEWMLQHDLIPGDLRDLR
VEPVTTSVATGDYSILMNVSWVLRADASIRLLKATKICVTGKSNFQSYS
CVRCNYTEAFQTQTRPSGGKWTFSYIGFPVELNTVYFIGAHNIPNANMN
EDGPSMSVNFTSPGCLDHIMKYKKKCVKAGSLWDPNITACKKNEETVEV
NFTTTPLGNRYMALIQHSTIIGFSQVFEPHQKKQTRASVVIPVTGDSEG
ATVQLTPYFPTCGSDCIRHKGTVVLCPQTGVPFPLDNNKSKPGGWLPLL
LLSLLVATWVLVAGIYLMWRHERIKKTSFSTTTLLPPIKVLVVYPSEIC
FHHTICYFTEFLQNHCRSEVILEKWQKKKIAEMGPVQWLATQKKAADKV
VFLLSNDVNSVCDGTCGKSEGSPSENSQDLFPLAFNLFCSDLRSQIHLH
KYVVVYFREIDTKDDYNALSVCPKYHLMKDATAFCAELLHVKQQVSAGK
RSQACHDGCCSL

See also Tian et al., Oncogene 19:2098-2109 (2000) and Shi et al., J. Biol. Chem. 275:19167-19176 (2000). Also provided is an IL-17RB polypeptide in which the signal sequence is removed (e.g., processed) or in which amino acids 1-17 are deleted, and optionally other deletions, insertions and substitutions. Another exemplary IL-17RB polypeptide includes the extracellular domain of IL-17RB, e.g., about amino acids 18-292 of Q9NRM6.

IL-17RC. An exemplary human IL-17RC receptor sequence is as follows and has a Q8NAC3 UniProt identifier:

(SEQ ID NO: 16)
MPVPWFLLSLALGRSPVVLSLERLVGPQDATHCSPVSLEPWGDEERLR
VQFLAQQSLSLAPVTAATARTALSGLSGADGRREERGRGKSWVCLSLG
GSGNTEPQKKGLSCRLWDSDILCLPGDIVPAPGPVLAPTHLQTELVLR
CQKETDCDLCLRVAVHLAVHGHWEEPEDEEKFGGAADSGVEEPRNASL
QAQVVLSFQAYPTARCVLLEVQVPAALVQFGQSVGSVVYDCFEAALGS
EVRIWSYTQPRYEKELNHTQQLPDCRGLEVWNSIPSCWALPWLNVSAD
GDNVHLVLNVSEEQHFGLSLYWNQVQGPPKPRWHKNLTGPQIITLNHT
DLVPCLCIQVWPLEPDSVRTNICPFREDPRAHQNLWQAARLQLLTLQS
WLLDAPCSLPAEAALCWRAPGGDPCQPLVPPLSWENVTVDKVLEFPLL
KGHPNLCVQVNSSEKLQLQECLWADSLGPLKDDVLLLETRGPQDNRSL
CALEPSGCTSLPSKASTRAARLGEYLLQDLQSGQCLQLWDDDLGALWA
CPMDKYIHKRWALVWLACLLFAAALSLILLLKKDHAKGWLRLLKQDVR
SGAAARGRAALLLYSADDSGFERLVGALASALCQLPLRVAVDLWSRRE
LSAQGPVAWFHAQRRQTLQEGGVVVLLFSPGAVALCSEWLQDGVSGPG
AHGPHDAFRASLSCVLPDFLQGRAPGSYVGACFDRLLHPDAVPALFRT
VPVFTLPSQLPDFLGALQQPRAPRSGRLQERAEQVSRALQPALDSYFH
PPGTPAPGRGVGPGAGPGAGDGT.

Also provided is an IL-17RC polypeptide in which the signal sequence is removed (e.g., processed) or in which amino acids 1-20 are deleted, and optionally other deletions, insertions and substitutions. Another exemplary IL-17RC polypeptide includes the extracellular domain of IL-17RC, e.g., about amino acids 21-538 of SEQ ID NO:14. Other exemplary IL-17RC sequences include murine (Q8K4C2), rat (XP_{—}216240.5, GenBank) and bovine sequences (NP_{—}001068646.1, GenBank).

IL-17RD. An exemplary human IL-17RD receptor sequence is described by Uniprot identifier Q8NFM7. See also Xiong et al., J. Biol. Chem. 278:50273-50282 (2003). Also provided is an IL-17RD polypeptide in which the signal sequence is removed (e.g., processed) or in which amino acids 1-16 are deleted, and optionally other deletions, insertions and substitutions. Another exemplary IL-17RD polypeptide includes the extracellular domain of IL-17RD, e.g., about amino acids 17-299 of Q8NFM7.

IL-17RE. An exemplary human IL-17RE receptor sequence is described by UniProt identifier Q8NFR9. Also provided is an IL-17RE polypeptide in which the signal sequence is removed (e.g., processed) or in which amino acids 1-23 are deleted, and optionally other deletions, insertions and substitutions. Another exemplary IL-17RE polypeptide includes the extracellular domain of IL-17RE, e.g., about amino acids 24-454 of Q8NFR9.

The present invention provides novel antagonists of IL-17 receptor signaling, e.g., antagonists of one or more of IL-17RA, IL-17RB, IL-17RC, IL-17RD, and IL-17RE signaling, and the use of such antagonists in the treatment of inflammatory diseases and autoimmune diseases. The present invention further provides novel antagonists of IL-17 cytokine signaling, e.g., IL-17A, IL-17B, IL-17C, IL-17D, IL-17E, and IL-17F signaling, and their uses in the treatment of inflammatory disease and autoimmune disease.

The antagonists of the present invention, an exemplary member of which is an antagonist to IL-17RA, and including the neutralizing anti-IL-17RA designer cytokine antagonists of the present invention, can be used to block, inhibit, reduce, antagonize or neutralize the activity of IL-17A, IL-17F, or IL-17A/F or any combination therein in the treatment of inflammation and inflammatory diseases such as multiple sclerosis, cancer (particularly as characterized by the expression of IL-17 and/or IL-23), psoriasis, psoriatic arthritis, rheumatoid arthritis, autoimmune ocular diseases, endotoxemia, IBS, and inflammatory bowel disease (IBD), colitis, asthma, COPD, cystic fibrosis, allograft rejection, immune mediated renal diseases, hepatobiliary diseases, atherosclerosis, promotion of tumor growth, or degenerative joint disease, atherosclerosis, and other inflammatory conditions disclosed herein.

The present invention provides isolated polypeptides that bind to the contact surfaces of IL-17 ligands and/or receptors, thereby preventing their productive interaction. More specifically, the present invention provides polypeptides that bind to IL-17 ligands and/or receptors and inhibit the production of an inflammatory mediator in a cell expressing IL-17 receptors.

The five IL-17 receptors (IL-17RA-IL-17RE) are not homologous to any known receptors, and exhibit considerable sequence divergence. All appear to contain extracellular domains composed of fibronectin type-III (FnIII) domains, and cytoplasmic SEF/IL-17R (SEFIR) domains that show loose homology to Toll/IL-1R (TLR) domains (13,14). The IL-17 receptors mediate signaling events that are distinct from those triggered by the more widely known receptors for type I four helix cytokines (15, 16). Like TLR stimulation, IL-17 receptor stimulation results in activation of NF-κB and mitogen-activated protein kinases (MAPK). However, IL-17 receptor signaling does not utilize the same set of membrane proximal adaptor molecules as TLR signaling; IL-17R requires the adaptor Act1 which also contains a SEFIR domain (17-19). These unique signaling properties of IL-17 receptors enable T_{H}-17 cells to act as a bridge between innate and adaptive immune cells.

Mechanistically, fluorescence resonance energy transfer (FRET) studies have suggested that IL-17RA may exist as a preformed dimer on the cell surface that undergoes a conformational change upon IL-17 binding to form a heterodimeric signaling complex with IL-17RC. However, the molecular basis for how a homodimeric IL-17 cytokine would pair with two different receptors remains unknown (14, 20). The structural and biochemical analysis provided herein enables for the first time the rational design of specific antagonists of the IL-17 system. On the basis of this analysis we provide a suite of antagonists that are useful in interrupting IL-17 signaling and in treating mammals with a variety of diseases.

Preferred embodiments of the invention include binding peptides, proteins, and any fragments or permutations thereof that bind to an IL-17R or an IL-17 cytokine referred to interchangeably as “IL-17R antagonists”, “IL-17 antagonists”, “IL-17R neutralizing entities”, “IL-17R designer cytokine antagonists”, and “IL-17 designer cytokine antagonists.” Specifically, in some embodiments, such binding peptides or proteins are capable of specifically binding to a human IL-17R and are referred to as “IL-17R binding proteins.” Further, these binding peptides or proteins are capable of modulating biological activities associated with IL-17, e.g., antagonizing IL-17 activation of an IL-17 receptor, and thus are useful in the treatment of various diseases and pathological conditions such as inflammation and immune-related diseases. Exemplary antagonists have an IC50 of less than 200, 50, 20, or 10 nM.

In still another embodiment, the invention concerns an isolated polynucleotide that encodes a polypeptide of the present invention, wherein said polypeptide is capable of binding to IL-17R, e.g., IL-17RA, IL-17RB, IL-17RC, IL-17RD, or IL-17RE, and reducing its signaling capability.

The present invention also provides fusion proteins, comprising an antagonist of the present invention and an immunoglobulin moiety, e.g., an immunoglobulin domain or region. In such fusion proteins, the immunoglobulin moiety may be an immunoglobulin heavy chain constant region, such as a human F_{c} fragment. The present invention further includes isolated nucleic acid molecules that encode such fusion proteins.

The present invention also provides protein conjugates comprising an antagonist of the present invention conjugated to a polymer of polyethylene glycol.

The present invention further includes pharmaceutical compositions, comprising a pharmaceutically acceptable carrier and an IL-17R antagonist described herein.

In another aspect, the invention concerns a method for the treatment of an inflammatory disease characterized by elevated expression of IL-17 and/or IL-23 and/or IFN-γ in a mammalian subject, comprising administering to the subject an effective amount of an antagonist of IL-17 signaling.

In yet another embodiment, the invention concerns a method for inhibiting the production of an inflammatory mediator in a mammalian cell by treating the cell or its media with an antagonist of IL-17R.

In another aspect, the invention concerns a method for the treatment of an inflammatory disease characterized by elevated expression of IL-17 and/or IL-23 and/or IFN-γ in a mammalian subject, comprising administering to the subject an effective amount of an antagonist of IL-17 signaling.

Typical methods of the invention include methods to treat pathological conditions or diseases in mammals associated with or resulting from increased or enhanced IL-17 and/or IL-23 and/or IFN-γ expression and/or activity. In the methods of treatment, the antagonists of the present invention may be administered which preferably reduce the respective receptor activation. The methods contemplate the use of an antagonist of IL-17R that reduces signaling by blocking IL-17R complex formation.

Antagonists of the present invention (e.g., antagonists of IL-17R) are also useful to prepare medicines and medicaments for the treatment of immune-related and inflammatory diseases, including for example, systemic lupus erythematosis, arthritis, rheumatoid arthritis, osteoarthritis, psoriasis, demyelinating diseases of the central and peripheral nervous systems such as multiple sclerosis, idiopathic demyelinating polyneuropathy or Guillain-Barre syndrome, inflammatory bowel disease, colitis, ulcerative colitis, Crohn's disease, gluten-sensitive enteropathy, autoimmune ocular diseases, cancer, neoplastic diseases, atherosclerosis, and angiogenesis.

In a specific aspect, such medicines and medicaments comprise a therapeutically effective amount of an IL-17R antagonist with a pharmaceutically acceptable carrier. Preferably, the admixture is sterile.

In yet another embodiment, the invention concerns a method for inhibiting IL-17 production and/or maintenance by treating the T cells with an IL-17R antagonist.

In a still further embodiment, the invention provides a method of decreasing the activity of T-lymphocytes in a mammal comprising administering to said mammal an IL-17R antagonist, such as an IL-17R binding protein that comprises a sequence homologous to an IL-17 cytokine sequence, wherein the activity of T-lymphocytes in the mammal is decreased.

In a still further embodiment, the invention provides a method of decreasing the proliferation of T-lymphocytes in a mammal comprising administering to said mammal an IL-17R antagonist, such as an IL-17R binding protein that comprises a sequence homologous to an IL-17 cytokine sequence, wherein the proliferation of T-lymphocytes in the mammal is decreased.

Processes for producing the same are also herein described, wherein those processes comprise culturing a host cell comprising a vector which contains the appropriate encoding nucleic acid molecule under conditions suitable for expression of said antibody and recovering said antibody from the cell culture.

### IL-17R Binding Proteins

An IL-17 cytokine can include at least three sites that contact an IL-17R on one of its receptor binding faces. The IL-17 generally includes two subunits (here designated Chain A and B), each contributing amino acids to a particular receptor binding face. The use of the terms “Chain A” and “Chain B” is merely for reference. For example, in embodiments using a single chain format, “Chain A” may be placed C-terminal to “Chain B” and alternatively it may be place N-terminal to “Chain B.”

The IL-17 interface that binds IL-17RA includes three sites (Site 1, Site 2, and Site3) which include the following contact residues as shown in Table 1 (according to the numbering of IL-17F and SEQ ID NO:12):

TABLE 1

| | Chain A | Chain B |
|---|---|---|
| Site 1 | MET25, LYS115 | ILE29, ILE31, TRP58, ASN61, |
| | | TYR63, PRO64, SER65, GLU66, |
| | | VAL100, ARG102, HIS104, |
| | | VAL109, PHE111 |
| Site 2 | GLN94, GLN95, GLU96, | GLN36, ARG37, MET40, SER41, |
| | LYS115, LEU117 | ASN43, GLU45, TYR54, VAL56, |
| | | GLU66, VAL68, VAL118 |
| Site 3 | LEU75, ILE86, SER87, ASN89, | MET40, ARG42, ILE44, ARG47 |
| | VAL91, VAL125, PRO127, | |
| | VAL128, ILE129, HIS130, | |
| | HIS131, VAL132 | |

Certain residues are at the junction of two adjacent sites and accordingly are listed for both the sites. Several of the interface residues are buried upon binding to IL-17RA as shown in the table in Example 20 below.

In one aspect, this disclosure features an IL-17R binding protein that comprises an IL-17 cytokine including two subunits wherein one receptor binding face of the dimer formed by the two subunits includes one or more substitutions, e.g., at least two or three substitutions, e.g., non-conservative substitutions or a substitutions described herein. For example, the cytokine has at least one, two, three, four, five, six, or seven substitutions (or deletions) at the positions identified in Table 1 above, e.g. between two to ten, two-seven, or three to ten, or three to six. In some cases, one cytokine subunit differs from the other subunit at at least one, two, three, four, five, six, or seven substitutions (or deletions). For example, in the IL-17R binding protein, the two receptor binding faces can include different amino acids, e.g., at least one, two, three, four, five, six, or seven differences, e.g., at positions corresponding to those in Table 1.

One or both the subunits can have one or more conservative and/or one or more non-conservative substitutions. Typically, at least one subunit or both subunits are at least 90, 92, 94, 95, 96, 97, or 98% identical, but not 100% identical to a mature human IL-17, e.g., SEQ ID NO:2, 4, 6, 8, 10, 12, or 20. In one embodiment, neither subunits is 100% identical to a mature human IL-17, e.g., they differ by at least one, two, or three amino acids from the human IL-17 from which they were derived. In one embodiment, one subunit differs a mature human IL-17, whereas the other subunit is identical to a mature human IL-17. In certain embodiments, the substitutions in a subunit are not to residues in a corresponding murine protein.

### Site 1

In one embodiment, an IL-17R binding protein comprises an IL-17 cytokine including two subunits in which Site 1 of one receptor binding face includes one or more mutations, e.g., at least two or three mutations, e.g., non-conservative mutations or a mutation described herein. For example, one or more of the following Site 1 residues (identified based on the numbering for IL-17F and SEQ ID NO:12) are mutated: Chain A: MET25 and LYS115; and Chain B: ILE29, ILE31, TRP58, ASN61, TYR63, PRO64, SER65, GLU66, VAL100, ARG102, HIS104, VAL109, and PHE111, and corresponding residues in IL-17A, IL-17B, IL-17C, IL-17D, and IL-17E as shown in FIG. 4D. In one embodiment, the binding protein includes at least one mutation in one of the foregoing Chain A residues of Site 1 and at least one mutation in one of the foregoing Chain B residues. Some exemplary mutations that can be made in Site 1 include:

MET25 in Chain A can be mutated to another amino acid, e.g., alanine or an amino acid other than alanine. For example, MET25 is mutated to a neutral hydrophilic residue, a small aliphatic residue, a charged residue, or an aromatic residue. For example, MET25 is mutated to Trp or Tyr. MET25 can be mutated to disrupt hydrophobic packing near the surface, e.g., by mutation to a charged residue or to a bulky aromatic. Corresponding or non-conservative mutations can be made to VAL23 of SEQ ID NO:2, ARG36 of SEQ ID NO:4, LEU48 of SEQ ID NO:6, LEU36 of SEQ ID NO:8, and LEU33 of SEQ ID NO:10.

ILE29 in Chain B can be mutated to another amino acid, e.g., alanine or an amino acid other than alanine. For example, ILE29 is mutated to a neutral hydrophilic residue, a small aliphatic residue, a charged residue, or an aromatic residue. Corresponding or non-conservative mutations can be made to ILE27 of SEQ ID NO:2.

ILE31 in Chain B can be mutated to another amino acid, e.g., alanine or an amino acid other than alanine. For example, ILE31 is mutated to a small aliphatic residue, a charged residue, or an aromatic residue. Corresponding or non-conservative mutations can be made to ASN29 of SEQ ID NO:2.

TRP58 in Chain B can be mutated to another amino acid, e.g., alanine or an amino acid other than alanine. For example, TRP58 is mutated to a small aliphatic residue. Corresponding or non-conservative mutations can be made to GLU56 of SEQ ID NO:2, HIS85 of SEQ ID NO:4, THR97 of SEQ ID NO:6, TYR85 of SEQ ID NO:8, and ARG67 of SEQ ID NO:10.

ASN61 in Chain B can be mutated to another amino acid, e.g., alanine or an amino acid other than alanine. For example, ASN61 is mutated to an aliphatic residue, a charged residue, or an aromatic residue. Corresponding or non-conservative mutations can be made to GLU59 of SEQ ID NO:2, SER88 of SEQ ID NO:4, ASP100 of SEQ ID NO:6, ALA88 of SEQ ID NO:8, and ASN70 of SEQ ID NO:10.

TYR63 in Chain B can be mutated to another amino acid, e.g., alanine or an amino acid other than alanine. For example, TYR63 is mutated to an aliphatic residue, a neutral hydrophilic residue, or a charged residue. For example, TYR63 is mutated to Ala or Lys. Corresponding or non-conservative mutations can be made to TYR61 of SEQ ID NO:2, ILE90 of SEQ ID NO:4, TYR102 of SEQ ID NO:6, TYR90 of SEQ ID NO:8, and LEU72 of SEQ ID NO:10.

PRO64 in Chain B can be mutated to another amino acid, e.g., alanine or an amino acid other than alanine. For example, PRO64 is mutated to glycine, an aliphatic residue, a neutral hydrophilic residue, a charged residue, or an aromatic residue. Corresponding or non-conservative mutations can be made to PRO62 of SEQ ID NO:2, PRO91 of SEQ ID NO:4, PRO103 of SEQ ID NO:6, PRO91 of SEQ ID NO:8, and PRO73 of SEQ ID NO:10.

SER65 in Chain B can be mutated to another amino acid, e.g., alanine or an amino acid other than alanine. For example, SER65 is mutated to an aliphatic residue, particularly a large aliphatic residue, a charged residue, or an aromatic residue. For example, SER65 is mutated to Lys or Trp. Corresponding or non-conservative mutations can be made to SER63 of SEQ ID NO:2, VAL92 of SEQ ID NO:4, GLN104 of SEQ ID NO:6, ARG92 of SEQ ID NO:8, and GLN74 of SEQ ID NO:10.

VAL100 in Chain B can be mutated to another amino acid, e.g., alanine or an amino acid other than alanine. For example, VAL100 is mutated to a neutral hydrophilic residue, a large aliphatic residue, a charged residue, or an aromatic residue. Corresponding or non-conservative mutations can be made to LEU98 of SEQ ID NO:2, ARG128 of SEQ ID NO:4, LEU140 of SEQ ID NO:6, LEU128 of SEQ ID NO:8, and PHE111 of SEQ ID NO:10.

ARG102 in Chain B can be mutated to another amino acid, e.g., alanine or an amino acid other than alanine. For example, ARG102 is mutated to an aliphatic residue, a neutral hydrophilic residue, an acidic residue, or an aromatic residue. For example, ARG102 is mutated to Ala, Ser, Gln, or Asn. Corresponding mutations can be made to ARG100 of SEQ ID NO:2, ARG130 of SEQ ID NO:4, ARG142 of SEQ ID NO:6, ARG130 of SEQ ID NO:8, and ARG113 of SEQ ID NO:10.

HIS104 in Chain B can be mutated to another amino acid, e.g., alanine or an amino acid other than alanine. For example, HIS104 is mutated to an aliphatic residue or an acidic residue. For example, HIS104 is mutated to Glu or Asp. Corresponding or non-conservative mutations can be made to PRO102 of SEQ ID NO:2, PRO136 of SEQ ID NO:4, PRO153 of SEQ ID NO:6, CYS134 of SEQ ID NO:8, and GLY121 of SEQ ID NO:10.

VAL109 in Chain B can be mutated to another amino acid, e.g., alanine or an amino acid other than alanine. For example, VAL109 is mutated to a neutral hydrophilic residue, a large aliphatic residue, a charged residue, or an aromatic residue.

PHE111 in Chain B can be mutated to another amino acid, e.g., alanine or an amino acid other than alanine. For example, PHE111 is mutated to a small aliphatic residue, a neutral hydrophilic residue, or a charged residue. For example, PHE111 is mutated to Ala. Corresponding or non-conservative mutations can be made to PHE109 of SEQ ID NO:2, GLN143 of SEQ ID NO:4, PHE160 of SEQ ID NO:6, TYR141 of SEQ ID NO:8, and LEU128 of SEQ ID NO:10.

GLU66 in Chain B can be mutated to another amino acid, e.g., alanine or an amino acid other than alanine. For example, GLU66 is mutated to an aliphatic residue, a neutral hydrophilic residue, a basic residue, or an aromatic residue. For example, a mutation is made to disrupt hydrogen bonding by GLU66. Corresponding or non-conservative mutations can be made to VAL64 of SEQ ID NO:2, ASP93 of SEQ ID NO:4, LYS105 of SEQ ID NO:6, TYR93 of SEQ ID NO:8, and ASP75 of SEQ ID NO:10.

LYS115 in Chain A can be mutated to another amino acid, e.g., alanine or an amino acid other than alanine. For example, LYS115 is mutated to an aliphatic residue, a neutral hydrophilic residue, an acidic residue, or an aromatic residue. For example, LYS115 is mutated to Ala. Corresponding or non-conservative mutations can be made to LYS113 of SEQ ID NO:2, MET147 of SEQ ID NO:4, PHE164 of SEQ ID NO:6, TYR145 of SEQ ID NO:8, and LEU132 of SEQ ID NO:10.

### Site 2

In one embodiment, an IL-17R binding protein comprises an IL-17 cytokine including two subunits in which Site 2 of one receptor binding face includes one or more mutations, e.g., at least two or three mutations, e.g., non-conservative mutations or a mutation described herein. For example, one or more of the following Site 2 residues (identified based on the numbering for IL-17F and SEQ ID NO:12) are mutated: Chain A: GLN94, GLN95, GLU96, LYS115, and LEU117; and Chain B: GLN36, ARG37, MET40, SER41, ASN43, GLU45, TYR54, VAL56, GLU66, VAL68, and VAL118, and corresponding residues in IL-17A, IL-17B, IL-17C, IL-17D, and IL-17E as shown in FIG. 4D. In one embodiment, the binding protein includes at least one mutation in one of the foregoing Chain A residues of Site 2 and at least one mutation in one of the foregoing Chain B residues. Some exemplary mutations that can be made in Site 2 include:

GLN36 in Chain B can be mutated to another amino acid, e.g., alanine or an amino acid other than alanine. For example, GLN36 is mutated to an aliphatic residue, a charged residue, or an aromatic residue. For example, GLN36 is mutated to disrupt hydrogen bonding by this residue. Corresponding or non-conservative mutations can be made to THR34 of SEQ ID NO:2, MET47 of SEQ ID NO:4, GLY59 of SEQ ID NO:6, PRO47 of SEQ ID NO:8, and SER44 of SEQ ID NO:10.

ARG37 in Chain B can be mutated to another amino acid, e.g., alanine or an amino acid other than alanine. For example, ARG37 is mutated to an aliphatic residue, a neutral hydrophilic residue, an acidic residue, or an aromatic residue. For example, ARG37 is mutated to Ala or Glu. Corresponding or non-conservative mutations can be made to ASN35 of SEQ ID NO:2, VAL48 of SEQ ID NO:4, ARG60 of SEQ ID NO:6, ARG48 of SEQ ID NO:8, and CYS45 of SEQ ID NO:10.

MET40 in Chain B can be mutated to another amino acid, e.g., alanine or an amino acid other than alanine. For example, MET40 is mutated to a neutral hydrophilic residue, a small aliphatic residue, a charged residue, or an aromatic residue. Corresponding or non-conservative mutations can be made to ARG38 of SEQ ID NO:2, LEU51 of SEQ ID NO:4, ARG63 of SEQ ID NO:6, ALA51 of SEQ ID NO:8, and SER48 of SEQ ID NO:10. For example, ARG38 of SEQ ID NO:2 and ARG63 of SEQ ID NO:6 can be mutated to Glu, Asp, Gln, Asn, Thr, or Ser or to another residue that disrupts its ability to hydrogen bond or form salt bridges.

SER41 in Chain B can be mutated to another amino acid, e.g., alanine or an amino acid other than alanine. For example, SER41 is mutated to an aliphatic residue, a charged residue, or an aromatic residue. For example, SER41 is mutated to Ala, Trp, Tyr, Arg or Lys. Corresponding or non-conservative mutations can be made to SER39 of SEQ ID NO:2.

ASN43 in Chain B can be mutated to another amino acid, e.g., alanine or an amino acid other than alanine. For example, ASN43 is mutated to an aliphatic residue, a charged residue, or an aromatic residue. For example, ASN43 is mutated to Glu or Asp. Corresponding or non-conservative mutations can be made to ASP41 of SEQ ID NO:2, MET70 of SEQ ID NO:4, ASP82 of SEQ ID NO:6, PRO70 of SEQ ID NO:8, and PRO52 of SEQ ID NO:10. For example, ASP41 of SEQ ID NO:2 can be mutated to Ile, Leu, Tyr, Arg or Lys.

GLU45 in Chain B can be mutated to another amino acid, e.g., alanine or an amino acid other than alanine. For example, GLU45 is mutated to an aliphatic residue or an aromatic residue. Corresponding or non-conservative mutations can be made to TYR43 of SEQ ID NO:2, ASN72 of SEQ ID NO:4, HIS84 of SEQ ID NO:6, ASN72 of SEQ ID NO:8, and ASN54 of SEQ ID NO:10.

TYR54 in Chain B can be mutated to another amino acid, e.g., alanine or an amino acid other than alanine. For example, TYR54 is mutated to an aliphatic residue, a neutral hydrophilic residue, or a charged residue. Corresponding or non-conservative mutations can be made to LEU52 of SEQ ID NO:2, TYR81 of SEQ ID NO:4, TYR93 of SEQ ID NO:6, TYR81 of SEQ ID NO:8, and TYR63 of SEQ ID NO:10.

VAL56 in Chain B can be mutated to another amino acid, e.g., alanine or an amino acid other than alanine. For example, VAL56 is mutated to a neutral hydrophilic residue, a large aliphatic residue, a charged residue, or an aromatic residue. Corresponding or non-conservative mutations can be made to ARG54 of SEQ ID NO:2, ILE83 of SEQ ID NO:4, VAL95 of SEQ ID NO:6, ILE83 of SEQ ID NO:8, and LEU65 of SEQ ID NO:10.

VAL68 in Chain B can be mutated to another amino acid, e.g., alanine or an amino acid other than alanine. For example, VAL68 is mutated to a neutral hydrophilic residue, a large aliphatic residue, a charged residue, or an aromatic residue. For example, VAL68 is mutated to Gln, Asn, Ser, or Thr. Corresponding or non-conservative mutations can be made to TRP66 of SEQ ID NO:2, PRO95 of SEQ ID NO:4, ALA107 of SEQ ID NO:6, PRO95 of SEQ ID NO:8, and TYR77 of SEQ ID NO:10.

GLN94 in Chain A can be mutated to another amino acid, e.g., alanine or an amino acid other than alanine. For example, GLN94 is mutated to an aliphatic residue, a charged residue, or an aromatic residue. Corresponding or non-conservative mutations can be made to GLN92 of SEQ ID NO:2, PHE122 of SEQ ID NO:4, LEU134 of SEQ ID NO:6, TYR122 of SEQ ID NO:8, and TYR105 of SEQ ID NO:10.

GLN95 in Chain A can be mutated to another amino acid, e.g., alanine or an amino acid other than alanine. For example, GLN95 is mutated to an aliphatic residue, a charged residue, or an aromatic residue. For example, GLN95 is mutated to Asp, Glu, Ala or Trp. Corresponding or non-conservative mutations can be made to GLN93 of SEQ ID NO:2, SER123 of SEQ ID NO:4, GLN135 of SEQ ID NO:6, MET123 of SEQ ID NO:8, and HIS106 of SEQ ID NO:10.

GLU96 in Chain A can be mutated to another amino acid, e.g., alanine or an amino acid other than alanine. For example, GLU96 is mutated to an aliphatic residue, a basic residue, or an aromatic residue. Corresponding or non-conservative mutations can be made to GLU94 of SEQ ID NO:2, GLN124 of SEQ ID NO:4, SER136 of SEQ ID NO:6, PRO124 of SEQ ID NO:8, and ASN107 of SEQ ID NO:10.

LEU117 in Chain A can be mutated to another amino acid, e.g., alanine or an amino acid other than alanine. For example, LEU117 is mutated to a neutral hydrophilic residue, a small aliphatic residue, a charged residue, or an aromatic residue. Corresponding or non-conservative mutations can be made to LEU115 of SEQ ID NO:2, THR149 of SEQ ID NO:4, HIS166 of SEQ ID NO:6, THR147 of SEQ ID NO:8, and ARG134 of SEQ ID NO:10.

VAL118 in Chain B can be mutated to another amino acid, e.g., alanine or an amino acid other than alanine. For example, VAL118 is mutated to a neutral hydrophilic residue, a large aliphatic residue, a charged residue, or an aromatic residue. Corresponding or non-conservative mutations can be made to VAL116 of SEQ ID NO:2, ILE150 of SEQ ID NO:4, VAL167 of SEQ ID NO:6, ILE148 of SEQ ID NO:8, and VAL135 of SEQ ID NO:10.

In addition, for example, LYS37 of SEQ ID NO:2 can be mutated to another amino acid, e.g., alanine or an amino acid other than alanine. For example, it can be mutated to Glu, Asp, Gln, Asn, Thr, or Ser or to another residue that disrupts its ability to hydrogen bond or form salt bridges.

ARG30 of SEQ ID NO:2 and ARG40 of SEQ ID NO:10 can be mutated to another amino acid, e.g., alanine or an amino acid other than alanine. For example, it can be mutated to Glu, Asp, Gln, Asn, Thr, or Ser or to another residue that disrupts its ability to hydrogen bond or form salt bridges.

### Site 3

In one embodiment, an IL-17R binding protein comprises an IL-17 cytokine including two subunits in which Site 3 of one receptor binding face includes one or more mutations, e.g., at least two or three mutations, e.g., non-conservative mutations or a mutation described herein. For example, one or more of the following Site 3 residues (identified based on the numbering for IL-17F and SEQ ID NO:12) are mutated: Chain A: LEU75, ILE86, SER87, ASN89, VAL91, VAL125, PRO127, VAL128, ILE129, HIS130, HIS131, and VAL132, and/or Chain A can be truncated at a residue preceding VAL125, THR126, PRO127, VAL128, ILE129, HIS130, HIS131, or VAL132; and Chain B: MET40, ARG42, ILE44, and ARG47, and corresponding residues in IL-17A, IL-17B, IL-17C, IL-17D, and IL-17E as shown in FIG. 4D. In one embodiment, the binding protein includes at least one mutation in one of the foregoing Chain A residues of Site 3 and at least one mutation in one of the foregoing Chain B residues.

Some exemplary mutations that can be made in Site 3 include:

ARG42 in Chain B can be mutated to another amino acid, e.g., alanine or an amino acid other than alanine. For example, ARG42 is mutated to an aliphatic residue, a neutral hydrophilic residue, an acidic residue, or an aromatic residue. For example, ARG42 is mutated to Glu, Asp, Trp, or Ala. Corresponding or non-conservative mutations can be made to SER40 of SEQ ID NO:2, TRP69 of SEQ ID NO:4, ALA81 of SEQ ID NO:6, PRO69 of SEQ ID NO:8, and GLY51 of SEQ ID NO:10.

ILE44 in Chain B can be mutated to another amino acid, e.g., alanine or an amino acid other than alanine. For example, ILE44 is mutated to a neutral hydrophilic residue, a small aliphatic residue, a charged residue, or an aromatic residue. Corresponding or non-conservative mutations can be made to TYR42 of SEQ ID NO:2, SER71 of SEQ ID NO:4, THR83 of SEQ ID NO:6, THR71 of SEQ ID NO:8, and LEU53 of SEQ ID NO:10.

ARG47 in Chain B can be mutated to another amino acid, e.g., alanine or an amino acid other than alanine. For example, ARG47 is mutated to an aliphatic residue, a neutral hydrophilic residue, an acidic residue, or an aromatic residue. For example, ARG47 is mutated to Glu, Asp, Gln, or Asn. Corresponding or non-conservative mutations can be made to ARG45 of SEQ ID NO:2, ARG74 of SEQ ID NO:4, ARG86 of SEQ ID NO:6, ARG74 of SEQ ID NO:8, and ARG56 of SEQ ID NO:10.

LEU75 in Chain A can be mutated to another amino acid, e.g., alanine or an amino acid other than alanine. For example, LEU75 is mutated to a neutral hydrophilic residue, a small aliphatic residue, a charged residue, or an aromatic residue. Corresponding or non-conservative mutations can be made to LEU73 of SEQ ID NO:2, LEU102 of SEQ ID NO:4, ARG114 of SEQ ID NO:6, ARG102 of SEQ ID NO:8, and PRO84 of SEQ ID NO:10.

ILE86 in Chain A can be mutated to another amino acid, e.g., alanine or an amino acid other than alanine. For example, ILE86 is mutated to a neutral hydrophilic residue, a small aliphatic residue, or a charged residue. Corresponding or non-conservative mutations can be made to TYR84 of SEQ ID NO:2, ARG114 of SEQ ID NO:4, ALA126 of SEQ ID NO:6, VAL114 of SEQ ID NO:8, and PRO97 of SEQ ID NO:10.

SER87 in Chain A can be mutated to another amino acid, e.g., alanine or an amino acid other than alanine. For example, SER87 is mutated to an aliphatic residue, a charged residue, or an aromatic residue. Corresponding or non-conservative mutations can be made to HIS85 of SEQ ID NO:2, SER115 of SEQ ID NO:4, ALA127 of SEQ ID NO:6, ARG115 of SEQ ID NO:8, and ARG98 of SEQ ID NO:10.

ASN89 in Chain A can be mutated to another amino acid, e.g., alanine or an amino acid other than alanine. For example, ASN89 is mutated to an aliphatic residue, a charged residue, or an aromatic residue. For example, ASN89 is mutated to Ala. Corresponding or non-conservative mutations can be made to ASN87 of SEQ ID NO:2, VAL117 of SEQ ID NO:4, ASN129 of SEQ ID NO:6, ARG117 of SEQ ID NO:8, and ASN100 of SEQ ID NO:10.

VAL91 in Chain A can be mutated to another amino acid, e.g., alanine or an amino acid other than alanine. For example, VAL91 is mutated to a neutral hydrophilic residue, a large aliphatic residue, a charged residue, or an aromatic residue. For example, VAL91 is mutated to Asp or Glu. Corresponding or non-conservative mutations can be made to VAL89 of SEQ ID NO:2, VAL119 of SEQ ID NO:4, VAL131 of SEQ ID NO:6, ALA119 of SEQ ID NO:8, and GLU102 of SEQ ID NO:10.

VAL125 in Chain A can be mutated to another amino acid, e.g., alanine or an amino acid other than alanine. For example, VAL125 is mutated to a neutral hydrophilic residue, a large aliphatic residue, a charged residue, or an aromatic residue. Corresponding or non-conservative mutations can be made to VAL123 of SEQ ID NO:2, ILE157 of SEQ ID NO:4, VAL174 of SEQ ID NO:6, VAL155 of SEQ ID NO:8, and VAL142 of SEQ ID NO:10.

PRO127 in Chain A can be mutated to another amino acid, e.g., alanine or an amino acid other than alanine. For example, PRO127 is mutated to an aliphatic residue, a neutral hydrophilic residue, a charged residue, or an aromatic residue. In one embodiment, PRO127 is deleted. Corresponding or non-conservative mutations can be made to PRO125 of SEQ ID NO:2, PRO176 of SEQ ID NO:6, GLU157 of SEQ ID NO:8, and PRO144 of SEQ ID NO:10.

VAL128 in Chain A can be mutated to another amino acid, e.g., alanine or an amino acid other than alanine. For example, VAL128 is mutated to a neutral hydrophilic residue, a large aliphatic residue, a charged residue, or an aromatic residue. In one embodiment, VAL128 is deleted. Corresponding or non-conservative mutations can be made to ILE126 of SEQ ID NO:2, ARG177 of SEQ ID NO:6, PRO158 of SEQ ID NO:8, and ARG145 of SEQ ID NO:10.

ILE129 in Chain A can be mutated to another amino acid, e.g., alanine or an amino acid other than alanine. For example, ILE129 is mutated to a neutral hydrophilic residue, a small aliphatic residue, a charged residue, or an aromatic residue. In one embodiment, ILE129 is deleted. Corresponding or non-conservative mutations can be made to VAL127 of SEQ ID NO:2, SER178 of SEQ ID NO:6, GLU159 of SEQ ID NO:8, and VAL146 of SEQ ID NO:10.

HIS130 in Chain A can be mutated to another amino acid, e.g., alanine or an amino acid other than alanine. For example, HIS130 is mutated to an aliphatic residue or an acidic residue. In one embodiment, HIS130 is deleted. Corresponding or non-conservative mutations can be made to HIS128 of SEQ ID NO:2, VAL179 of SEQ ID NO:6, LYS160 of SEQ ID NO:8, and MET147 of SEQ ID NO:10.

HIS131 in Chain A can be mutated to another amino acid, e.g., alanine or an amino acid other than alanine. For example, HIS131 is mutated to an aliphatic residue or an acidic residue. In one embodiment, HIS131 is deleted. Corresponding or non-conservative mutations can be made to HIS129 of SEQ ID NO:2, ASP161 of SEQ ID NO:8, and GLY148 of SEQ ID NO:10.

VAL132 in Chain A can be mutated to another amino acid, e.g., alanine or an amino acid other than alanine. For example, VAL132 is mutated to a neutral hydrophilic residue, a large aliphatic residue, a charged residue, or an aromatic residue. In one embodiment, VAL132 is deleted. Corresponding or non-conservative mutations can be made to VAL130 of SEQ ID NO:2, and ALA162 of SEQ ID NO:8.

The cytokine subunit can contain one or more deletions, e.g., at least two, three, four, or five between the following residues and the natural C-terminus of the subunit: PRO127 in SEQ ID NO:12, PRO125 of SEQ ID NO:2, PRO176 of SEQ ID NO:6, GLU157 of SEQ ID NO:8, and PRO144 of SEQ ID NO:10. In some embodiments, the cytokine subunit is truncated immediately after one of the forgoing positions or one, two, or three residues away from such position. The polypeptide containing the cytokine subunit can terminate at such truncation, or alternatively can include other exogenous sequences (such as a polypeptide tag) fused to the terminus of the truncated cytokine subunit.

Exemplary IL-17R binding proteins include a plurality of mutations, for example:
at least one, two, or three substitutions in Site 1 and at least one, two or three substitutions in Site 2;
at least one, two, or three substitutions in Site 1 and at least one, two or three substitutions or deletions in Site 3;
at least one, two, or three substitutions in Site 2 and at least one, two or three substitutions or deletions in Site 3;
at least one, two, or three substitutions in Site 1, at least one, two, or three mutations in Site 2, and at least one, two or three substitutions or deletions in Site 3.

Exemplary IL-17R binding proteins include a plurality of substitutions and/or deletions in an IL-17 cytokine. For example, an IL-17 binding protein can include at least two, three or four of the following features (according to the numbering in SEQ ID NO:12): (i) substitutions in Chain A at R47, (ii) substitutions in Chain A at S65, (iii) substitutions in Chain A at W68, (iv) substitutions in Chain A at R102, (v) substitutions in Chain B at N89, and (vi) deletion of at least two C-terminal residues of SEQ ID NO:12 or at least two, three, four, or five residues corresponding to 127-132 of SEQ ID NO:12. The protein can have still other features described herein.

Some exemplary mutated IL-17 cytokine sequences are listed in Examples 24-27. Sequences that are at least 85, 90, 92, 94, 96, 98, or 99% identical to such sequences and that include substitutions at the same positions as such sequences may also be used.

Corresponding mutations can be made in other IL-17 cytokines as indicated by the correspondence shown in FIG. 4D. In addition, the following residues are likely buried in the core of the IL-17 cytokine and in certain embodiments, at least 50, 60, 70, 80, 90, or 100% of these residues are not mutated:

| Cytokine | SEQ ID | core positions |
|---|---|---|
| IL-17F | 12 | SER48, THR49, SER50, PRO51, TRP52, ARG62, |
| | | ALA70, GLY76, CYS77, SER90, ILE93, THR97, |
| | | LEU98, VAL99, LEU113, THR119, VAL120, |
| | | GLY121, CYS122, THR123, and CYS124 |
| IL-17A | 2 | SER46, THR47, SER48, PRO49, TRP50, ARG60, |
| | | ALA68, GLY74, CYS75, SER88, ILE91, ILE95, |
| | | LEU96, VAL97, LEU111, SER117, VAL118, |
| | | GLY119, CYS120, THR121, and CYS122 |
| IL-17B | 4 | SER75, LEU76, SER77, PRO78, TRP79, ARG89, |
| | | ALA97, GLY103, CYS104, SER118, VAL121, |
| | | VAL125, PRO126, VAL127, ALA145, ALA151, |
| | | VAL152, GLY153, CYS154, THR155, and CYS156 |
| IL-17C | 6 | SER87, ILE88, SER89, PRO90, TRP91, ARG101, |
| | | ALA109, GLY115, CYS116, SER130, LEU133, |
| | | LEU137, LEU138, VAL139, THR162, PRO168, |
| | | VAL169, GLY170, CYS171, THR172, and CYS173 |
| IL-17D | 8 | SER75, VAL76, SER77, PRO78, TRP79, ARG89, |
| | | ALA97, GLY103, CYS104, SER118, VAL121, |
| | | THR125, VAL126, VAL127, GLU143, PRO149, |
| | | VAL150, GLY151, CYS152, THR153, and CYS154 |
| IL-17E | 10 | ALA57, ILE58, SER59, PRO60, TRP61, ARG71, |
| | | ALA79, HIS85, CYS86, SER101, LEU104, GLN108, |
| | | THR109, VAL110, ARG130, SER136, LEU137, |
| | | ALA138, CYS139, VAL140, and CYS141 |

In one embodiment, an IL-17R binding protein is used to detect an IL-17R, e.g., on the surface of a cell, in a sample, or in a patient. For example, the IL-17R binding protein can bind to and detect the IL-17R on the cell without agonizing the receptor. The IL-17R binding protein can be labeled.

In one embodiment, an IL-17R binding protein is used as a receptor antagonist, e.g., to bind to an IL-17 receptor subunit and prevent receptor dimerization.

### Amino Acid Modifications

Polypeptides described herein can be modified in a variety of ways including substitution, deletion, or addition. A substitution entails the replacement of one amino acid for another. Such replacements can be made using any one of the twenty amino acids directly encoded by the genetic code: alanine, arginine, asparagine, aspartic acid, cysteine, glutamic acid, glutamine, glycine, histidine, isoleucine, leucine, lysine, methionine, phenylalanine, proline, serine, threonine, tryptophan tyrosine, and valine. In addition, amino acids of a polypeptide can be replaced using amino acids not directly encoded by the genetic code for example: selenocysteine, pyrrolysine, p-nitrophenylalanine, p-sulfotyrosine, p-carboxyphenylalanine, o-nitrophenylalanine, 5-nitro His, 3-nitro Tyr, 2-nitro Tyr, nitro substituted Leu, nitro substituted His, nitro substituted Ile, nitro substituted Trp, 2-nitro Trp, 4-nitro Trp, 5-nitro Trp, 6-nitro Trp, 7-nitro Trp, aminotyrosines, and carboxyphenyalanines.

Conservative amino acid substitutions can frequently be made in a protein without altering either the conformation or the function of the protein. Substitutions can be chosen based on their potential effect on (a) backbone structure in the vicinity of the substitution, for example, a sheet or helical conformation, (b) the charge or hydrophobicity of the molecule at the target site, or (c) the volume and branching of the side chain.

Amino acid residues can be classified based on side-chain properties: (1) aliphatic: ala, met, val, leu, ile; (2) small aliphatic: ala, val; (3) large aliphatic: met, leu, ile; (4) neutral hydrophilic: ser, thr; asn; gln; (5) acidic: asp, glu; (6) basic: his, lys, arg; (7) charged: arg, asp, glu, his, lys; (8) residues that affect backbone conformation: gly, pro; and (9) aromatic: trp, tyr, phe. Non-conservative substitutions can include substituting a member of one of these classes for a member of a different class or making a substitution not identified in the table below. Conservative substitutions can include substituting a member of one of these classes for another member of the same class. Generally mutations are not made to Cys.

Exemplary conservative substitutions are described in the following table (with exemplary non-conservative substitutions being substitutions to residues not identified as conservative substitutions):

TABLE 2

| | | Further Specific |
|---|---|---|
| | Exemplary | and Examplary |
| Original | Substitutions | Substitutions |
| Ala (A) | val; leu; ile | val |
| Arg (R) | lys; gln; asn | lys |
| Asn (N) | gln; his; lys; arg | gln |
| Asp (D) | glu | glu |
| Cys (C) | ser, thr | ser |
| Gln (Q) | asn | asn |
| Glu (E) | asp | asp |
| Gly (G) | pro; ala | ala |
| His (H) | asn; gln; lys; arg | arg |
| Ile (I) | leu; val; met; ala; phe; leu | leu |
| Leu (L) | ile; val; met; ala; phe | ile |
| Lys (K) | arg; gln; asn | arg |
| Met (M) | leu; phe; ile | leu |
| Phe (F) | leu; val; ile; ala; tyr | leu |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe | ala |

### Heterodimer Formation

Any appropriate approach can be used to form heterodimers of two cytokine subunits described herein. Exemplary heterodimers include heterodimers of two different sequence variants of IL-17A, IL-17F, IL-17B, IL-17C, IL-17D, and IL-17E, as well as heterodimers that combine two different cytokine family members, e.g., a sequence variant of IL-17A and a wildtype or variant of IL-17F; a sequence variant of IL-17F and a wildtype or variant of IL-17A; and so forth.

One approach to forming heterodimers is to connect one of the two subunit to one sequence of a heterodimeric pair, and the other subunit to the other sequence of the pair. The exogenous heterodimerization sequence from the heterodimeric pair can be positioned N- or C-terminal to the cytokine subunit. For example, the heterodimeric pair is a non-cytokine protein, e.g., a heterodimerization domain of a transcription factor (e.g., fos/jun), a receptor, or an artificial sequence. An exemplary artificial sequence is an engineered acidic-basic zipper. Another exemplary heterodimerization approach is to use an Fc domain engineered to form a heterodimer, e.g., a knobs-in-hole modified CH3 domain, e.g., within an Fc domain or independently. See, e.g., Ridgway Protein Eng. 1996 July; 9(7):617-2. Still another approach includes attaching one cytokine subunit to the constant region of an immunoglobulin light chain, and the other cytokine subunit to the CH1 constant region of an immunoglobulin heavy chain.

Another approach to forming heterodimers is to connect the two subunits using a linker to form a single chain protein. The linker can be any appropriate length, e.g., at least 24, 25, 27, 29, 30 or 32 residues, e.g., between 25-34 or 27-37 residues. The linker can include a repeating sequence, e.g., (Gly-Gly-Ser)ₙ or (Gly-Gly-Gly Ser)ₙ or (Gly-Gly-Gly-Gly-Ser)ₙ where “n” is, e.g., 2, 3, 4, 5, 6, 7 or more. Longer and shorter linkers can also be used. Linker lengths with maximum stability and maximum heterodimer formation can be selected and used.

IL-17R binding proteins and other proteins described herein can be produced by expression in recombinant host cells, but also by other methods such as in vitro transcription and translation and chemical synthesis. For cellular expression, one or more nucleic acids (e.g., cDNA or genomic DNA) encoding a binding protein may be inserted into a replicable vector for cloning or for expression. Various vectors are publicly available. The vector may, for example, be a plasmid, cosmid, viral genome, phagemid, phage genome, or other autonomously replicating sequence. The appropriate coding nucleic acid sequence may be inserted into the vector by a variety of procedures. For example, appropriate restriction endonuclease sites can be engineered (e.g., using PCR). Then restriction digestion and ligation can be used to insert the coding nucleic acid sequence at an appropriate location. Vector components generally include one or more of an origin of replication, one or more marker genes, an enhancer element, a promoter, and a transcription termination sequence.

For bacterial expression, the binding protein can be produced with or without a signal sequence. For example, it can be produced within cells so that it accumulates in inclusion bodies. It can also be secreted, e.g., by addition of a prokaryotic signal sequence, e.g., an appropriate leader sequence such as from alkaline phosphatase, penicillinase, or heat-stable enterotoxin II. Exemplary bacterial host cells for expression include any transformable E. coli K-12 strain (such as E. coli C600, ATCC 23724; E. coli HB101 NRRLB-11371, ATCC-33694; E. coli MM294 ATCC-33625; E. coli W3110 ATCC-27325), strains of B. subtilis, Pseudomonas, and other bacilli. Proteins produced in bacterial systems will typically lack glycosylation.

The binding protein can be expressed in a yeast host cell, e.g., Saccharomyces cerevisiae, Schizosaccharomyces pombe, Hanseula, or Pichia pastoris. For yeast expression, the binding protein can also be produced intracellularly or by secretion, e.g., using the yeast invertase leader or alpha factor leader. In mammalian cell expression, mammalian signal sequences may be used to direct secretion of the protein, such as signal sequences from secreted polypeptides of the same or related species, as well as viral secretory leaders. Expression vectors used in eukaryotic host cells (yeast, fungi, insect, plant, animal, human, or nucleated cells from other multicellular organisms) can also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding the binding protein. The expression vector may also include one or more intronic sequences.

The binding protein can also be expressed in insect cells, e.g., Sf9 or SF21 cells, e.g., using the pFAST-BAC™ system. The binding protein can also be expressed in mammalian cells. For example, cell lines of mammalian origin also may be employed. Examples of mammalian host cell lines include the COS-7 line of monkey kidney cells (ATCC CRL 1651) (Gluzman et al., Cell 23:175, 1981), L cells, C127 cells, 3T3 cells (ATCC CCL 163), Chinese hamster ovary (CHO) cells, HeLa cells, and BHK (ATCC CRL 10) cell lines, and the CV1/EBNA cell line derived from the African green monkey kidney cell line CV1 (ATCC CCL 70) as described by McMahan et al. (EMBO J. 10: 2821, 1991). Established methods for introducing DNA into mammalian cells have been described (Kaufman, R. J., Large Scale Mammalian Cell Culture, 1990, pp. 1569).

Still other methods, vectors, and host cells suitable for adaptation to the synthesis of binding protein in recombinant cells are described in Molecular Cloning: A Laboratory Manual, Third Ed., Sambrook et al. (eds.), Cold Spring Harbor Press, (2001) (ISBN: 0879695773). IL-17 cytokine proteins can be expressed and purified by any appropriate method, e.g., in mammalian, fungal, or bacterial cells. The proteins can be glycosylated or not glycosylated.

Once expressed in cells, IL-17R binding proteins and proteins described herein can be recovered from culture medium, inclusion bodies, or cell lysates. Cells can be disrupted by various physical or chemical means, such as freeze-thaw cycling, sonication, mechanical disruption, or cell lysing agents (e.g., detergents). IL-17R binding proteins and proteins described herein can be purified from other cell proteins or polypeptides that can be found in cell lysates or in the cell medium. One exemplary purification procedure includes cation exchange chromatography and gel filtration. See, e.g., Murphy et al. Protein Expr Purif. 1998 March; 12(2):208-14. Various methods of protein purification may be employed and such methods are known in the art and described for example in Deutscher, Methods in Enzymology, 182 (1990); and Scopes, Protein Purification: Principles and Practice, Springer-Verlag, New York (2010) (ISBN: 1441928332). Purification moieties (such as epitope tags and affinity handles) can be optionally removed by proteolytic cleavage.

### Methods of Use

The compositions described herein are useful in methods for treating or preventing a disease or disorder in a vertebrate subject. In one such method, the step of administering to the subject a composition containing one or more polypeptides is provided. As described herein, the composition is administered intravesicularly, topically, orally, rectally, ocularly, optically, nasally, or via inhalation.

Also provided are methods of using the binding proteins described herein (such as IL-17R binding proteins, antibodies to an IL-17 cytokine member, and antibodies to an IL-17R) to modulate the immune system of a vertebrate. A level of an inflammatory cytokine can be reduced upon the administration of a modified polypeptide in a mammalian subject, such as by administering to the subject a therapeutically effective amount of a composition comprising a modified IL-17. Exemplary inflammatory cytokines are IL-1, IL-6, TNF-α, IL-17, IL-21, and IL-23. The level of inflammatory cytokine present in the blood and/or another tissue of the mammal is generally reduced. Modulation of the immune system also includes methods of increasing a level of an anti-inflammatory cytokine in a mammalian subject. For example, the anti-inflammatory cytokine is IL-10, IL-4, IL-11, IL-13, or TGF-β. Optionally, the level of the anti-inflammatory cytokine present in the blood of the mammal is increased.

In some aspects, an IL-17R binding protein or other engineered protein described herein is administered to a subject to treat a Th17 mediated disorder or a disorder mediated by an IL-17 cytokine family member. For example, the protein can be administered to a subject to treat atopic and contact dermatitis, colitis, endotoxemia, arthritis, rheumatoid arthritis, psoriatic arthritis, autoimmune ocular diseases (uveitis, scleritis), adult respiratory disease (ARD), demyelinating diseases, septic shock, multiple organ failure, inflammatory lung injury such as asthma, chronic obstructive pulmonary disease (COPD), airway hyper-responsiveness, chronic bronchitis, allergic asthma, psoriasis, eczema, IBS and inflammatory bowel disease (IBD) such as ulcerative colitis and Crohn's disease, diabetes, Helicobacter pylori infection, intra-abdominal adhesions and/or abscesses as results of peritoneal inflammation (i.e. from infection, injury, etc.), systemic lupus erythematosus (SLE), multiple sclerosis, systemic sclerosis, nephrotic syndrome, organ allograft rejection, graft vs. host disease (GVHD), kidney, lung, heart, etc. transplant rejection, streptococcal cell wall (SCW)-induced arthritis, osteoarthritis, gingivitis/periodontitis, herpetic stromal keratitis, restenosis, Kawasaki disease, and cancers/neoplastic diseases that are characterized by IL-17 and/or IL-23 expression, including but not limited to prostate, renal, colon, ovarian and cervical cancer, and leukemias (Tartour et al, Cancer Res. 5P:3698 (1999); Kato et al, Biochem. Biophys. Res. Commun. 282:735 (2001); Steiner et al, Prostate. 56:171 (2003); Langowksi et al, Nature 442: 461, 2006). For example, the binding protein is capable of binding, blocking, inhibiting, reducing, antagonizing or neutralizing IL-17 family members (either individually or together).

The compositions described herein may be used therapeutically or prophylactically. Cocktails of various different polypeptides can be used together to bind to and act upon one or multiple targets, e.g., multiple cell types, at once. Successful treatment can be assessed by routine procedures familiar to a physician.

In one embodiment, an IL-17R binding protein or other engineered protein (e.g., an antibody) described herein is administered to treat ocular disorders, including ocular disorders affecting the surface of the eye, ocular disorders mediated at least in part by an autoimmune reaction, ocular disorders associated with a systemic autoimmune disorder (such as Sjögren's syndrome and rheumatoid arthritis) or with a disorder associated with an IL-17 cytokine family member. The patient may or may not have other manifestations of a more systemic autoimmune disorder.

The ocular disorder can be a dry eye disorder that affects the surface of the eye. The disorder includes conditions also referred to keratoconjunctivitis sicca, keratitis sicca, sicca syndrome, xerophthalmia, tear film disorder, decreased tear production, aqueous tear deficiency, and Meibomian gland dysfunction. In addition, the binding proteins described herein can also be used to treat vernal conjunctivitis and inflammation associated with glaucoma.

Dry eye can include forms that are associated with Sjögren's syndrome (SS), e.g., Sjögren's syndrome associated keratoconjunctivitis sicca, but also forms that are not so associated, e.g., non-Sjögren's syndrome associated keratoconjunctivitis sicca. The patient may or may not have other manifestations of a systemic autoimmune disorder.

Subjects having a dry eye syndrome can exhibit inflammation of the eye dry, and can experience scratchy, stingy, itchy, burning or pressured sensations, irritation, pain, and redness. Dry eye can be associated with both excessive eye watering and conversely insufficient tear production. An IL-17R binding protein or other engineered protein (e.g., an antibody) described herein can be administered to such subjects to ameliorate or prevent the onset or worsening of one or more such symptoms.

An IL-17R binding protein or other engineered protein (e.g., an antibody) described herein can also be used to treat other disorders affecting the surface of the eye, such as the cornea. Such disorders include corneal ocular surface inflammatory conditions, corneal neovascularization, keratitis, including peripheral ulcerative keratitis and microbial keratitis. An IL-17R binding protein or other engineered protein (e.g., an antibody) described herein can be used to treat disorders affecting the conjunctiva, including conjunctival scarring disorders and conjunctivitis. The IL-17R binding protein or other engineered protein (e.g., an antibody) described herein can be used to treat still other disorders such as pemphigoid syndrome and Stevens-Johnson syndrome.

An IL-17R binding protein or other engineered protein (e.g., an antibody) described herein can be administered to a subject who is about to receive, undergoing, or recovering from a procedure involving the eye, e.g., corneal transplantation/keratoplasty, keratoprosthesis surgery, lamellar transplantation, selective endothelial transplantation. An IL-17R binding protein or other engineered protein (e.g., an antibody) described herein described herein can be administered to a subject to modulate neovascularization in or around the eye.

An IL-17R binding protein or other engineered protein (e.g., an antibody) described herein can be administered to a subject having an allergic reaction affecting the eye, e.g., a subject experiencing severe allergic (atopic) eye disease.

An IL-17R binding protein or other engineered protein (e.g., an antibody) described herein can be administered to a subject having an autoimmune disorder affecting the eye. Exemplary autoimmune ocular disorders include sympathetic ophthalmia, Vogt-Koyanagi Harada (VKH) syndrome, birdshot retinochoriodopathy, ocular cicatricial pemphigoid, Fuchs' heterochronic iridocyclitis, and various forms of uveitis. A IL-17R binding protein or other engineered protein (e.g., an antibody) described herein can be administered to a subject to treat any of the foregoing disorders.

Uveitis includes acute and chronic forms and includes inflammation of one or more of the iris, the ciliary body, and the choroid, and includes anterior, immediate, and posterior forms. Chronic forms may be associated with systemic autoimmune disease, e.g., Behcet's syndrome, ankylosing spondylitis, juvenile rheumatoid arthritis, Reiter's syndrome, and inflammatory bowel disease. A IL-17R binding protein or other engineered protein (e.g., an antibody) described herein can be administered to a subject to treat any of the foregoing forms of uveitis.

An IL-17R binding protein or other engineered protein (e.g., an antibody) described herein can be administered by any mode to treat an ocular disease. The agent can be delivered by a parenteral mode. Alternatively or in addition, the agent can be delivered directly to the eye or in the vicinity of the eye. For example, the protein can be administered topically or intraocularly, e.g., as described below.

Ophthalmic formulations can be delivered for topical administration, e.g., for administration as a liquid drop or an ointment, or for implantation, e.g., into an anterior chamber of the eye or the conjunctival sac. Liquid drops can be delivered using an eye dropper. When formulated for ocular delivery, the IL-17R binding protein can be present at 0.001-5%, e.g., 0.01-5%, 0.1-2% or 1%-5% concentration.

### Formulations

One or more therapeutic agent, alone or in combination with one or more chemotherapeutic agents, can be formulated with a pharmaceutically acceptable carrier for administration to a subject. In some embodiments, a therapeutic agent is formulated in combination with a mobilization factor, and optionally a chemotherapeutic agent. The active ingredients can be formulated alone (individually) for sequential administration or may be formulated together for concurrent administration.

The term “pharmaceutically acceptable carrier” as used herein means one or more compatible solid or liquid filler, diluents or encapsulating substances which are suitable for administration to a subject. The components of the pharmaceutical compositions also are capable of being commingled with each other, in a manner such that there is no interaction, which would substantially impair the desired pharmaceutical efficiency. Such preparations may routinely contain pharmaceutically acceptable concentrations of salt, buffering agents, preservatives, compatible carriers, adjuvants and optionally other therapeutic ingredients.

The compositions described herein may be administered as a free base or as a pharmaceutically acceptable salt. Such pharmacologically and pharmaceutically acceptable salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene sulphonic, and benzene sulphonic. Also, pharmaceutically acceptable salts can be prepared as alkaline metal or alkaline earth salts, such as sodium, potassium or calcium salts of the carboxylic acid group.

The pharmaceutical compositions also may comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

Suitable liquid or solid pharmaceutical preparation forms are, for example, aqueous or saline solutions for inhalation, microencapsulated, encochleated, coated onto microscopic gold particles, contained in liposomes (including pH-dependent release formulations), lipidoids, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of the compositions, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer, Science 249:1527-1533, 1990 and Langer and Tirrell, Nature, 2004 Apr. 1; 428(6982): 487-92.

The compositions may conveniently be presented in unit dosage form and may be prepared by any of the methods well known in the art of pharmacy. In certain embodiments, the composition that is administered is in powder or particulate form rather than as a solution. Examples of particulate forms contemplated as part of the invention are provided in U. S. 2002/0128225. In some embodiments, the compositions are administered in aerosol form. In other embodiments, the compositions may be in powder form for constitution with a suitable vehicle, e.g., sterile pyrogen-free water, before use.

In addition, the compositions described herein may be formulated as a depot preparation, time-release, delayed release or sustained release delivery system. Such systems can avoid repeated administrations of the compositions described herein, increasing convenience to the subject and the physician. Such long acting formulations may be formulated with suitable polymeric or hydrophobic materials (for example as an emulsion in an acceptable oil) or ion exchange resins, or as sparingly soluble derivatives, for example, as a sparingly soluble salt. Many types of release delivery systems are available and known to those of ordinary skill in the art. They include polymer based systems such as polylactic and polyglycolic acid, beta-glucan particles, polyanhydrides and polycaprolactone; nonpolymer systems that are lipids including sterols such as cholesterol, cholesterol esters and fatty acids, neutral fats such as mono-, di- and triglycerides or lipidoids; hydrogel release systems; silastic systems; peptide based systems; wax coatings, compressed tablets using conventional binders and excipients, partially fused implants and the like. In addition, a pump-based hardware delivery system can be used, some of which are adapted for implantation.

Controlled release can also be achieved with appropriate excipient materials that are biocompatible and biodegradable. These polymeric materials which effect slow release may be any suitable polymeric material for generating particles, including, but not limited to, nonbioerodable/non-biodegradable and bioerodable/biodegradable polymers. Such polymers have been described in great detail in the prior art and include, but are not limited to: beta-glucan particles, polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulfate sodium salt, poly(methyl methacrylate), poly(ethylmethacrylate), poly(butylmethacrylate), poly(isobutylmethacrylate), poly(hexylmethacrylate), poly(isodecylmethacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene poly(ethylene glycol), poly(ethylene oxide), poly(ethylene terephthalate), poly(vinyl alcohols), poly(vinyl acetate, poly vinyl chloride polystyrene, polyvinylpryrrolidone, hyaluronic acid, and chondroitin sulfate. In one embodiment the slow release polymer is a block copolymer, such as poly(ethylene glycol) (PEG)/poly(lactic-co-glycolic acid) (PLGA) block copolymer.

Examples of non-biodegradable polymers include ethylene vinyl acetate, poly(meth)acrylic acid, polyamides, copolymers and mixtures thereof.

Examples of biodegradable polymers include synthetic polymers, for example, beta-glucan particles, polymers of lactic acid and glycolic acid, polyanhydrides, poly(ortho)esters, polyurethanes, poly(butic acid), poly(valeric acid), poly(caprolactone), poly(hydroxybutyrate), poly(lactide-co-glycolide) and poly(lactide-co-caprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion. The foregoing materials may be used alone, as physical mixtures (blends), or as co-polymers. Preferred polymers are polyesters, polyanhydrides, polystyrenes and blends thereof.

Effective amounts of the compositions described herein are administered to a subject in need of such treatment. Effective amounts are those amounts, which will result in a desired improvement in the condition, disease or disorder or symptoms of the condition, disease or disorder.

Effective doses range from 1 ng/kg to 100 mg/kg body weight, or from 100 ng/kg to 50 mg/kg body weight, or from 1 μg/kg to 10 mg/kg body weight, depending upon the mode of administration. Alternatively, effective doses can range from 3 micrograms to 14 milligrams per 4 square centimeter area of cells. The absolute amount will depend upon a variety of factors (including whether the administration is in conjunction with other methods of treatment, the number of doses and individual patient parameters including age, physical condition, size and weight) and can be determined with routine experimentation. One useful dose that can be is the highest safe dose according to sound medical judgment.

The time between the delivery of the various active agents can be defined rationally by first principles of the kinetics, delivery, release, agent pharmacodynamics, agent pharmacokinetics, or any combination thereof. Alternatively, the time between the delivery of the various agents can be defined empirically by experiments to define when a maximal effect can be achieved.

### Mode of Administration

The mode of administration may be any medically acceptable mode including oral administration, sublingual administration, intranasal administration, intratracheal administration, inhalation, ocular administration, topical administration, transdermal administration, intradermal administration, rectal administration, vaginal administration, subcutaneous administration, intravenous administration, intramuscular administration, intraperitoneal administration, intrasternal, administration, or via transmucosal administration. In addition, modes of administration may be via an extracorporeal device and/or tissue-penetrating electro-magnetic device.

The particular mode selected will depend upon the particular active agents selected, the desired results, the particular condition being treated and the dosage required for therapeutic efficacy. The methods described herein, generally speaking, may be practiced using any mode of administration that is medically acceptable, for example, any mode that produces effective levels of inflammatory response alteration without causing clinically unacceptable adverse effects.

The compositions can be provided in different vessels, vehicles or formulations depending upon the disorder and mode of administration. For example, for oral application, the compositions can be administered as sublingual tablets, gums, mouth washes, toothpaste, candy, gels, films, etc.; for ocular application, as eye drops in eye droppers, eye ointments, eye gels, eye packs, as a coating on a contact lens or an intraocular lens, in contacts lens storage or cleansing solutions, etc.; for topical application, as lotions, ointments, gels, creams, sprays, tissues, swabs, wipes, etc.; for vaginal or rectal application, as an ointment, a tampon, a suppository, a mucoadhesive formulation, etc.

The compositions, may be administered by injection, e.g., by bolus injection or continuous infusion, via intravenous, subcutaneous, intramuscular, intraperitoneal, intrasternal routes. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. For oral administration, the compositions can be formulated readily by combining the compositions with pharmaceutically acceptable carriers well known in the art, e.g., as a sublingual tablet, a liquid formulation, or an oral gel.

For administration by inhalation, the compositions may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compositions and a suitable powder base such as lactose or starch. Medical devices for the inhalation of therapeutics are known in the art. In some embodiments the medical device is an inhaler. In other embodiments the medical device is a metered dose inhaler, diskhaler, Turbuhaler, diskus or a spacer. In certain of these embodiments the inhaler is a Spinhaler (Rhone-Poulenc Rorer, West Malling, Kent). Other medical devices are known in the art and include Inhale/Pfizer, Mannkind/Glaxo and Advanced Inhalation Research/Alkermes.

The compositions may also be formulated in rectal or vaginal compositions such as suppositories or retention enemas, e.g., containing conventional suppository bases such as cocoa butter or other glycerides.

### Production of Antibodies

Exemplary IL-17 cytokine antagonists are antibodies, e.g., antibodies that bind to an IL-17 cytokine receptor, such as IL-17RA, IL-17RB, IL-17RC, IL-17RD, or IL-17RE or antibodies that bind to an IL-17 cytokine, e.g., IL-17A, IL-17B, IL-17C, IL-17D, IL-17E, or IL-17F. As used herein, the term “antibody” refers to a protein that includes at least one immunoglobulin variable region. For example, an antibody can include a heavy chain variable region (VH), and a light chain variable region (VL). In another example, an antibody includes two VH regions and two VL regions. The term “antibody” encompasses antigen-binding fragments of antibodies (e.g., single chain antibodies, Fab fragments, F(ab′)₂ fragments, Fd fragments, Fv fragments, and dAb fragments) as well as complete antibodies, e.g., intact immunoglobulins of types IgA, IgG, IgE, IgD, IgM (as well as subtypes and modified versions thereof). Still other antibodies only include a single immunoglobulin variable domain. See, e.g., Janssens et al., Proc. Natl. Acad. Sci. USA, 103(41):15130-5 (2006).

Each VH and VL is typically composed of three “complementarity determining regions” (“CDR”) and four “framework regions” (FR), arranged from amino-terminus to carboxyl-terminus in the following order: FR1, CDR1, FR2, CDR2, FR3, CDR3, FR4. The extent of the FRs and CDRs has been precisely defined (see, Kabat, E. A., et al. (1991) Sequences of Proteins of Immunological Interest, Fifth Edition, U. S. Department of Health and Human Services, NIH Publication No. 91-3242; and Chothia, C. et al. (1987) J. Mol. Biol. 196:901-917). Kabat definitions are used herein. The canonical structures of hypervariable loops of an immunoglobulin variable can be inferred from its sequence, as described in Chothia et al. (1992) J. Mol. Biol. 227:799-817; Tomlinson et al. (1992) J. Mol. Biol. 227:776-798); and Tomlinson et al. (1995) EMBO J. 14(18):4628-38.

An exemplary antibody binds specifically to an IL-17 cytokine or an IL-17 cytokine receptor, e.g., with a binding affinity 10⁶ M or greater, preferably 10⁷ M or greater, more preferably 10⁸ M or greater, and most preferably 10⁹ M or greater. The binding affinity of an antibody can be readily determined by one of ordinary skill in the art, for example, by Scatchard analysis. An exemplary antibody may also have an EC50 of less than 100 nM, 20 nM, or 5 nM. Further, an exemplary antibody can interfere with binding of an IL-17 cytokine and an IL-17 cytokine receptor, e.g., binding of IL-17A to IL-17RA or IL-17RC, or binding of IL-17F to IL-17RA or IL-17RC.

A specific antibody does not significantly cross-react with unrelated polypeptide molecules, for example, if they detect a desired polypeptide(s), but not other cellular polypeptides using a standard Western blot analysis. In some embodiments, the antibody is specific for one IL-17 cytokine or one IL-17 receptor relative to others, e.g., the antibody preferentially binds to one particular IL-17 cytokine or receptor by a factor of at least 10, 100, or 1000.

In one embodiment, the antibody binds to IL-17RA, e.g., the D1 or D2 domain of IL-17RA. For example the antibody binds to an epitope that includes one or more amino acids within amino acids 22-36, amino acids 83-96, amino acids 118-147, amino acids 152-179, or amino acids 256-271 of IL-17RA (SEQ ID NO:14), e.g., one or more amino acids, e.g., at least two or three amino acids within: Thr25-Trp31, Leu86-Arg93, or Cys259-Arg265 of SEQ ID NO:14. For example, the antibody reduces binding between IL-17RA and an IL-17 cytokine, e.g., IL-17A or IL-17F, e.g., by at least 100, 200, 500, 1000, or 5000 fold.

In another embodiment, the antibody binds to IL-17RB, e.g., to an epitope that includes one or more amino acids within amino acids 25-39, amino acids 86-100, amino acids 126-155, amino acids 160-187, or amino acids 254-269 of IL-17RB (SEQ ID NO:15) and/or amino acids 32-44 (e.g., 38-44), 82-98 (e.g., 88-98), and 252-269 (e.g., 256-263) of SEQ ID NO:15. In another embodiment, the antibody binds to IL-17RC, e.g., to an epitope that includes one or more amino acids within amino acids 15-30, amino acids 70-84, amino acids 96-124, amino acids 129-156, or amino acids 227-237 of IL-17RC (SEQ ID NO:16) and/or amino acids 24-35, 78-91, and 248-257 of SEQ ID NO:16.

Polyclonal antibodies to a polypeptide can be prepared using known methods. See, for example, Green et al., “Production of Polyclonal Antisera,” in Immunochemical Protocols (Manson, ed.) (Humana Press 1992). Monoclonal antibodies can be generated. Rodent monoclonal antibodies to specific antigens may be obtained by methods known to those skilled in the art (See, for example, Kohler et al., Nature 256:495 (1975); Coligan et al. (eds.), Current Protocols in Immunology (John Wiley & Sons 1991); Picksley et al., “Production of monoclonal antibodies against proteins expressed in E. coli,” in DNA Cloning 2: Expression Systems, 2^{nd} Edition, Glover et al. (eds.) (Oxford University Press 1995)).

For example, monoclonal antibodies can be obtained by injecting mice with a composition including the polypeptide, verifying the presence of antibody production by removing a serum sample, removing the spleen to obtain B-lymphocytes, fusing the B-lymphocytes with myeloma cells to produce hybridomas, cloning the hybridomas, selecting positive clones that produce antibodies to the antigen, culturing the clones that produce antibodies to the antigen, and isolating the antibodies from the hybridoma cultures.

Human antibodies to the polypeptide can also be derived. Human monoclonal antibodies are obtained from transgenic mice that have been engineered to produce specific human antibodies in response to antigenic challenge. In this technique, elements of the human heavy and light chain locus are introduced into strains of mice derived from embryonic stem cell lines that contain targeted disruptions of the endogenous heavy chain and light chain loci. The transgenic mice can synthesize human antibodies specific for human antigens, and the mice can be used to produce human antibody-secreting hybridomas. Methods for obtaining human antibodies from transgenic mice are described, for example, by Green et al., Nature Genet. 7:13 (1994), Lonberg et al., Nature 368:856 (1994), and Taylor et al., Int. Immun. 6:579 (1994).

Monoclonal antibodies can be isolated and purified from hybridoma cultures by a variety of well-established techniques. Such isolation techniques include affinity chromatography with Protein-A Sepharose, size-exclusion chromatography, and ion-exchange chromatography (see, for example, Coligan; Baines et al., “Purification of Immunoglobulin G (IgG),” in Methods in Molecular Biology, (The Humana Press, Inc. 1992)).

An antibody can be a “humanized” monoclonal antibody. Humanized monoclonal antibodies are produced by transferring mouse complementary determining regions from heavy and light variable chains of the mouse immunoglobulin into a human variable domain. Typical residues of human antibodies are then substituted in the framework regions of the murine counterparts. The use of antibody components derived from humanized monoclonal antibodies obviates potential problems associated with the immunogenicity of murine constant regions. General techniques for cloning murine immunoglobulin variable domains are described, for example, by Orlandi et al., Proc. Nat'l Acad. Sci. USA 86:3833 (1989). Techniques for producing humanized monoclonal antibodies are described, for example, by Jones et al., Nature 321:522 (1986); Carter et al., Proc. Nat'l Acad. Sci. USA 89:4285 (1992); Sandhu, Crit. Rev. Biotech. 12:437 (1992); Singer et al., J. Immun. 150:2844 (1993); Sudhir (ed.), Antibody Engineering Protocols (Humana Press, Inc. 1995); Kelley, “Engineering Therapeutic Antibodies,” in Protein Engineering: Principles and Practice, Cleland et al. (eds.) (John Wiley & Sons, Inc. 1996); and by Queen et al., U. S. Pat. No. 5,693,762.

A variety of assays known to those skilled in the art can be utilized to detect antibodies which specifically bind to a polypeptide. Exemplary assays are described in detail in Antibodies: A Laboratory Manual, Harlow and Lane (Eds.), Cold Spring Harbor Laboratory Press, 1988. Representative examples of such assays include: radioimmunoassays, radioimmunoprecipitations, enzyme-linked immunosorbent assays (ELISA), dot blot or Western blot assays, inhibition or competition assays, sandwich assays, and surface plasmon resonance.

### F_{C} and Other Fusion Proteins

A protein disclosed herein, e.g. a IL-17R binding protein, can be associated with a heterologous domain, such as a constant domain of an immunoglobulin or the Fc region of an immunoglobulin, a serum albumin, or a serum albumin binding domain. For example, at least one IL-17 polypeptide sequence and one or more constant domains of an Fc region can be components of the same polypeptide chain, and can for example be joined by a linker. An exemplary Fc region is from a human IgG, e.g., IgG1, IgG2, IgG3, or IgG4. The heterologous polypeptide can include all or a portion of the CH2 domain, the CH3 domain, and/or a hinge region, of an immunoglobulin. The heterologous polypeptide can be connected by a linker, e.g., a flexible linker.

Fragments of an Fc region can also be used, as can Fc muteins. For example, certain residues within the hinge region of an F_{c} region are critical for high affinity binding to F_{c}γRI. Canfield and Morrison (1991) J. Exp. Med. 173:1483) reported that Leu234 and Leu235 are critical to high affinity binding of IgG₃ to F_{c}γRI present on U937 cells. Similar results were obtained by Lund et al. (1991) J. Immunol. 147:2657. Such mutations, alone or in combination, can be made in an IgG1 Fc region to decrease the affinity of IgG1 for FcR. Other Fc muteins that effect Fc binding, antibody-dependent cell mediated cytotoxicity (ADCC) and complement dependent cytotoxicity (CDC) are described in Shields et al. (2001) J. Biol. Chem. 276(9):6591 and US 2004/0132101.

### Additional Uses

A binding protein described herein (e.g., an IL-17R binding protein or antibody described herein) can be labeled directly or indirectly with a moiety that is a label or produces a signal, e.g., an enzyme, a radiolabel, an epitope, or a fluorescent protein (such as green fluorescent protein). The binding protein can be contacted to a sample or to cells to determine if a receptor is present in the sample or on the cells, e.g., using standard immunoblotting, immunofluorescence, enzyme immunoassay (EIA), radioimmunoassay (RIA), fluorescence energy transfer, Western blot, and other diagnostic and detection techniques.

The binding protein can also be labeled for in vivo detection and administered to a subject. The subject can be imaged, e.g., by NMR or other tomographic means. For example, the binding agent can be labeled with a radiolabel such as 131I, 111In, 123I, 99 mTc, 32P, 125I, 3H, 14C, and 188Rh, fluorescent labels such as fluorescein and rhodamine, nuclear magnetic resonance active labels, positron emitting isotopes detectable by a positron emission tomography (“PET”) scanner, chemiluminescers such as luciferin, and enzymatic markers such as peroxidase or phosphatase. The binding protein can be labeled with a contrast agent such as paramagnetic agents and ferromagnetic or superparamagnetic (which primarily alter T2 response)

A binding protein can also be used to purify cells which express the receptor to which it binds. For example, the binding protein can be coupled to an immobilized support (e.g., magnetic beads or a column matrix) and contacted to cells which may express the receptor. The support can be washed, e.g., with a physiological buffer, and the cells can be recovered from the support.

A binding protein can also be used to purify soluble forms of the receptor to which it binds. For example, samples containing the soluble receptor can be contacted to immobilized binding protein and then, e.g., after washing, can be recovered from the immobilized binding protein.

A binding protein that binds to an IL-17 receptor can also be used to delivery a toxin or cytotoxic effect to an IL-17 receptor expressing cell. For example, the binding protein can be associated with (e.g., covalently) with a toxin or may be conjugated to a therapeutic moiety such as a cytotoxin, a therapeutic agent or a radioactive metal ion. A cytotoxin or cytotoxic agent includes any agent that is detrimental to cells, including another protein, e.g., a toxin such as abrin, ricin A, pseudomonas exotoxin, or diphtheria toxin, or an Fc domain competent to recruit an ADCC or complement mediated cytotoxic response. Other toxins that can be associated with the binding protein include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine and lomustine, cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin, bleomycin, mithramycin, and anthramycin), and anti-mitotic agents (e.g., vincristine and vinblastine).

For example, the binding protein can be coupled to a radioactive isotope such as an α, β, or γ-emitter. Examples of radioactive isotopes include iodine (¹³¹I or ¹²⁵I), yttrium (⁹⁰Y), lutetium (¹⁷⁷Lu), actinium (²²⁵Ac), praseodymium, or bismuth (²¹²Bi or ²¹³Bi). The binding protein can be coupled to a biological protein, a molecule of plant or bacterial origin (or derivative thereof), e.g., a maytansinoid (e.g., maytansinol, an analog thereof or DM1), as well as a taxane (e.g., taxol or taxotere), or a calicheamicin. Examples of maytansinol analogues include those having a modified aromatic ring (e.g., C-19-decloro, C-20-demethoxy, C-20-acyloxy) and those having modifications at other positions (e.g., C-9-CH, C-14-alkoxymethyl, C-14-hydroxymethyl or aceloxymethyl, C-15-hydroxy/acyloxy, C-15-methoxy, C-18-N-demethyl, 4,5-deoxy). Maytansinol and maytansinol analogues are described, for example, in U. S. Pat. No. 6,333,410. Maytansinol can be coupled using, e.g., an N-succinimidyl 3-(2-pyridyldithio)proprionate (also known as N-succinimidyl 4-(2-pyridyldithio)pentanoate or SPP), 4-succinimidyl-oxycarbonyl-a-(2-pyridyldithio)-toluene (SMPT), N-succinimidyl-3-(2-pyridyldithio)butyrate (SDPB), 2-iminothiolane, or S-acetylsuccinic anhydride.

The entire disclosure of each of the patent documents and scientific articles referred to herein, and those patent documents and scientific articles cited thereby, is expressly incorporated by reference herein for all purposes.

### EXAMPLES

### Example 1

### IL-17RA-IL-17F Complex Expression and Crystallization

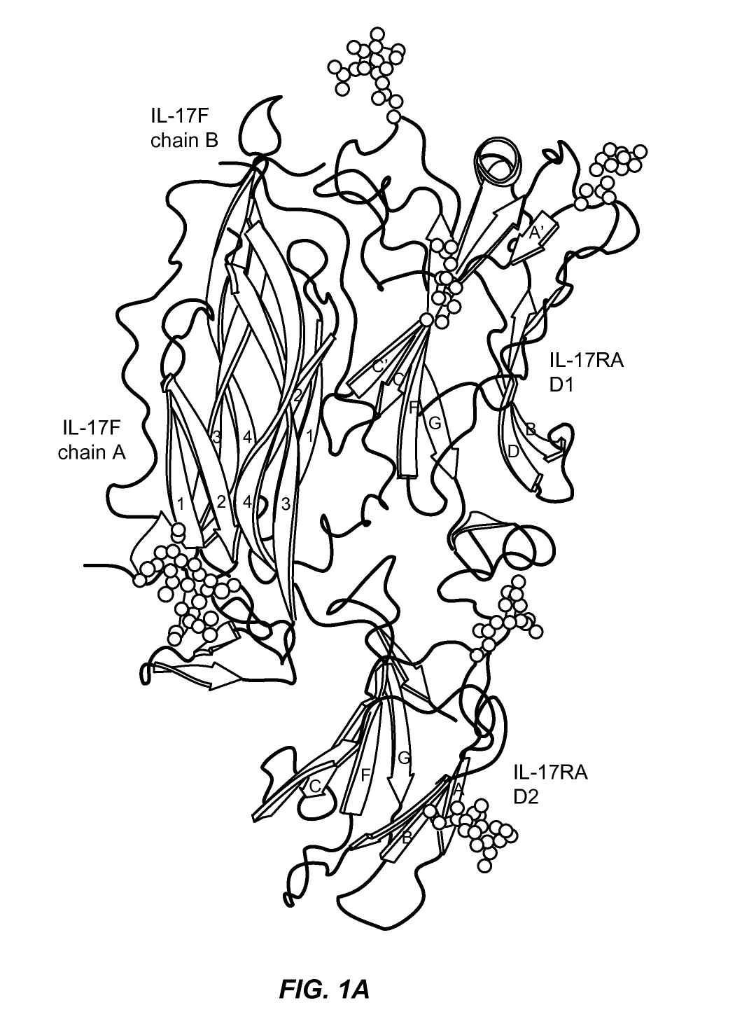

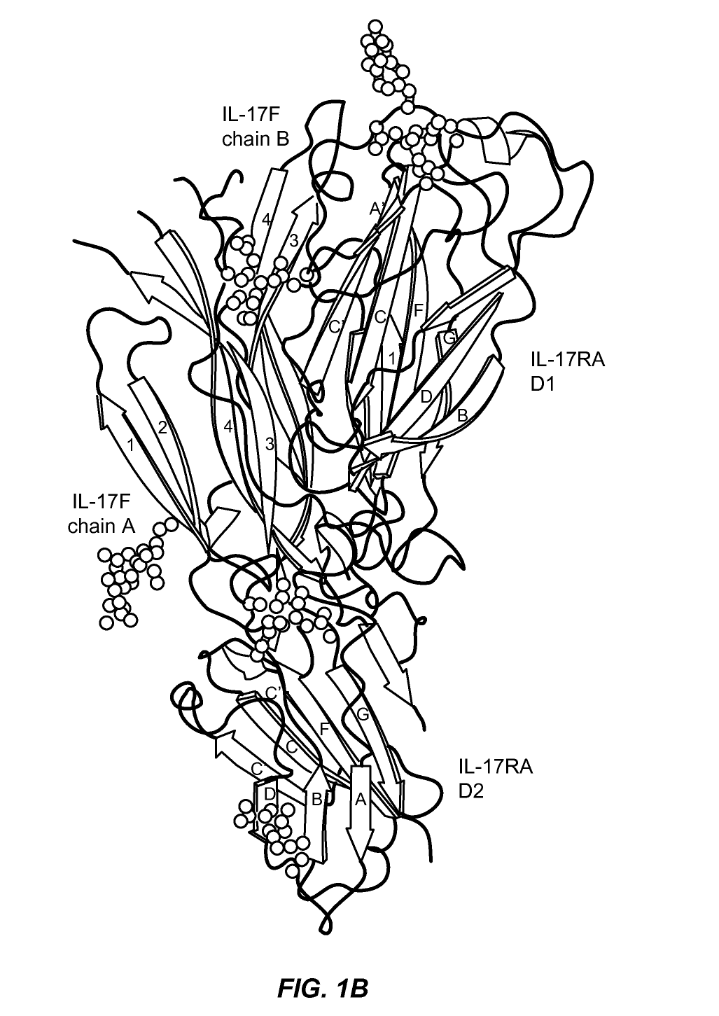

We determined the crystal structure of IL-17RA bound to IL-17F at 3.3 Å resolution using single isomorphous replacement with anomalous scattering (SIRAS) phasing (Table 1). We expressed IL-17F from baculovirus, and the IL-17RA extracellular domain (ECD) using 293S GnTI-cells. To facilitate crystallization, the complex was methylated, and the heavily glycosylated receptor ECD was ‘shaved’ with endoglycosidase H prior to crystallization to improve homogeneity, leaving one GlcNAc residue at each of the Asn-linked glycosylation sites (FIG. 1). Biochemically the shaved and unshaved complexes behaved identically. By gel filtration, mixtures of IL-17F or IL-17A with IL-17RA ECD resulted in co-elution of complexes with 2:2 (2 receptors+1 IL-17 dimer) and 1:2 (1 receptor+1 IL-17 dimer) stoichiometries, with the major species being the 1:2. The 2:2 was only detected at high protein concentrations, whereas at lower concentrations the 1:2 predominated even in the presence of excess IL-17RA. The crystals contained one IL-17RA bound to one IL-17F homodimer (FIG. 1). As discussed below, this ‘partial’ signaling complex may, in fact, be the biologically relevant form of the IL-17RA-IL-17F and IL-17RA-IL-17A complexes.

### Example 2

### IL-17RA-IL-17F Complex Overall Structure

The IL-17RA ectodomain is composed of two unusual FnIII domain modules joined by an 18-amino acid linker (FIG. 1). Although not apparent from the sequence, the IL-17RA structure is reminiscent of hematopoietic cytokine receptors in that it contains tandem b-sandwich domains; however, the domains themselves contain some substantial deviations from canonical FnIII folds, and the manner of ligand interaction is entirely distinct from other cytokine receptors. Residues 2-272 of the predicted 286 ectodomain residues (where residue 1 is the first amino acid of the mature peptide, as shown in SEQ ID NO:14) were modeled into continuous electron density for the receptor chain and five of the potential seven N-linked glycans were clearly visualized. The first FnIII domain (D1) has an additional 40 amino acid N-terminal extension that forms a unique fold. The chain makes a hairpin-like turn bridged by a disulfide bond (Cys12-Cys19), and the second strand of the turn forms a β-strand (A′) that extends the FnIII β-sheet and then wraps around the face of the D1 domain, disulfide bonding with the C′ strand Cys95, before passing over the domain to start the A-strand of the FnIII domain. The interdomain linker region contains a short helix and is stabilized by an internal disulfide bond (Cys154-Cys165). The second FnIII domain (D2) has two atypical disulfide bonds, one linking the C-C′ loop (Cys214) to the D-F loop (Cys245) and a second within the F-G loop (Cys259-Cys263). We predict that a third disulfide bond exists between F-G loop (Cys246) and C-terminus of the G-strand (Cys272), similar to that observed in class-II cytokine receptors (21), however this bond is not well defined in the current electron density map.

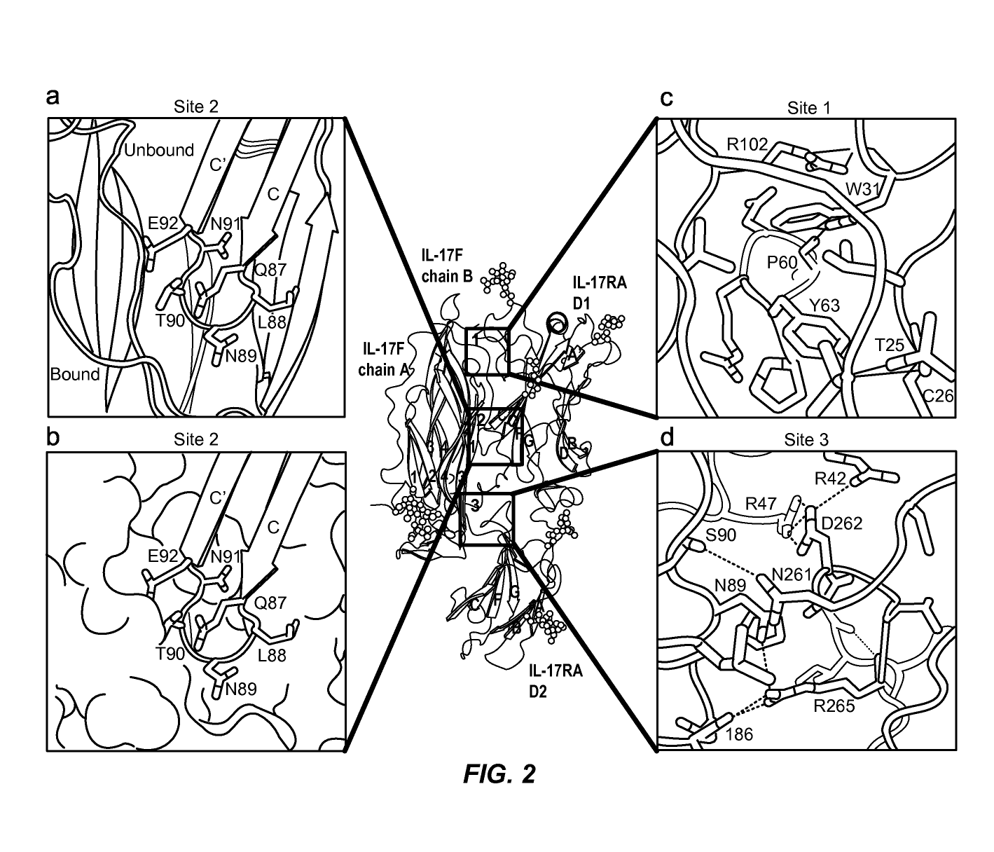

While the core structure of the IL-17RA-bound IL-17F molecule was essentially unchanged compared to that of the unliganded form of IL-17F (7), peripheral strands and loops underwent structural accommodations to facilitate binding to IL-17RA. The conformation observed in the unliganded IL-17F structure could not be maintained in the IL-17RA-bound state, as it would generate steric clashes with the N-terminal coil region of the receptor. Each IL-17F monomer is composed of two pairs of anti-parallel β-sheets (strands 1-4) with the second and fourth strands connected by two disulfide bonds in a manner homologous to cysteine-knot family proteins. There is a 50 amino acid N-terminal extension of which residues 29-42 run parallel to strands 3 and 4 of the second IL-17F protomer. This coil region is stabilized by numerous interactions, including several hydrogen bonds with the adjacent strands. In the IL-17RA-bound IL-17F conformation this region (residues 33-42) moves out to open up the binding pocket and interact with the receptor (FIG. 2A). The first 24 amino acids of each IL-17F chain, and residues 105-109 from the 3-4 loop on one IL-17F protomer, could not be modeled. In the unliganded IL-17F structure Cys17 forms a disulfide bond with Cys107 at the tip of the 3-4 loop on the adjacent IL-17F chain. These interchain disulfide bonds were not modeled, but were present as the protein behaved as a disulfide-linked dimer on SDS-PAGE.

### Example 3

### IL-17RA-IL-17F Binding Interface

The overall binding mode of IL-17F to IL-17RA, in which both receptor FnIII domains bind in a ‘side-on’ orientation and use edge strands to insert into a crevasse formed at the dimeric interface of the ligand, is unlike other cytokine or growth factor receptor complexes. IL-17RA forms an extensive binding interface with IL-17F, burying ˜2200 Å² of surface area; ˜70% of this buried surface area is mediated by the IL-17RA D1 domain. There are three major interaction sites at the binding interface (FIG. 2). Site 1 is formed between the N-terminal extension of IL-17RA (Thr25-Trp31 of SEQ ID NO:14) and the 1-2 loop (Pro60-Tyr63) plus the C-terminal region of strand 3 (Val100, Arg102) of IL-17F chain B; this interaction buries ˜330 Å² (FIG. 2C). Trp31 of the receptor is buried in the center of this binding site; the main-chain O forms hydrogen bonds with Arg102 and the side chain forms hydrogen bonds with Pro60. Two additional hydrogen bonds are formed between IL-17RA Thr25 and Cys26 and IL-17F Tyr63. Site 2 is the most prominent interface feature of the complex, and is composed of the IL-17RA D1 C′-C loop (Leu86-Arg93 of SEQ ID NO:14) which slots into a deep binding-pocket flanked by the N-terminal extension and strand 2 of IL-17F chain B and strand 3 of IL-17F chain A; this interaction buries almost 550 Å² (FIG. 2A,B). This 8-amino acid IL-17RA loop forms extensive hydrophobic and polar interactions with both chains of IL-17F including a potential salt bridge between IL-17RA Glu92 and IL-17F chain B Arg37, and a hydrogen bond between the main-chain O of IL-17RA Asn89 and IL-17F chain A Asn95. Site 3, which encompasses ˜410 Å² of buried surface area (BSA), is formed between the IL-17RA D2 F-G loop (Cys259-Arg265) and the C-terminal regions of stands 3 and 4 of IL-17F chain A, and the N-terminal extension of IL-17F chain B (FIG. 2D). Site 3 is rich in charged interactions with nine potential hydrogen bonds and a salt bridge between IL-17RA Asp262 and IL-17F chain B Arg47. Overall the interface is extensive and is composed of numerous specific contacts. It is envisaged that an analogous binding mode will be used by other IL-17 receptor-cytokine pairs, given the sequence conservation of contact residues (discussed below). However, a greater bond-network and/or shape complementarity may be employed in the higher affinity complexes.

### Example 4

### Heterodimeric Receptor Complex Formation

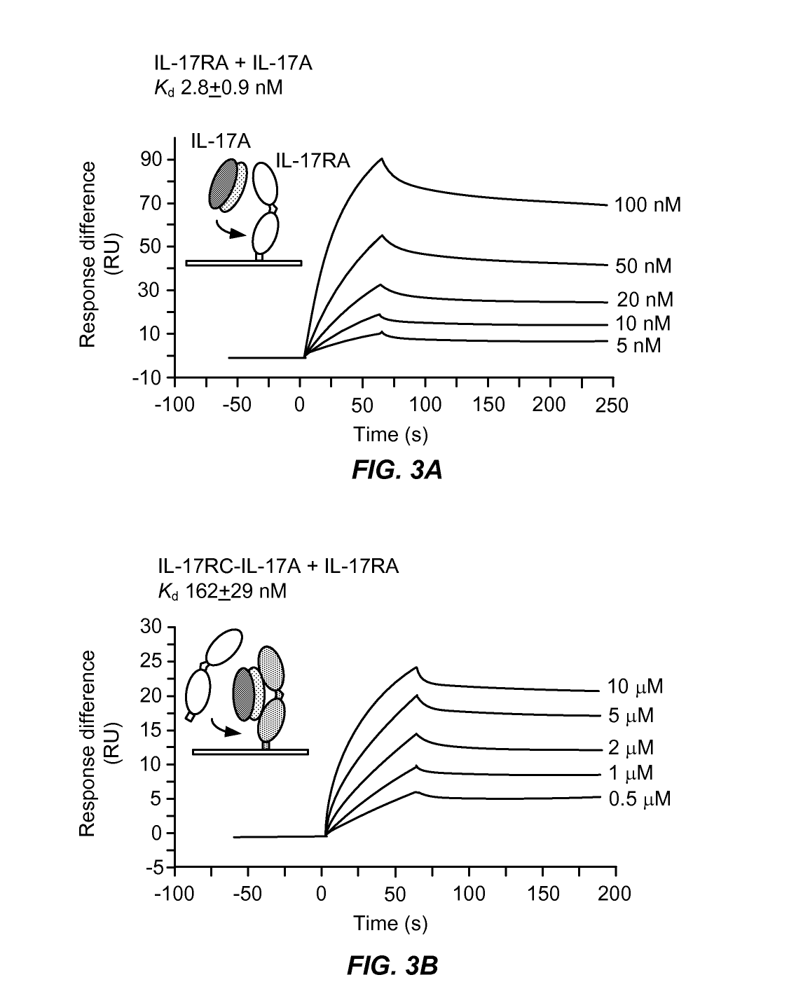

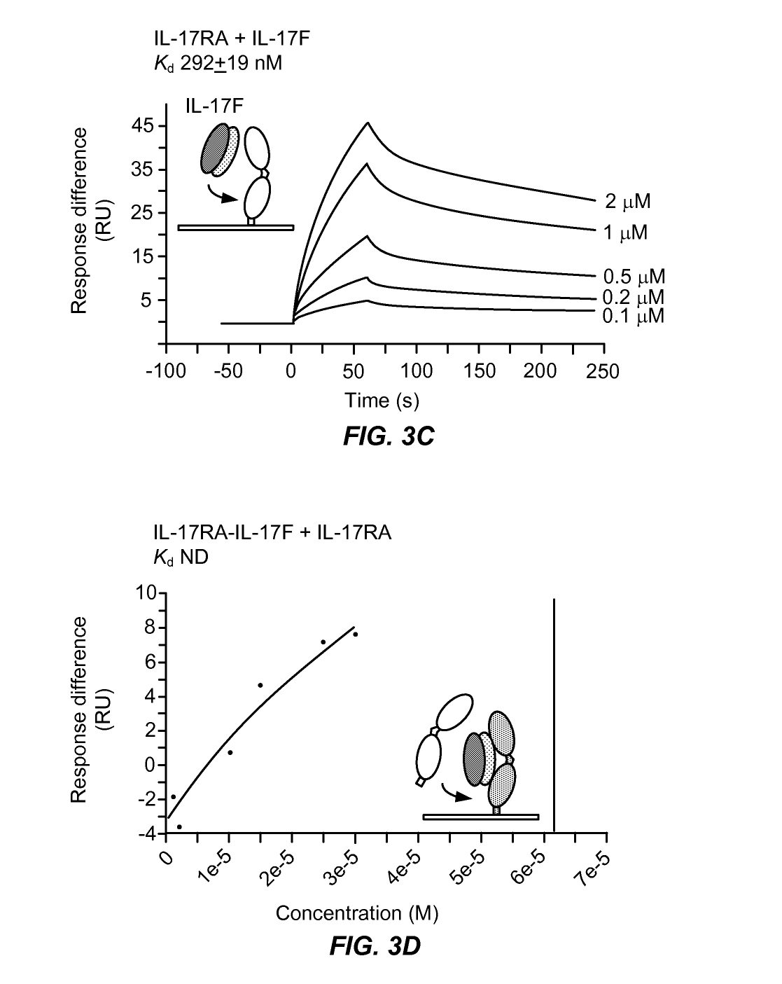

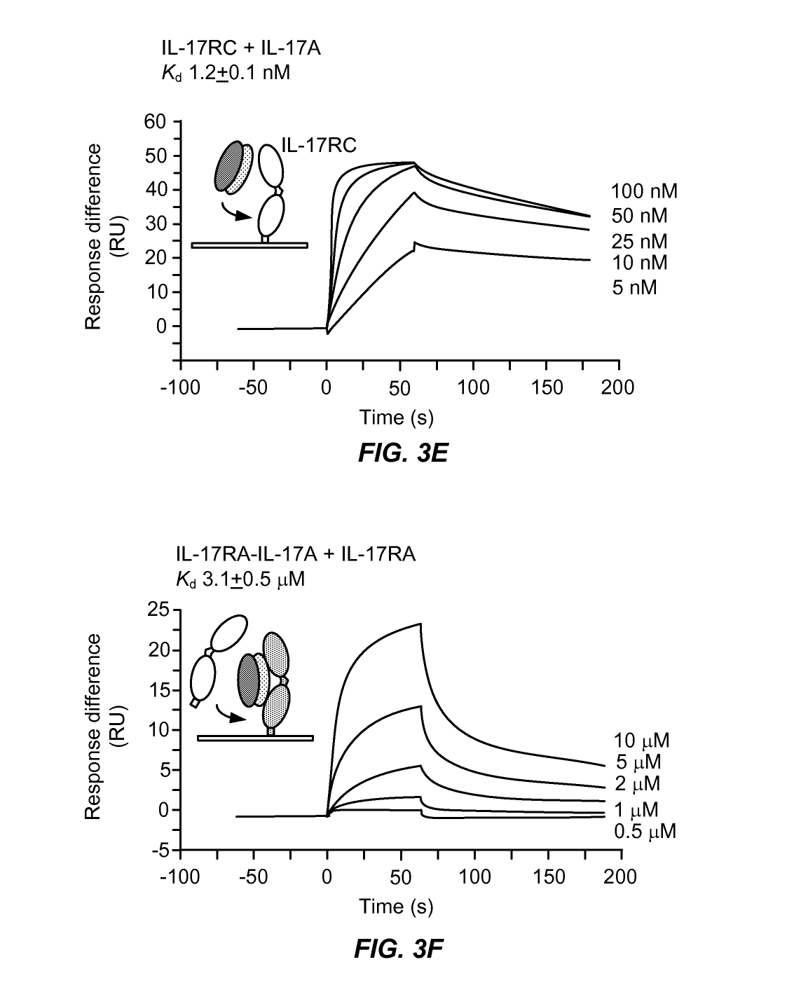

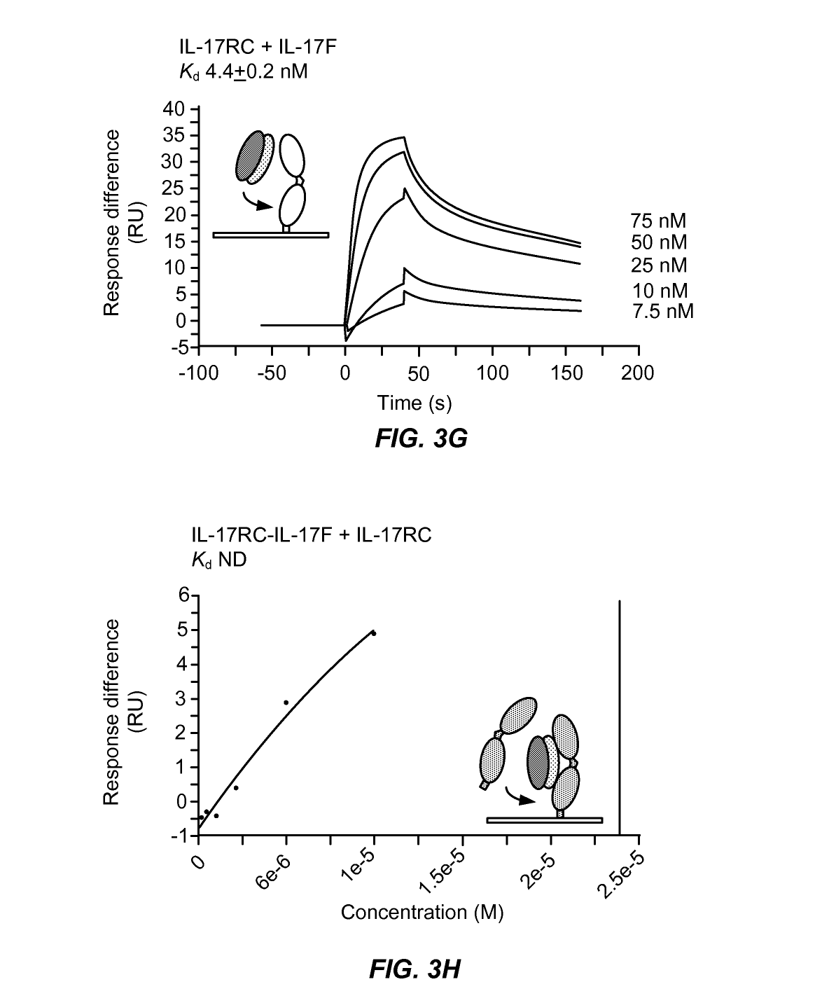

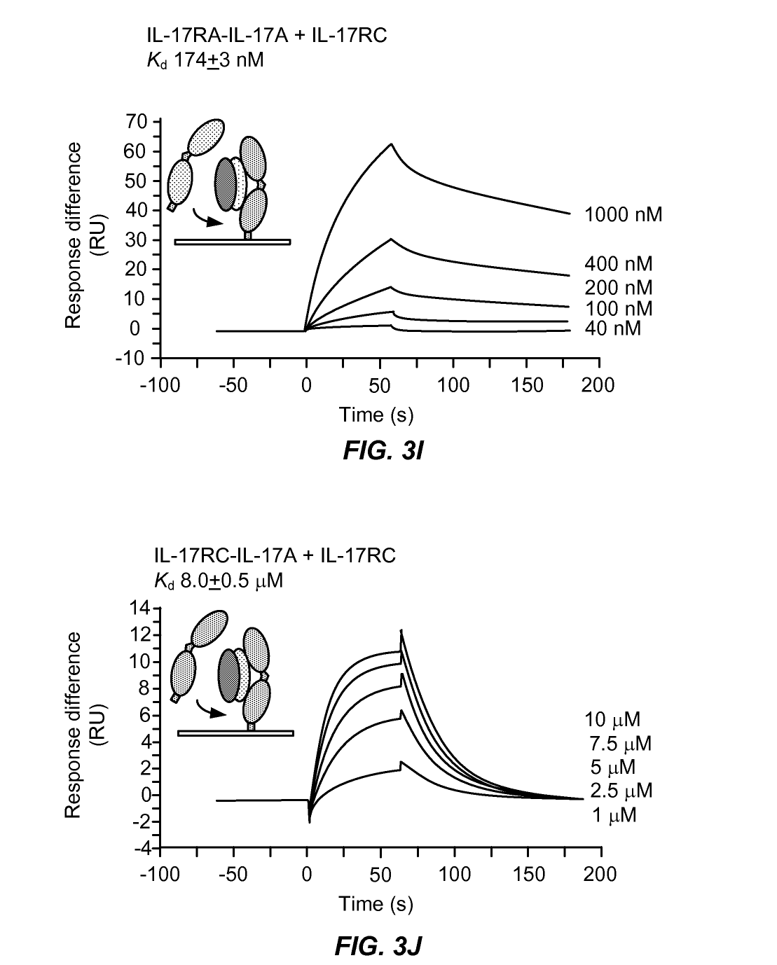

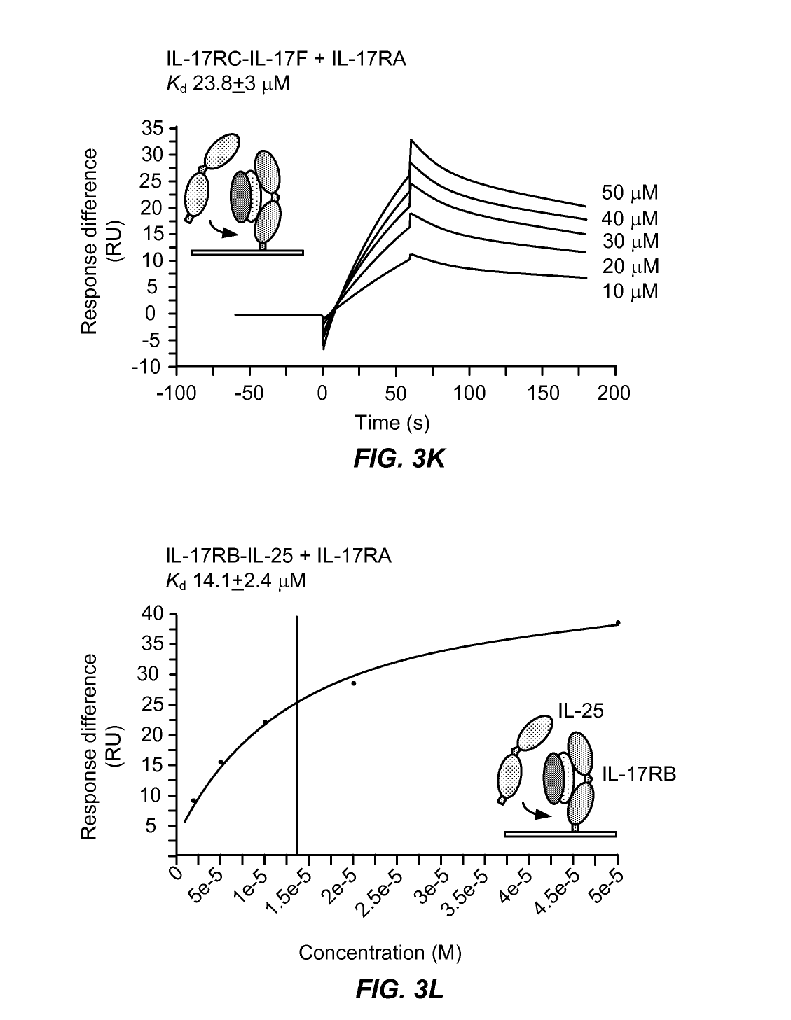

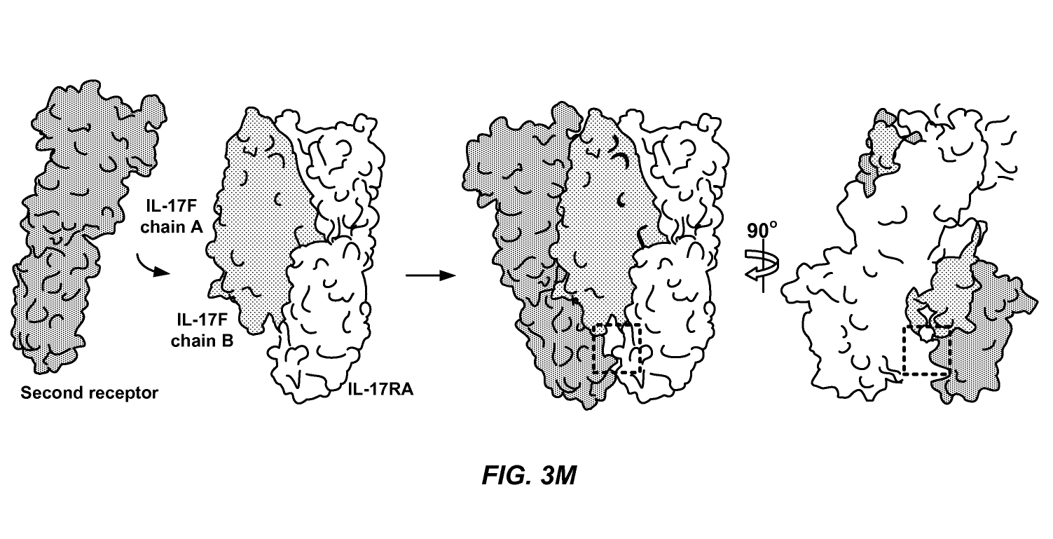

The stoichiometries of the receptor complexes remain to be fully elucidated (6), but the asymmetric IL-17RA-IL-17F complex hints at a preference for heterodimerization with a second, different receptor. We therefore investigated the mechanism by which a homodimeric cytokine could possibly coordinate two different receptors. Both IL-17RA and IL-17RC can bind independently to IL-17A and IL-17F, but both receptors are necessary for signaling (9, 10, 22). To further understand how the signaling complex is formed we devised a surface plasmon resonance (SPR) strategy using soluble proteins to measure the affinities of both the homodimeric and heteromeric receptor complexes for cytokine in vitro. Whilst others have reported the binding affinities of IL-17RA and IL-17RC for IL-17A and IL-17F (7, 22), we considered it pertinent to assess the binding affinity of the second receptor-binding site. The strategy was to immobilize one receptor on the SPR chip at a low coupling density in order to minimize possible homo-dimerization (e.g. cross-linking) of the receptors on the chip. The dimeric IL-17 cytokine was then captured by this receptor so that each receptor would be bound to one dimeric IL-17 ligand, leaving an exposed and accessible second receptor-binding site. The second receptor was subsequently passed over the preformed receptor-cytokine complexes to measure the affinity of the second receptor-binding event. In this fashion, the complex was assembled in a stepwise manner and each of the binding affinities was measured (FIG. 3). IL-17A bound to both IL-17RA (2.8±0.9 nM) and IL-17RC (1.2±0.1 nM) with high affinity. Once IL-17A was bound by one IL-17RA molecule, the binding affinity for a second IL-17RA was reduced to 3.1±0.5 μM whereas the IL-17RC affinity for this second binding site was 174±3 nM. If the IL-17A was originally captured by IL-17RC, a second IL-17RA bound to the existing IL-17RC-IL-17A complex with 162±29 nM affinity; the binding affinity of a second IL-17RC to existing IL-17RC-IL-17A complex was only 8.0±0.5 μM.

A similar pattern was observed for IL-17F, which has a higher affinity for IL-17RC (4.4±0.2 nM) compared to IL-17RA (292±19 nM). Given the divergent affinities it seems likely that IL-17F would be initially captured by IL-17RC; once bound, the affinity of IL-17RA for the IL-17RC-IL-17F complex was 23.8±3 μM. In contrast, the binding affinity of IL-17RA and IL-17RC for preformed IL-17RA-IL-17F and IL-17RC-IL-17F complexes, respectively, was so weak that it could not be accurately calculated over the concentration range used for these experiments. Thus, these findings clearly show that engagement of IL-17RA or IL-17RC by IL-17A or IL-17F encourages a preference for the second receptor-binding site to engage a different receptor and thereby to form the heterodimeric receptor complex.

IL-17RA has been implicated in IL-17E (also known as IL-25) signaling together with IL-17RB (23). IL-25 promotes Th2 inflammatory responses and shares approximately ˜20% identity with IL-17A and IL-17F. Binding experiments have demonstrated that whilst IL-25 binds to IL-17RB with high affinity, it has no apparent affinity for IL-17RA (23-25). We hypothesized that IL-17RA may only bind IL-25 once IL-25 is captured by IL-17RB. To test this hypothesis, we immobilized IL-17RB on an SPR chip, captured IL-25 and measured the affinity of IL-17RA for the IL-17RB-IL-25 complex. Supporting our hypothesis, IL-17RA bound to the IL-17RB-IL-25 complex with 14.1±2.4 μM affinity (FIG. 3). At concentrations up to 50 μM, no interaction could be observed between IL-17RA and IL-25, or between the IL-17RB-IL-25 complex and a second IL-17RB molecule. Together with the IL-17A and IL-17F binding data, these results indicate that the formation of the heteromeric complex may be mediated by allostery and/or an interaction between the receptors.

To further address this concept we modeled a second IL-17RA molecule to form the hypothetical 2:2 receptor-cytokine complex (FIG. 3B). Assuming that the second receptor binds in an identical fashion to the first, the base of IL-17RA D2 would come into very close proximity with the D2 of the second IL-17RA (FIG. 3B, dashed box). In the case of two IL-17RA molecules bound to IL-17F, His212 on the C-C′ loop of one IL-17RA would clash with the second IL-17RA His212. This potential interaction site may allow the receptors to regulate their pairing. Steric clashes may cause reduced affinity for a second identical receptor, or favorable receptor-receptor interactions may stabilize heteromeric receptor complexes. We do not rule out the possibility that homodimeric receptor complexes could form on cells under certain conditions, however, our data argues that receptor heterodimers will likely be the predominant signaling species.

### Example 5

### IL-17RA Functions as a Common Receptor

IL-17RA binds to IL-17A with ˜100-fold higher affinity than IL-17F. IL-17A and IL-17F share ˜50% identity, and mapping the conserved residues onto the structure of IL-17F reveals a horseshoe-shaped ring of variable residues around the receptor-binding pocket (FIG. 4). The majority of the IL-17RA C′-C loop interactions are formed with residues that differ between the IL-17A and IL-17F molecules whereas the N-terminal region and IL-17RA D2 F-G loop interactions involve predominately conserved residues. We reported here that the extracellular region of IL-17RA can also bind to the IL-17RB-IL-25 complex, and it was recently shown that IL-17RD can interact with IL-17RA to mediate IL-17A signaling (26). Given this association of IL-17RA with diverse IL-17 family members we speculate that IL-17RA may act as a shared receptor analogous to those utilized in class I cytokine receptor complexes (27). To investigate this possibility, we mapped the residues conserved among all IL-17 family members onto the IL-17F surface. Analyzing the location of these residues in the IL-17RA-IL-17F complex, it seems plausible that IL-17RA contacts these conserved residues with the N-terminal region of the D1 domain and the F-G loop of the D2 domain (FIG. 4C). In contrast, IL-17RA may modulate specificity for each cytokine by contacting non-conserved cytokine residues with the C-C′ loop (FIG. 4C). Collectively, then, IL-17RA appears to use a strategy of cross-reactivity based on a subset of conserved contacts, amongst a background of distinct contacts, with several different IL-17 cytokines. This is similar to the strategy utilized by the shared p75 receptor for recognition of different neurotrophin ligands²⁸, and stands in contrast to the mechanism used for cross-reactivity by, for example, gp130 and g_{c} chain, which form largely disparate molecular interactions with different four-helix cytokines (27).

### Example 6

### Receptor Binding Modes of Cysteine-Knot Growth Factors

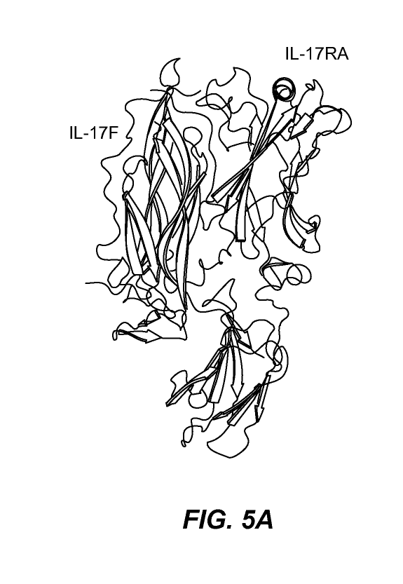

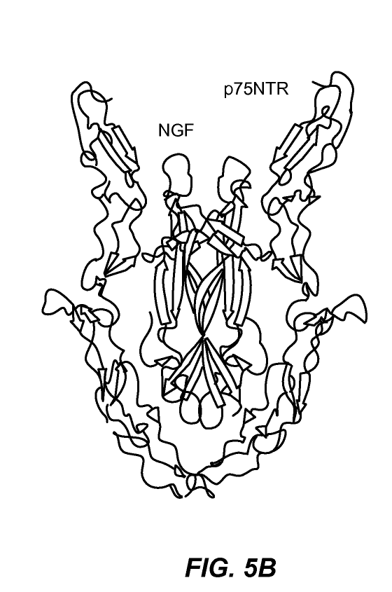

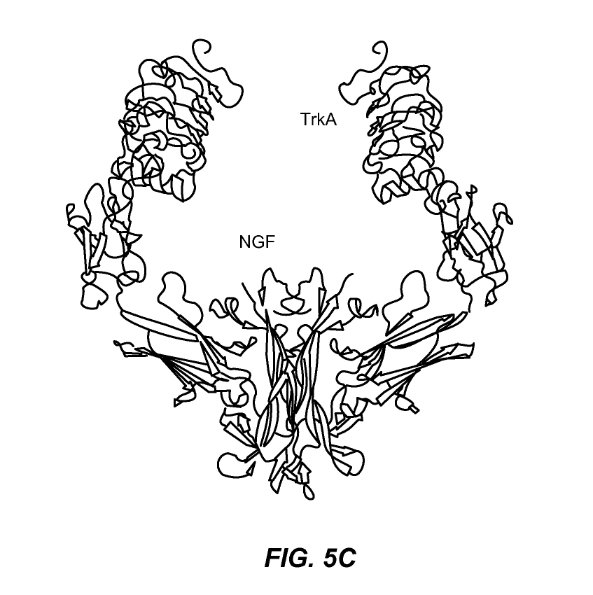

Several crystal structures for receptor-cysteine-knot growth factor ligand complexes, such as nerve growth factor (NGF) (28-30), vascular endothelial growth factor (VEGF) (31) two glial cell-derived neurotrophic factor (GDNF) family members (32), and others; these structures can serve as instructive comparisons with the mode of ligand engagement mediated by IL-17RA (FIG. 5). In the complex of NGF bound to the p75 neurotrophin receptor (p75NTR, a death receptor family member) (28, 30), the receptor bears no structural similarity to IL-17RA; however, like IL-17RA, p75NTR engages NGF within a concave groove at the ligand dimer interface (FIG. 5B). In the TrkA complex with NGF (29, 33), an immunoglobulin (Ig)-domain in TrkA, which is structurally related to the FnIII domains of IL-17RA, is used for ligand binding. However, the Ig-domain of TrkA binds end-on to a flat face in the ‘saddle’ of NGF formed by the NGF β-sheets; thus the mode of binding is distinct (FIG. 5C). Interestingly, the NGF-p75NTR complex has been reported as both 1:2 and 2:2 complexes that may represent partial and complete forms of a homodimeric p75 signaling complex, respectively (28, 30). However, in that case, homodimeric NGF ligand engages two identical p75 molecules, and thus does not require a structural mechanism for the symmetric dimeric ligand to heterodimerize two different receptors.

### Example 7

### Human IL-17RC or Human IL-17RA Binding

Binding of Biotinylated Cytokines to Transfected Cells.

Baby Hamster Kidney (BHK) cells transfected with expression vectors encoding human IL-17RA, human IL-17RC, or both of these receptors are assessed for their ability to bind biotinylated human IL-17A, human IL-17F, and their variants including antagonists described herein. Cells are harvested with versene, counted and diluted to 10⁷ cells per ml in staining media (SM), which is HBSS plus 1 mg/ml bovine serum albumin (BSA), 10 mM HEPES, and 0.1% sodium azide (w/v). Biotinylated human IL-17A, human IL-17F, and other proteins of interest are incubated with the cells on ice for 30 minutes at various concentrations. After 30 minutes, excess protein is washed away with SM and the cells are incubated with a 1:100 dilution of streptavidin conjugated to phycoerythrin (SA-PE) for 30 minutes on ice. Excess SA-PE is washed away and cells are analyzed by flow cytometry. The amount of binding is quantitated from the mean fluorescence intensity of the staining.

Binding of Biotinylated Cytokines to Human Peripheral Blood Mononuclear Cells (PBMC).

PBMCs are prepared from whole blood by Ficoll density gradient centrifugation. PBMC at 10⁷ cells per ml are simultaneously incubated with biotinylated IL-17A or IL-17F or proteins of interest at 1 μg/ml and fluorochrome conjugated antibodies to specific cell surface proteins that are designed to distinguish various white blood cell lineages. These markers include CD4, CD8, CD19, CD11b, CD56 and CD16. Excess antibody and cytokine are washed away, and specific cytokine binding is detected by incubating with SA-PE as described above. Samples are analyzed by flow cytometry.

Inhibition of Specific Binding.

Binding studies are performed as discussed above, but excess unlabeled human IL-17A and IL-17F or excess unlabeled proteins of interest such as proteins described herein are included in the binding reaction. In studies with BHK cells, the amount of unlabeled protein is varied over a range of concentrations and unlabeled IL-17A and proteins of interest are evaluated for ability to compete for binding of both IL-17A and IL-17F to both IL-17RC and IL-17RA.

### Example 8

### Murine NIH3T3 Cells Respond to Human IL-17A and IL-17F

Murine NIH3T3 cells are transfected with Kz142 adenovirus particles containing a consensus NF-κB binding site, the tandem NF-κB binding sites of the human immunodeficiency virus-1 long terminal repeat, two copies of the collagenase AP-1 element, and a single copy of the c-Jun TRE ligated into a luciferase reporter cassette and placed in the pACCMV.pLpA adenoviral shuttle vector as described in Blumberg et al. (2001) Cell 104:9-19.

Following the overnight incubation with the adenovirus particle reporter, treatments (e.g., with IL-17A, IL-17F, or others proteins of interest) are prepared in serum free media containing 0.28% BSA. The adenovirus particles and media are removed and the appropriate doses are given. Incubation at 37° C. and 5% CO₂ is continued for 4 hours, after which the media is removed, cells lysed for 15 minutes and mean fluorescence intensity (MFI) measured using the luciferase assay system and reagents. (Cat#e1531 Promega, Madison, Wis.) and a microplate luminometer. Stable cell lines can also be made. Stable and/or transient cell lines can be used to evaluate a protein described herein for activity.

### Example 9

### IL-17A Induces Elevated Levels of IFNγ and TNFα in Human Peripheral Blood Mononuclear Cells

Human peripheral blood mononuclear cells (PBMC) are purified by Ficoll density gradient centrifugation and then incubated overnight at 37° C. in media alone, 50 ng/ml anti-human CD3 antibody, or the combination of 50 ng/ml anti-human CD3 antibody plus 1 μg/ml anti-human CD28 antibody. Replicate cultures for each of these conditions are set up and are given no cytokine, 25 ng/ml human IL-17A, 25 ng/ml human IL-17F, or varying concentrations of a protein of interest (for example in the presence of cytokine). After 24-hour incubations, supernatants from each culture are harvested and assayed for cytokine content using B-D Bioscience's human Th1/Th2 Cytometric Bead Array (CBA). We expect cultures stimulated with either anti-CD3 or anti-CD3 plus anti-CD28 and supplemented with IL-17A to contain significantly elevated levels of IFNγ and TNFα over cultures with no cytokine added or those that received IL-17F. Proteins of interest can be evaluated for their ability to inhibit IL-17A induction of IFNγ and TNFα.

### Example 10

### Mouse Collagen Induced Arthritis (CIA) Model

The mouse Collagen Induced Arthritis (CIA) model can be used to evaluate therapeutic potential of drugs (such as proteins described herein) to treat human arthritis. Eight to ten-week old male DBA/IJ mice (Jackson Labs; 25-30 g each) are used for these studies. On day-21, animals are given an intradermal tail injection of 100 μL of 1 mg/ml chick type II collagen formulated in Complete Freund's Adjuvant and three weeks later on Day 0 mice are given the same injection except prepared in Incomplete Freund's Adjuvant. Animals begin to show symptoms of arthritis following the second collagen injection, with most animals developing inflammation within 1-2 weeks. The extent of disease is evaluated in each paw by using a caliper to measure paw thickness, and by assigning a clinical score (0-3) to each paw: 0=Normal, 0.5=Toe(s) inflamed, 1=Mild paw inflammation, 2=Moderate paw inflammation, and 3=Severe paw inflammation as detailed below.

Incidence of disease in this model is typically 95-100%, and 0-2 non-responders (determined after 6 weeks of observation) are typically seen in a study using 40 animals. Note that as inflammation begins, a common transient occurrence of variable low-grade paw or toe inflammation can occur. For this reason, an animal is not considered to have established disease until marked, persistent paw swelling has developed.

All animals are observed daily to assess the status of the disease in their paws, which is done by assigning a qualitative clinical score to each of the paws. Every day, each animal has its four paws scored according to its state of clinical disease. To determine the clinical score, the paw is thought of as having three zones, the toes, the paw itself (manus or pes), and the wrist or ankle joint. The extent and severity of the inflammation relative to these zones is noted including: observation of each toe for swelling; torn nails or redness of toes; notation of any evidence of edema or redness in any of the paws; notation of any loss of fine anatomic demarcation of tendons or bones; evaluation of the wrist or ankle for any edema or redness; and notation if the inflammation extends proximally up the leg. A paw score of 1, 2, or 3 is based first on the overall impression of severity, and second on how many zones are involved.

Treatments: Established disease is defined as a qualitative score of paw inflammation ranking 1 or more. Once established disease is present, the date is recorded, designated as that animal's first day with “established disease”, and treatment started. Mice are treated with PBS, or with varying doses of the protein of interest, i.p. every other day for a total of five doses: 150 μg; 75 μg; 25 μg; and 10 μg.

Blood is collected throughout the experimental period to monitor serum levels of anti-collagen antibodies, as well as serum immunoglobulin and cytokine levels. Animals are euthanized 48 hours following their last (5th) treatment, about 10 days following disease onset. Blood is collected for serum, and all paws are collected into 10% NBF for histology. Serum is collected and frozen at −80° C. for immunoglobulin and cytokine assays. A dose-dependent, significant reduction in clinical score severity in treated mice indicates a biological effect for the protein in this test system.

### Example 11

### Additional Disease Model

The Inflammatory Bowel Disease (IBD) model is designed to show that cultured intestinal tissue from patients with IBD produce higher levels of inflammatory mediators compared to tissue from healthy controls. This enhanced production of inflammatory mediators (including but not limited to IL-1β, IL-4, IL-5, IL-6, IL-8, IL-12, IL-13, IL-15, IL-17 A and F, IL-18, IL-23, TNF-α, IFN-γ, MIP family members, MCP-1, G- and GM-CSF, etc.) contributes to the symptoms and pathology associated with IBDs such as Crohn's disease (CD) and ulcerative colitis (UC) by way of their effect(s) on activating inflammatory pathways and downstream effector cells. These pathways and components then lead to tissue and cell damage/destruction observed in vivo. Therefore, this model can simulate this enhanced inflammatory mediator aspect of IBD. Furthermore, when intestinal tissue from healthy controls or from human intestinal epithelial cell (IEC) lines is cultured in the presence of these inflammatory components, inflammatory pathway signaling can be observed, as well as evidence of tissue and cell damage.

Therapeutics that would be efficacious in human IBD in vivo would work in the above ex vivo or IEC models by inhibiting and/or neutralizing the production and/or presence of inflammatory mediators.

In this model, human intestinal tissue is collected from patients with IBD or from healthy controls undergoing intestinal biopsy, re-sectioning or from post-mortem tissue collection, and processed using a modification of Alexakis et al. (Gut 53:85-90; 2004). Under aseptic conditions, samples are gently cleaned with copious amounts of PBS, followed by culturing of minced sections of tissue, in the presence of complete tissue culture media (plus antibiotics to prevent bacterial overgrowth). Samples from the same pool of minced tissue are treated with one of the following: vehicle (PBS); recombinant human (rh) IL-17A; rhIL-17F; or rhIL-17A+rhIL-17F. In addition, these samples can be treated with or without an antagonist of either IL-17A, IL-17F, IL-17B, IL-17C, IL-17D, and IL-17E alone or in combination. This experimental protocol is followed for studies with human IEC lines, with the exception that cells are passaged from existing stocks. After varying times in culture (from 1 h to several days), supernatants are collected and analyzed for levels of inflammatory mediators, including those listed above. In samples from patients with IBD or in samples treated with rhIL-17A and/or F, levels of inflammatory cytokines and chemokines are elevated compared to untreated healthy control tissue samples. Proteins of interest can be evaluated for ability to reduce the production of inflammatory mediators, and thus, to be efficacious in human IBD.

Proteins of interest can be evaluated in a mouse model for dry eye disease. Dry eye can be induced in mice by subcutaneous injection of scopolamine and then placement of the mice in controlled-environment chambers. The controlled environment chamber can be controlled for relative humidity, temperature, and air flow. See, e.g., Barabino et al., Invest. Ophth. Vis. Sci., 46:2766-71, 2005. Various mouse strains can be used. These include, e.g., C57BL/6, BALB/c, NZB/W, and MLR/lpr, MLR/+. Other animals, e.g., rabbits, rats, monkeys, dogs, and cats, can also be used as dry eye disease models. See e.g., Nguyen and Peck, Ocul. Surf., 7(1):11-27, 2009 (including Table 1), and Barabino and Dana, Invest. Ophth. Vis. Sci., 45(6): 1641-46, 2004.

By way of example, dry eye can be induced in normal healthy 6 to 10 weeks old female C57BL/6 mice by continuous exposure to dry environment in a controlled environmental chamber with humidity less than 30% (generally about 19%), high airflow (generally greater than about 15 liters/minute) and constant temperature (about 22° C.). The mice placed in the chamber are also treated with scopolamine to inhibit tear secretion. One quarter of a sustained release transdermal scopolamine patch (Novartis, Summit N.J.) is applied to the depilated mid-tail of mice every 48 hours, or the scopolamine can be injected, e.g., 750 μg, twice daily subcutaneously. The combination of the controlled environmental chamber and scopolamine produces severe dry eye in a relative short timeframe (about 2-4 days). Mice can be treated after disease onset with a protein of interest for 7 to 14 days under these conditions and compared to placebo or vehicle treated controls. Mice can be monitored and evaluated for dry eye, e.g., by performing: (a) an assessment of aqueous tear production; (b) corneal fluorescein staining which is a marker of corneal surface damage; (c) an assessment of goblet cell density in the superior and inferior conjunctiva; (d) general ophthalmic examination, e.g., for conjunctival epithelial morphology; (e) scanning electron microscope examination of corneal surface; and (f) immunohistochemistry.

### Example 12

### Rheumatoid Arthritis (RA) and Osteoarthritis (OA) Model

This model is designed to show that human synovial cultures (including synovial macrophages, synovial fibroblasts, and articular chondrocytes) and explants from patients with RA and OA produce higher levels of inflammatory mediators compared to cultures/explants from healthy controls. This enhanced production of inflammatory mediators (including but not limited to oncostatin M, IL-1β, IL-6, IL-8, IL-12, IL-15, IL-17 A and F, IL-18, IL-23, TNF-α, IFN-γ, IP-10, RANTES, RANKL, MIP family members, MCP-1, G- and GM-CSF, nitric oxide, etc.) contributes to the symptoms and pathology associated with RA and OA by way of their effect(s) on activating inflammatory pathways and downstream effector cells. These pathways and components then lead to inflammatory infiltrates, cartilage and matrix loss/destruction, bone loss, and upregulation of prostaglandins and cyclooxygenases. Therefore, this model can simulate the destructive inflammatory aspects of RA and OA in in vitro and ex vivo experiments. Furthermore, when explants and synovial cultures from healthy controls are cultured in the presence of several of these inflammatory components (e.g. oncostatin M, TNF-α, IL-1β, IL-6, IL-17A and F, IL-15, etc.), inflammatory pathway signaling can be observed. Therapeutics that would be efficacious in human RA in vivo may have an effect in the above in vitro and ex vivo models by inhibiting and/or neutralizing the production and/or presence of inflammatory mediators.

In this model, human synovial explants are collected from patients with RA, OA, or from healthy controls undergoing joint replacement or from post-mortem tissue collection, and processed using a modification of Wooley and Tetlow (Arthritis Res 2: 65-70, 2000) and van't H of et al. (Rheumatology 39:1004-1008, 2000). Cultures of synovial fibroblasts, synovial macrophages and articular chondrocytes are also studied. Replicate samples are treated with one of the following: vehicle (PBS); recombinant human (rh) IL-17A; rhIL-17F; or rhIL-17A+rhIL-17F, and some samples contain various combinations of oncostatin M, TNF-α, IL-1, IL-6, IL-17A, IL-17F, and IL-15. In addition, these can be evaluated in the presence or absence of a protein of interest. After varying time of culture (from 1 h to several days), supernatants are collected and analyzed for levels of inflammatory mediators, including those listed above. In samples from patients with RA or OA, or in samples treated with rhIL-17A and/or F (either alone or in combination with other inflammatory cytokines), levels of inflammatory cytokines and chemokines are elevated compared to untreated healthy control explants or in untreated cell cultures. Proteins of interest can be evaluated for ability to reduce the production of inflammatory mediators, and thus, to be efficacious in human RA and OA.

### Example 13

### Induction of G-CSF, IL-6 and IL-8

Human small airway epithelial cells (SAEC) treated with human IL-17A or with human IL-17F can show a dose-dependent induction of G-CSF, IL-6, and IL-8, e.g., by evaluation of cell supernatants 48 hr after treatment. Proteins of interest can be evaluated for their ability to inhibit this induction.

### Example 14

### Human Rheumatoid Arthritis (“RA”) and Osteoarthritis (“OA”) Samples

These models are designed to show that human synovial cultures (including synovial macrophages, synovial fibroblasts, and articular chondrocytes) and explants from patients with RA and OA produce higher levels of inflammatory mediators compared to cultures/explants from healthy controls, which in turn can contribute to the degradation of extracellular matrix components (e.g. bone, cartilage, etc), which is a hallmark of these diseases. In addition, the co-culture models described below are designed to show that inflammatory mediators present in RA/OA synovial fluid and/or activated T cells can also result in greater inflammation and matrix degradation.

The enhanced production of inflammatory mediators (including but not limited to oncostatin M, IL-1β, IL-6, IL-8, IL-12, IL-15, IL-17 A and F, IL-18, IL-23, TNF-α, IFN-γ, IP-10, RANTES, RANKL, MIP family members, MCP-1, MMP-9, G- and GM-CSF, nitric oxide, etc.) contributes to the symptoms and pathology associated with RA and OA by way of their effect(s) on activating inflammatory pathways and downstream effector cells. These pathways and components then lead to inflammatory infiltrates, cartilage and matrix loss/destruction, bone loss, and upregulation of matrix metalloproteases, prostaglandins and cyclooxygenases. Therefore, these models can simulate the destructive inflammatory aspects of RA and OA in in vitro and ex vivo experiments. Furthermore, when explants and synovial cultures from healthy controls are cultured in the presence of exogenously added inflammatory components (e.g. oncostatin M, TNF-α, IL-1β, IL-6, IL-17A and F, IL-15, etc.), or alternatively, in the presence of synovial fluid from RA patients (which would contain inflammatory components endogenously), inflammatory and degradative pathway signaling can be observed. Therapeutics that would be efficacious in human RA in vivo would work in the above in vitro and ex vivo models by inhibiting and/or neutralizing the production and/or presence of inflammatory mediators.

In these models, human synovial explants are collected from patients with RA, OA, or from healthy controls undergoing joint replacement or from post-mortem tissue collection, and processed using a modification of Wooley and Tetlow (Arthritis Res 2: 65-70; 2000) and van't H of et al. (Rheumatotogy 39:1004-1008; 2000). Cultures of synovial fibroblasts, synovial macrophages and articular chondrocytes are also studied. Replicate samples are treated with one of the following: vehicle (PBS); recombinant human (rh) IL-17A; rhIL-17F; or rhIL-17A+rhIL-17F, and some samples contain various combinations of oncostatin M, TNF-α, IL-1, IL-6, IL-17A, IL-17F, and IL-15. A separate set of samples is treated with activated human T cells, or synovial fluid from healthy controls or patients with RA or OA. After varying time of culture (from 1 h to several days), supernatants and cells are collected and analyzed for levels of inflammatory mediators and cartilage/bone/matrix biomarkers, including those listed above. Samples can be treated with a protein of interest and evaluated for ability to reduce the production of inflammatory and cartilage/bone/matrix degradative mediators, and thus, to be efficacious in human RA and OA.

### Example 15

### Single Chain Human IL17A:IL17F Heterodimers

Recombinant human IL17A:IL17F heterodimer protein or recombinant IL17A:IL17F-variant is produced from expression of the appropriate single chain construct in CHO DXB11 cells and cell culture in a WAVE apparatus. One construct is comprised of sequences for human IL-17A at the N-terminus with IL-17F at the C-terminus linked with a (G₄S)₃ linker; another exemplary construct is comprised of sequences for human IL-17A at the N-terminus with a IL-17F-variant at the C-terminus linked with a (G₄S)₃ linker. A His tag can be included at the C-terminus for product capture. An exemplary purification method is described in US 20080241138. Briefly, it can include an acid precipitation step, filtration, followed by chromatography. For example, approximately 10 L of conditioned media are harvested and sterile filtered using a 0.2 μm filter. The media is adjusted to pH 5.0 with addition of acetic acid while stirring. After precipitation, the pH-adjusted media is again filtered through a two stage 0.8 to 0.2 micron filter. The media can then be subjected to cation exchange chromatography on SP Fast Flow resin, and eluted with a salt gradient. Peak fractions can then be subjected to IMAC chromatography, e.g., using a 5 mL HISTRAP® IMAC column (GE Healthcare). After elution with imidazole, peak fractions can be subjected size exclusion chromatography, e.g., on SUPERDEX® 200. Peak fractions can then be pooled and used. Fractions can be evaluated by Western analysis (e.g., with an anti-His tag antibody) and/or by SDS-PAGE with Coomassie gel staining.

### Example 16

### Expression and Purification of IL-17RA and IL-17F

The native signal peptide and extracellular region of human IL-17RA (residues 1-286) was cloned into the BACMAM® expression vector pVLAD637.

Recombinant protein was transiently expressed in suspension 293 GnTI-cells grown in PRO293™ media (Lonza) supplemented with 1% fetal calf serum (FBS) and 10 mM Na butyrate at 37° C. Full length IL-17F with a C-terminal hexa-His tag was cloned into the pAcGP67-A expression vector (BD Biosciences) and the protein secreted by High Five insect cells grown in INSECT XPRESS™ media (Lonza) at 27° C. The supernatants containing the IL-17RA and IL-17F proteins were mixed and concentrated before Ni-affinity purification. The IL-17RA protein was deglycosylated via endoglycosidase-H treatment and the IL-17RA and IL-17F purification tags cleaved using 3C-protease and carboxypeptidase A (Sigma-Aldrich). The protein complex was subjected to reductive lysine methylation using dimethylamine-borane complex and formaldehyde as described by Walter et al. (38). The IL-17RA-IL-17F complex was further purified using a SUPERDEX® 200 size exclusion column (GE Healthcare) equilibrated in 10 mM HEPES pH 7.4 and 150 mM NaCl. Fractions containing the IL-17RA-IL-17F complex were concentrated to ˜15 mg/ml for crystallization trials.

Seleno-methionine (SeMet) labeled IL-17RA protein was prepared as described with the following modifications (39). Untransfected adherent 293 GnTI-cells were cultivated in FBS-supplemented DMEM media (Invitrogen). The media was exchanged after a single phosphate-buffered saline wash, for Met and Cys-free DMEM (Invitrogen) supplemented with 40 mg/l L-Cys, 45 mg/l selenon-L-Met, 2% FBS, L-glutamate, Na pyruvate, IL-17RA BacMam virus and 10 mM Na butyrate. Expression was allowed to proceed for 72 hours. IL-17RA-SeMet protein supernatants were mixed with IL-17F and purified as described above.

For binding experiments, proteins were expressed and purified essentially as described above. The IL-17RA, IL-17RB and IL-17RC extracellular domains were expressed by 293s GnTI-cells with and without a C-terminal BirA ligase tag. IL-17RC was expressed with an additional C-terminal Fc tag that was cleaved by 3C-protease prior to size exclusion chromatography. IL-17A, IL-17F and IL-25 cytokines were expressed by High Five cells with C-terminal hexa-His tags. Proteins were enzymatically biotinylated using BirA ligase and purified via size exclusion chromatography.

### Example 17

### Crystallization and X-Ray Data Collection

IL-17RA-IL-17F complexes were initially grown via hanging-drop vapor diffusion in 10% PEG6000 and 0.1 M bicine pH 9.0. Optimized native and SeMet protein complex crystals were grown in PEG6000 (4-14%) and 0.1 M CAPSO buffer (pH 9.1-9.3) with 20 mM CaCl₂ or 10 mM CaCl₂ and 1.5% w/v trimethylamine N-oxide dihydrate added directly to the protein-precipitant drop. Heavy metal derivatives were prepared by soaking the crystals in well solution supplemented with 0.5 mM K₂PtCl₄ and 2% ethylene glycol for 6 hours. Crystals were cryo-protected prior to data collection in the well solution plus 20-25% ethylene glycol and cooled to 100 K. The crystals belong to the space group P41212 and have unit cells dimensions of ˜171, 171, 83 Å. The initial native data set was collected at Stanford Synchrotron Radiation Lightsource (SSRL) beamline 9-2 (Stanford, Calif.). The Pt-derivative and SeMet datasets were collected at SSRL beamline 11-1. The higher resolution native dataset was collected at the Advanced Photon Source (APS) beamline ID-23D (Argonne, Ill.). All data was indexed and integrated using the program Mosflm40 and scaled with SCALA from the CCP4 suite (41). The diffraction is anisotropic and the initial native dataset was also subjected to ellipsoidal truncation and anisotropic scaling using the diffraction anisotropy server (42) rendering a data set scaled to 3.4, 3.4 and 3.9 Å.

### Example 18

### Structure Determination and Refinement

A molecular replacement solution for a single IL-17F homodimer was determined using the program Phaser43 with the previously determined 2.85 Å IL-17F structure as a model (PDB ID 1JPY) (7). The initial maps showed additional density on one side of the IL-17F dimer illuminating the binding site for IL-17RA. Phases were calculated using a K₂PtCl₄ derivative via single isomorphous replacement with anomalous scattering (SIRAS) in the program Sharp (44). Density modified maps were calculated assuming 71% solvent and including the partial model from the IL-17F molecular replacement for 10 out of 20 rounds. A partial model of the IL-17RA main chain was manually built into this map using the program Coot (45).

The position of the IL-17RA Met residues was calculated via fast Fourier transform (FFT) to generate an anomalous difference map using the program FFT in the CCP4 suite. As the SeMet dataset was not isomorphous with the native dataset and the signal too weak to locate the sites via single anomalous difference (SAD) phasing methods, the partially built model was used as a molecular replacement model for the SeMet dataset and the calculated phases used to find the selenonium peaks. Three of a potential six SeMet residues were located, corresponding to IL-17RA Met159, Met166 and Met218. These Met positions, in addition to the predicted Asn-linked glycosylation sites and disulfide bonds were used to register the polypeptide in the density and complete building the initial IL-17RA model. Iterative rounds of coordinate and B-factor refinement were performed using the program Phenix46 intersected with manual model building in Coot. Initial rounds of model building utilized B-factor sharpened σ_{A}-weighted phased-combined maps calculated by the program CNS (47). The final model was refined to 3.3 Å with an R_{factor} and R_{free} of 22.7% and 25.3% respectively. There is one IL-17RA-IL-17F complex in the asymmetric unit. The model includes a dimethyl-lysine at position 43 of the IL-17RA chain, five single N-Acetylglucosamine (GlcNAc) sites on the IL-17RA chain, one site with two GlcNAc residues on the IL-17F chain B and a calcium ion. The programs PROCHECK48 and WHAT_CHECK (49) were used to assess the geometry of the final model. The CCP4 suite programs Contact and Areaimol were used to determined the interface contacts and buried surface area respectively. All structural figures were generated using the program Pymol (50).

### Example 19

### Affinity Measurements

Binding affinities were calculated via surface plasmon resonance (SPR) on a BIACORE® T100 (GE Healthcare). C-terminally biotinylated IL-17 receptors were coupled to immobilized streptavidin on either an SA or CM4-sensor chip (GE Healthcare). An irrelevant, biotinylated protein was captured at equivalent immobilization densities to control flow cells. To measure the second receptor binding interaction, the cytokine was first captured to the immobilized receptor, followed by the second receptor injection. Low coupling densities (200-400 RU) and excess cytokine concentrations were used to optimize the number of cytokine homodimers bound to a single receptor. The surface was regenerated using 3 M MgCl₂ between each cycle. For kinetic experiments, a flow rate of 50 μl/min was used. Data was analyzed using BIACORE® T100 evaluation software Version 2.0 (GE Healthcare). Affinities are reported as the mean of at least two independent experiments±the standard error of the mean (s.e.m.).

### Example 20

The structure of IL-17F bound to IL-17RA was analyzed.

Asn89 is conserved between IL-17A and IL-17F and in chain A forms a hydrogen bond to the IL-17RA backbone in the site 3 pocket. A substitution, e.g., with alanine, would remove the interaction.

Gln-95 in chain A makes some hydrophobic interactions in the site 2 pocket. Substitution with a small residue such as alanine can be used to disrupt interactions and substitution with a bulky group, e.g. tryptophan, can be used to block the IL-17RA loop insertion.

Arg37 in chain B of IL-17F (SEQ ID NO:12): Arg37 forms potential hydrogen bonds and salt bridges with IL-17RA, and position 41 is serine although the side chain could not be modeled with confidence. Alanine at Arg37 would disrupt the hydrogen bonds and salt bridges. IL-17A does not have a charged residue at position corresponding to Arg37 however has a lysine at position 37 in SEQ ID NO:2 (corresponding to position 39 in SEQ ID NO:12). Substituting the charged residue at position 37 in IL-17F (SEQ ID NO:12) or Lys-37 or Arg 38 in IL-17A (SEQ ID NO:2) for an alanine residue can be used to reduce affinity for the receptor. Substitution of these positions with a residue with the opposite charge, e.g., a glutamic acid or aspartic acid, can also be used.

Arg42, Arg47, and Arg102 in chain B are conserved arginine residues. In the IL-17RA-IL-17F complex these arginine residues may form hydrogen bonds and salt bridges in site 3 (Arg42 and Arg47) and site 1 (Arg102). As Arg42 and Arg47 are in a similar environment, they can both be targeted together. Any one, two or all three can be substituted, e.g., in the same molecule, e.g., with an uncharged residue or an acidic residue. In addition, in the context of IL-17A, Arg38 of SEQ ID NO:2 can be substituted, e.g., in combination with positions corresponding to Arg47 and Arg102.

Tyr63 in chain B is conserved in four of the six IL-17 cytokines, and is hydrophobic in the other two. Tyr63 makes extensive hydrophobic interactions, including with Trp31 that is buried in the centre of site 1. Tyr63 also forms potential hydrogen bonds with other site 1 residues. An alanine substitution can be used to disrupt the interactions and substitution with a charged residue (e.g., lysine) can be used to block the pocket.

Val68 in chain B (Trp in IL-17A) forms hydrophobic interactions with the receptor at site 2. Substituting Val68 for example with a long, polar side chain (e.g. glutamine) can be used to disrupt loop insertion.

Phe111 in chain B forms hydrophobic interactions at the top of the site 1 pocket. Substitution with an alanine and/or in combination with Arg102 substitution can be used to disrupt these interactions at the binding interface.

In addition the observations in Table 3 were made (residues identified with reference to IL-17F and SEQ ID NO:12).

TABLE 3

| | | Column 3 | |
|---|---|---|---|
| | | Buried on | Column 4 |
| Column 1 | Column 2 | IL-17RA | Distance to |
| Chain:Residue | Site | binding | IL-17RA |
| A:MET25 | 1 | 0.3 | 3.33 |
| B:ILE29 | 1 | 0.3 | 3.95 |
| B:ILE31 | 1 | 0.14 | 3.08 |
| B:TRP58 | 1 | 0.22 | 3.39 |
| B:ASN61 | 1 | 0.08 | 4.76 |
| B:TYR63 | 1 | 0.49 | 3.11 |
| B:PRO64 | 1 | 0.28 | 3.16 |
| B:SER65 | 1 | 0.22 | 4.23 |
| B:VAL100 | 1 | 0.08 | 4.26 |
| B:ARG102 | 1 | 0.45 | 2.72 |
| B:HIS104 | 1 | 0.32 | 3.27 |
| B:VAL109 | 1 | 0.26 | 4.53 |
| B:PHE111 | 1 | 0.14 | 2.84 |
| A:ILE93 | 2 | 0.02 | 5.24 |
| A:GLN94 | 2 | 0.05 | 4.39 |
| A:GLN95 | 2 | 0.25 | 2.8 |
| A:GLU96 | 2 | 0.46 | 3.49 |
| A:LEU117 | 2 | 0.06 | 5.14 |
| B:GLN36 | 2 | 0.29 | 3.74 |
| B:ARG37 | 2 | 0.41 | 2.47 |
| B:SER41 | 2 | 0.72 | 3.15 |
| B:ASN43 | 2 | 0.46 | 2.92 |
| B:GLU45 | 2 | 0.14 | 3.36 |
| B:TYR54 | 2 | 0.23 | 3.54 |
| B:VAL56 | 2 | 0.41 | 3.59 |
| B:VAL68 | 2 | 0.42 | 3.34 |
| A:LEU75 | 3 | 0.34 | 4.02 |
| A:ILE86 | 3 | 0.45 | 3.97 |
| A:SER87 | 3 | 0.02 | 4.56 |
| A:ASN89 | 3 | 0.46 | 2.97 |
| A:VAL91 | 3 | 0.08 | 4.5 |
| A:VAL125 | 3 | 0 | 4.22 |
| A:PRO127 | 3 | 0.2 | 3.23 |
| A:VAL128 | 3 | 0.19 | 3.41 |
| A:ILE129 | 3 | 0.23 | 4 |
| A:HIS130 | 3 | 0.43 | 3.21 |
| A:HIS131 | 3 | 0.3 | 3.08 |
| A:VAL132 | 3 | 0.42 | 3.56 |
| B:ARG42 | 3 | 0.27 | 2.91 |
| B:ILE44 | 3 | 0.01 | 4.42 |
| B:ARG47 | 3 | 0.07 | 2.75 |
| A:LYS115 | 1 and 2 | 0.22 | 4.15 |
| B:GLU66 | 1 and 2 | 0.56 | 3.57 |
| B:MET40 | 2 and 3 | 0.55 | 3.74 |

Column 3 provides the fraction of side chain solvent accessible surface area (SASA) buried by binding to IL-17RA, normalized by SASA in the unfolded state. Column 4 provides the minimum distance from any side chain atom in the residue to any atom in IL-17RA (in Angstroms).

### Example 21

### IL-17 Heterodimers Formed By Acid-Base Zippers

Several mutated IL-17 cytokine dimer proteins were designed as heterodimers of two different subunit sequences. One approach to preparing such heterodimers is by fusion of each respective subunit to one of two heterodimeric zipper sequences, e.g., one of a pair acidic-basic zippers. See, e.g., O'Shea et al., Curr Biol. (1993), 3(10):658-67. In this example, one subunit of IL-17A was expressed with a C-terminal tag that contained an acidic sequence and a hexahistidine tag. Another subunit of IL-17A was expressed with a C-terminal tag that contained a basic sequence and a hexahistidine tag. The sequence of these subunits is as follows:

IL-17A-Acid zipper:
(SEQ ID NO: 17)
GITIPRNPGCPNSEDKNFPRTVMVNLNIHNRNTNTNPKRSSDYYNR
STSPWNLHRNEDPERYPSVIWEAKCRHLGCINADGNVDYHMNSVPI
QQEILVLRREPPHCPNSFRLEKILVSVGCTCVTPIVHHVASGGGGS
RGGLEVLFQGPEFGGSTTAPSAQLEKELQALEKENAQLEWELQALE
KELAQHHHHHH
IL-17A-Base zipper:
(SEQ ID NO: 18)
GITIPRNPGCPNSEDKNFPRTVMVNLNIHNRNTNTNPKRSSDYYNR
STSPWNLHRNEDPERYPSVIWEAKCRHLGCINADGNVDYHMNSVPI
QQEILVLRREPPHCPNSFRLEKILVSVGCTCVTPIVHHVASGGGGS
RGGLEVLFQGPEFGGSTTAPSAQLKKKLQALKKKNAQLKWKLQALK
KKLAQHHHHHH

The constructs were co-transfected into 293 cells and protein was recovered.

### Example 22

### IL-17 Heterodimers Formed By Single Chain Fusion

Another approach to preparing heterodimers is by covalently linking the two subunits using a flexible peptide linker and expressing them as a single polypeptide chain. An example of a single chain IL-17A molecule is as follows:

(SEQ ID NO: 19)
GITIPRNPGCPNSEDKNFPRTVMVNLNIHNRNTNTNPKRSSDYYNR
STSPWNLHRNEDPERYPSVIWEAKCRHLGCINADGNVDYHMNSVPI
QQEILVLRREPPHCPNSFRLEKILVSVGCTCVTPIVHHVASGGGGS
GGGGSGGGGSGGGGSGGGGSGGGGSGITIPRNPGCPNSEDKNFPRT
VMVNLNIHNRNTNTNPKRSSDYYNRSTSPWNLHRNEDPERYPSVIW
EAKCRHLGCINADGNVDYHMNSVPIQQEILVLRREPPHCPNSFRLE
KILVSVGCTCVTPIVHHVASHHHHHH

This protein was expressed in 293 cells. Supernatants from the cells were run on non-reducing gels and Western blot analysis using an anti-hexahistidine antibody was performed. A substantial portion of the His-tagged protein migrated at a molecular weight (˜35 kDa) corresponding to the monomeric form of the single-chain protein.

### Example 23

### Assay for IL-17 Activity

Control IL-17A and IL-17F proteins and mutant IL-17A and IL-17F proteins were evaluated in a cell-based functional assay according to the method of Fossiez et al., J. Exp. Med. 183(6):2593-603 (1996). Briefly, MRC-5 human embryonic fibroblast cells were subcultured in 96-well plates at a concentration of 1×10⁵ cells/well in DMEM with 10% FBS. Control proteins and proteins of interest in PBS, pH 7.4, were added to respective wells at a final concentration of 0.1-10,000 ng/mL. Cells were incubated an additional 48 hours. IL-6 concentration in the supernatants was then measured by ELISA using the Thermo Scientific Human IL-6 Screening Set (cat#ENESS0005). Using this assay, IL-17A and IL-17F control proteins were observed to have an EC50 within published ranges of 1-10 ng/mL for IL-17A and 50-100 ng/mL for IL-17F.

### Example 24

### Single Mutations in IL-17A and IL-17F

Single mutations were made in both subunits of the IL-17A/IL-17A dimer and the IL-17F/IL-17F dimer—that is proteins were produced having two identical subunits, each containing a single mutation. Tables 4 and 5 list the reduction in activity observed for each mutation in this format using the assay described above in Example 23:

TABLE 4

| | Mutated | Mutated | Mutated | | % |
|---|---|---|---|---|---|
| | Position in | Position in | Position in | | Activity |
| | SEQ ID | SEQ ID | SEQ ID | | of |
| Mutation | NO: 2 | NO: 20 | NO: 12 | Protein | Wildtype |
| K→E | 37 | 38 | 39 | A/A | 90.3 |
| R→E | 38 | 39 | 40 | A/A | 92.8 |
| R→A | 45 | 46 | 47 | A/A | 37.3 |
| Y→A | 61 | 62 | 63 | A/A | 94.2 |
| Y→K | 61 | 62 | 63 | A/A | 73.0 |
| W→Q | 66 | 67 | 68 | A/A | 94.3 |
| N→A | 87 | 88 | 89 | A/A | 90.9 |
| N→W | 87 | 88 | 89 | A/A | 102.7 |
| Q→A | 93 | 94 | 95 | A/A | 99.1 |
| Q→W | 93 | 94 | 95 | A/A | 32.6 |
| R→A | 100 | 101 | 102 | A/A | 62.6 |
| F→Q | 109 | 110 | 111 | A/A | 82.8 |

TABLE 5

| | Mutated Position in | | % Activity |
|---|---|---|---|
| Mutation* | SEQ ID NO: 12 | Protein | of Wildtype |
| R→A | 37 | F/F | 75.4 |
| R→E | 37 | F/F | 67.3 |
| R→A | 42 | F/F | 47.0 |
| R→E | 42 | F/F | 10.9 |
| R→A | 47 | F/F | 6.8 |
| R→E | 47 | F/F | 8.0 |
| Y→A | 63 | F/F | 3.5 |
| Y→K | 63 | F/F | 10.2 |
| V→Q | 68 | F/F | 2.4 |
| N→A | 89 | F/F | 16.0 |
| N→W | 89 | F/F | 0.0 |
| Q→A | 95 | F/F | 51.1 |
| Q→W | 95 | F/F | 103.6 |
| R→A | 102 | F/F | 17.9 |
| F→Q | 111 | F/F | 17.9 |

Mutations F111Q and Y63A resulted in poor secretion.

### Example 25

### Combined Mutations in IL-17A

Mutations were made in a dimeric protein in which Subunit 1 contained the mutations identified in the first column in Table 7 in an IL-17A background, and Subunit 2 contained the mutations identified in the second column in an IL-17A background. The acid/base zipper approach described in Example 21 was used to produce dimers containing Subunit 1 and Subunit 2. Proteins were expressed in 293 cells and supernatants were collected and assayed. The ability of proteins to agonize in the assay described in Example 23 was evaluated and compared to a wildtype IL-17A/IL-17A dimer.

Another useful reference sequence for IL-17A is as follows and corresponds to SEQ ID NO:2 with the N-terminal glycine included:

(SEQ ID NO: 20)
GITIPRNPGCPNSEDKNFPRTVMVNLNIHNRNTNTNPKRSSDYYN
RSTSPWNLHRNEDPERYPSVIWEAKCRHLGCINADGNVDYHMNSV
PIQQEILVLRREPPHCPNSFRLEKILVSVGCTCVTPIVHHVA

Proteins were prepared using subunits having the sequences listed below and where mutations in IL-17A were identified according to the numbering of the reference sequence above (col. 2) and according to IL-17F numbering (col. 3):

TABLE 6

| | Mutation in | Mutation in | |
|---|---|---|---|
| | SEQ ID NO: 20 | SEQ ID NO: 12 | SEQ |
| Sequence | reference | reference | ID NO: |
| GITIPRNPGCPNSEDKNFPRTVMVNLNIHNRN | R46R | R47E | 21 |
| TNTNPKRSSDYYNESTSPWNLHRNEDPERYPS | | | |
| VIWEAKCRHLGCINADGNVDYHMNSVPIQQEI | | | |
| LVLRREPPHCPNSFRLEKILVSVGCTCVTPIV | | | |
| HHVA | | | |
| GITIPRNPGCPNSEDKNFPRTVMVNLNIHNRN | R46E, S64K | R47E, S65K | 22 |
| TNTNPKRSSDYYNESTSPWNLHRNEDPERYPK | | | |
| VIWEAKCRHLGCINADGNVDYHMNSVPIQQEI | | | |
| LVLRREPPHCPNSFRLEKILVSVGCTCVTPIV | | | |
| HHVA | | | |
| GITIPRNPGCPNSEDKNFPRTVMVNLNIHNRN | R46E, S64W | R47E, S65W | 23 |
| TNTNPKRSSDYYNESTSPWNLHRNEDPERYPW | | | |
| VIWEAKCRHLGCINADGNVDYHMNSVPIQQEI | | | |
| LVLRREPPHCPNSFRLEKILVSVGCTCVTPIV | | | |
| HHVA | | | |
| GITIPRNPGCPNSEDKNFPRTVMVNLNIHNRN | R46E, W67Q | R47E, W68Q | 24 |
| TNTNPKRSSDYYNESTSPWNLHRNEDPERYPS | | | |
| VIQEAKCRHLGCINADGNVDYHMNSVPIQQEI | | | |
| LVLRREPPHCPNSFRLEKILVSVGCTCVTPIV | | | |
| HHVA | | | |
| GITIPRNPGCPNSEDKNFPRTVMVNLNIHNRN | R46E, R101A | R47E, R102A | 25 |
| TNTNPKRSSDYYNESTSPWNLHRNEDPERYPS | | | |
| VIWEAKCRHLGCINADGNVDYHMNSVPIQQEI | | | |
| LVLRAEPPHCPNSFRLEKILVSVGCTCVTPIV | | | |
| HHVA | | | |
| GITIPRNPGCPNSEDKNFPRTVMVNLNIHNRN | N88A | N89A | 26 |
| TNTNPKRSSDYYNRSTSPWNLHRNEDPERYPS | | | |
| VIWEAKCRHLGCINADGNVDYHMASVPIQQEI | | | |
| LVLRREPPHCPNSFRLEKILVSVGCTCVTPIV | | | |
| HHVA | | | |
| GITIPRNPGCPNSEDKNFPRTVMVNLNIHNRN | N88A, | N89A, | 27 |
| TNTNPKRSSDYYNRSTSPWNLHRNEDPERYPS | trunc after | trunc after | |
| VIWEAKCRHLGCINADGNVDYHMASVPIQQEI | P126 | P127 | |
| LVLRREPPHCPNSFRLEKILVSVGCTCVTP | | | |

The C-terminal truncation was immediately before position 128 of SEQ ID NO:12 (position 126 of SEQ ID NO:2 or position 127 in SEQ ID NO:20), leaving proline at position 127 of SEQ ID NO:12 (position 125 of SEQ ID NO:2 or position 126 in SEQ ID NO:20).

TABLE 7

| | | % Activity |
|---|---|---|
| Subunit 1 | Subunit 2 | of Wildtype |
| R47E, S65K | WT | 17.0 |
| (SEQ ID NO: 22) | (SEQ ID NO: 20) | |
| R47E, W68Q | WT | 28.4 |
| (SEQ ID NO: 24) | (SEQ ID NO: 20) | |
| R47E, R102A | WT | 43.9 |
| (SEQ ID NO: 25) | (SEQ ID NO: 20) | |
| R47E | N89A | 34.2 |
| (SEQ ID NO: 21) | (SEQ ID NO: 26) | |
| R47E, S65K | N89A | 6.6 |
| (SEQ ID NO: 22) | (SEQ ID NO: 26) | |
| R47E, W68Q | N89A | ~0.0 |
| (SEQ ID NO: 24) | (SEQ ID NO: 26) | |
| R47E, R102A | N89A | ~0.0 |
| (SEQ ID NO: 25) | (SEQ ID NO: 26) | |
| R47E | N89A + C-terminal trunc. | ~0.0 |
| (SEQ ID NO: 21) | (SEQ ID NO: 27) | |
| R47E, S65K | N89A + C-terminal trunc. | ~0.0 |
| (SEQ ID NO: 22) | (SEQ ID NO: 27) | |
| R47E, W68Q | N89A + C-terminal trunc. | 1.3 |
| (SEQ ID NO: 24) | (SEQ ID NO: 27) | |
| R47E, R102A | N89A + C-terminal trunc. | ~0.0 |
| (SEQ ID NO: 25) | (SEQ ID NO: 27) | |

In addition, proteins with the following combinations of mutations (wt/N89A), (R47E/N89A), (R47E, S65K/N89A), (R47E, W68Q/N89A), (R47E, R102A/N89A) were observed to bind to IL-17RA in a plate binding assay.

### Example 26

Still other exemplary mutant sequences for other human IL-17 cytokines include:

TABLE 8

| | Mutation | |
|---|---|---|
| | identified by | |
| | IL-17F | SEQ |
| IL-17F: | numbering | ID NO: |
| RKIPKVGHTFFQKPESCPPVPGGSMKLDIGIINENQRVSMSR | R47E | 28 |
| NIESESTSPWNYTVTWDPNRYPSEVVQAQCRNLGCINAQGKE | | |
| DISMNSVPIQQETLVVRRKHQGCSVSFQLEKVLVTVGCTCVT | | |
| PVIHHVQ | | |
| RKIPKVGHTFFQKPESCPPVPGGSMKLDIGIINENQRVSMSR | R47E; S65K | 29 |
| NIESESTSPWNYTVTWDPNRYPKEVVQAQCRNLGCINAQGKE | | |
| DISMNSVPIQQETLVVRRKHQGCSVSFQLEKVLVTVGCTCVT | | |
| PVIHHVQ | | |
| RKIPKVGHTFFQKPESCPPVPGGSMKLDIGIINENQRVSMSR | R47E; S65W | 30 |
| NIESESTSPWNYTVTWDPNRYPWEVVQAQCRNLGCINAQGKE | | |
| DISMNSVPIQQETLVVRRKHQGCSVSFQLEKVLVTVGCTCVT | | |
| PVIHHVQ | | |
| RKIPKVGHTFFQKPESCPPVPGGSMKLDIGIINENQRVSMSR | R47E; V68Q | 31 |
| NIESESTSPWNYTVTWDPNRYPSEVQQAQCRNLGCINAQGKE | | |
| DISMNSVPIQQETLVVRRKHQGCSVSFQLEKVLVTVGCTCVT | | |
| PVIHHVQ | | |
| RKIPKVGHTFFQKPESCPPVPGGSMKLDIGIINENQRVSMSR | R47E; R102A | 32 |
| NIESESTSPWNYTVTWDPNRYPSEVVQAQCRNLGCINAQGKE | | |
| DISMNSVPIQQETLVVRAKHQGCSVSFQLEKVLVTVGCTCVT | | |
| PVIHHVQ | | |
| RKIPKVGHTFFQKPESCPPVPGGSMKLDIGIINENQRVSMSR | N89A | 33 |
| NIESRSTSPWNYTVTWDPNRYPSEVVQAQCRNLGCINAQGKE | | |
| DISMASVPIQQETLVVRRKHQGCSVSFQLEKVLVTVGCTCVT | | |
| PVIHHVQ | | |
| IL-17B: | | |
| RSPKSKRKGQGRPGPLAPGPHQVPLDLVSRMKPYARMEEYER | R47E | 34 |
| NIEEMVAQLRNSSELAQRKCEVNLQLWMSNKESLSPWGYSIN | | |
| HDPSRIPVDLPEARCLCLGCVNPFTMQEDRSMVSVPVFSQVP | | |
| VRRRLCPPPPRTGPCRQRAVMETIAVGCTCIF | | |
| RSPKSKRKGQGRPGPLAPGPHQVPLDLVSRMKPYARMEEYER | R47E; R102A | 35 |
| NIEEMVAQLRNSSELAQRKCEVNLQLWMSNKESLSPWGYSIN | | |
| HDPSRIPVDLPEARCLCLGCVNPFTMQEDRSMVSVPVFSQVP | | |
| VRRALCPPPPRTGPCRQRAVMETIAVGCTCIF | | |
| RSPKSKRKGQGRPGPLAPGPHQVPLDLVSRMKPYARMEEYER | V89A | 36 |
| NIEEMVAQLRNSSELAQRKCEVNLQLWMSNKRSLSPWGYSIN | | |
| HDPSRIPVDLPEARCLCLGCVNPFTMQEDRSMASVPVFSQVP | | |
| VRRRLCPPPPRTGPCRQRAVMETIAVGCTCIF | | |
| IL-17C: | | |
| HHDPSLRGHPHSHGTPHCYSAEELPLGQAPPHLLARGAKWGQ | R47E | 37 |
| ALPVALVSSLEAASHRGRHERPSATTQCPVLRPEEVLEADTH | | |
| QESISPWRYRVDTDEDRYPQKLAFAECLCRGCIDARTGRETA | | |
| ALNSVRLLQSLLVLRRRPCSRDGSGLPTPGAFAFHTEFIHVP | | |
| VGCTCVLPRSV | | |
| HHDPSLRGHPHSHGTPHCYSAEELPLGQAPPHLLARGAKWGQ | R47E; R102A | 38 |
| ALPVALVSSLEAASHRGRHERPSATTQCPVLRPEEVLEADTH | | |
| QESISPWRYRVDTDEDRYPQKLAFAECLCRGCIDARTGRETA | | |
| ALNSVRLLQSLLVLRARPCSRDGSGLPTPGAFAFHTEFIHVP | | |
| VGCTCVLPRSV | | |
| HHDPSLRGHPHSHGTPHCYSAEELPLGQAPPHLLARGAKWGQ | N89A | 39 |
| ALPVALVSSLEAASHRGRHERPSATTQCPVLRPEEVLEADTH | | |
| QRSISPWRYRVDTDEDRYPQKLAFAECLCRGCIDARTGRETA | | |
| ALASVRLLQSLLVLRRRPCSRDGSGLPTPGAFAFHTEFIHVP | | |
| VGCTCVLPRSV | | |
| IL-17D: | | |
| AGAPRAGRRPARPRGCADRPEELLEQLYGRLAAGVLSAFHHT | R47E | 40 |
| LQLGPREQARNASCPAGGRPADRRFRPPTNLESVSPWAYRIS | | |
| YDPARYPRYLPEAYCLCRGCLTGLFGEEDVRFRSAPVYMPTV | | |
| VLRRTPACAGGRSVYTEAYVTIPVGCTCVPEPEKDADSINSS | | |
| IDKQGAKLLLGPNDAPAGP | | |
| AGAPRAGRRPARPRGCADRPEELLEQLYGRLAAGVLSAFHHT | R47E; R102A | 41 |
| LQLGPREQARNASCPAGGRPADRRFRPPTNLESVSPWAYRIS | | |
| YDPARYPRYLPEAYCLCRGCLTGLFGEEDVRFRSAPVYMPTV | | |
| VLRATPACAGGRSVYTEAYVTIPVGCTCVPEPEKDADSINSS | | |
| IDKQGAKLLLGPNDAPAGP | | |
| AGAPRAGRRPARPRGCADRPEELLEQLYGRLAAGVLSAFHHT | R89A | 42 |
| LQLGPREQARNASCPAGGRPADRRFRPPTNLRSVSPWAYRIS | | |
| YDPARYPRYLPEAYCLCRGCLTGLFGEEDVRFASAPVYMPTV | | |
| VLRRTPACAGGRSVYTEAYVTIPVGCTCVPEPEKDADSINSS | | |
| IDKQGAKLLLGPNDAPAGP | | |
| IL-17E: | | |
| THTYSHWPSCCPSKGQDTSEELLRWSTVPVPPLEPARPNRHP | R47E | 43 |
| ESCRASEDGPLNSEAISPWRYELDRDLNRLPQDLYHARCLCP | | |
| HCVSLQTGSHMDPRGNSELLYHNQTVFYRRPCHGEKGTHKGY | | |
| CLERRLYRVSLACVCVRPRVMG | | |
| THTYSHWPSCCPSKGQDTSEELLRWSTVPVPPLEPARPNRHP | R47E; R102A | 44 |
| ESCRASEDGPLNSEAISPWRYELDRDLNRLPQDLYHARCLCP | | |
| HCVSLQTGSHMDPRGNSELLYHNQTVFYARPCHGEKGTHKGY | | |
| CLERRLYRVSLACVCVRPRVMG | | |
| THTYSHWPSCCPSKGQDTSEELLRWSTVPVPPLEPARPNRHP | N89A | 45 |
| ESCRASEDGPLNSRAISPWRYELDRDLNRLPQDLYHARCLCP | | |
| HCVSLQTGSHMDPRGASELLYHNQTVFYRRPCHGEKGTHKGY | | |
| CLERRLYRVSLACVCVRPRVMG | | |

### Example 27

A mutant single-chain IL-17A protein was evaluated in cell-based antagonism assay. Specifically, the mutant protein was a single-chain IL-17A in which one subunit included the R47E and S65K mutations (e.g., as shown above in SEQ ID NO:22) and the second subunit included the N89A mutation and the C-terminal truncation (as shown above in SEQ ID NO:27). The two subunits were joined by a linker of the (G₄S)₆ design. The protein also included a C-terminal histidine tag. The sequence of the protein was as follows:

(SEQ ID NO: 46)
GITIPRNPGCPNSEDKNFPRTVMVNLNIHNRNTNTNPKRSSDYYNES
TSPWNLHRNEDPERYPKVIWEAKCRHLGCINADGNVDYHMNSVPIQQ
EILVLRREPPHCPNSFRLEKILVSVGCTCVTPIVHHVASGGGGSGGG
GSGGGGSGGGGSGGGGSGGGGSGITIPRNPGCPNSEDKNFPRTVMVN
LNIHNRNTNTNPKRSSDYYNRSTSPWNLHRNEDPERYPSVIWEAKCR
HLGCINADGNVDYHMASVPIQQEILVLRREPPHCPNSFRLEKILVSV
GCTCVTPASHHHHHH

In a plate binding assay to the soluble extracellular domain of IL-17RA, the mutant protein was observed to bind with an affinity comparable to wild-type IL-17A (approximately within a factor of 4).

For the activity assay, MRC-5 human embryonic fibroblast cells were cultured in the wells of 96-well plates at a concentration of 1×10⁵ cells/well in DMEM+10% FBS. Mutant single-chain IL-17A proteins were added to the wells at a final concentration of 4-2400 nM. Wild-type IL-17A and TNF-α were added to the wells at final concentrations of 5 ng/mL and 2 ng/mL respectively. Cells were incubated at 37° C., 5% CO₂ for 48 hours. IL-6 concentration in the supernatants was then measured by ELISA using the Thermo Scientific Human IL-6 Screening Set (cat#ENESS0005). The results are shown in Table 9 below and demonstrate that this protein was able to antagonize IL-17A with an IC50 of about 10-15 nM.

TABLE 9

| sc17A-DN-conc nM | Normalized Signal |
|---|---|
| 0.00 | 1.00 |
| 3.84 | 0.96 |
| 19.20 | 0.50 |
| 96 | 0.28 |
| 480 | 0.27 |
| 2400 | 0.40 |

### REFERENCES

1. Mosmann T R, Coffman R L. Annu Rev Immunol. 1989; 7:145-173.
2. Harrington L E, et al. Nat. Immunol. 2005; 6:1123-1132.
3. Park H, et al. Nat. Immunol. 2005; 6:1133-1141.
4. Korn T, et al. IL-17 and Th17 Cells. Annu Rev Immunol. 2009; 27:485-517.
5. Weaver C T, Hatton R D, Mangan P R, Harrington L E. Annu Rev Immunol. 2007; 25:821-852.
6. Gaffen S L. Nat Rev Immunol. 2009
7. Hymowitz S G, et al. EMBO J. 2001; 20:5332-5341.
8. Wright J F, et al. J Biol. Chem. 2007; 282:13447-13455.
9. Toy D, et al. J. Immunol. 2006; 177:36-39.
10. Zheng Y, et al. Nat. Med. 2008; 14:282-289.
11. Ishigame H, et al. Immunity. 2009; 30:108-119.
12. O'Connor W, Jr, et al. Nat. Immunol. 2009; 10:603-609.
13. Novatchkova M, et al. Trends Biochem Sci. 2003; 28:226-229.
14. Kramer J M, et al. J. Immunol. 2007; 179:6379-6383.
15. Maitra A, et al. Proc Natl Acad Sci USA. 2007; 104:7506-7511.
16. Rochman Y, Spolski R, Leonard W J. Nat Rev Immunol. 2009; 9:480-490.
17. Claudio E, et al. J. Immunol. 2009; 182:1617-1630.
18. Chang S H, Park H, Dong C. J Biol. Chem. 2006; 281:35603-35607.
19. Qian Y, et al. Nat. Immunol. 2007; 8:247-256.
20. Kramer J M, et al. J. Immunol. 2006; 176:711-715.
21. Bazan J F. Proc Natl Acad Sci USA. 1990; 87:6934-6938.
22. Wright J F, et al. J. Immunol. 2008; 181:2799-2805.
23. Rickel E A, et al. J. Immunol. 2008; 181:4299-4310.
24. Shi Y, et al. J Biol. Chem. 2000; 275:19167-19176.
25. Lee J, et al. J Biol. Chem. 2001; 276:1660-1664.
26. Rong Z, et al. Cell Res. 2009; 19:208-215.
27. Wang X, Lupardus P, Laporte S L, Garcia K C. Annu Rev Immunol. 2009; 27:29-60.
28. He X L, Garcia K C. Science. 2004; 304:870-875.
29. Wehrman T, et al. Neuron. 2007; 53:25-38.
30. Gong Y, Cao P, Yu H J, Jiang T. Nature. 2008; 454:789-793.
31. Wiesmann C, et al. Cell. 1997; 91:695-704.
32. Wang X, Baloh R H, Milbrandt J, Garcia K C. Structure. 2006; 14:1083-1092.
33. Wiesmann C, Ultsch M H, Bass S H, de Vos A M. Nature. 1999; 401:184-188.
34. You Z, et al. Cancer Res. 2006; 66:175-183.
35. Li T S, et al. Cell Signal. 2006; 18:1287-1298.
36. LaPorte S L, et al. Cell. 2008; 132:259-272.
37. Dukkipati A, et al. Protein Expr Purif. 2008; 62:160-170.
38. Walter T S, et al. Structure. 2006; 14:1617-1622.
39. Barton W A, et al. Protein Sci. 2006; 15:2008-2013.
40. Leslie A G W. Joint CCP4+ESF-EAMCB Newsletter on Protein Crystallography. 1992; 26
41. The CCP4 suite: programs for protein crystallography. Acta Crystallogr D Biol Crystallogr. 1994; 50:760-763.
42. Strong M, et al. Proc Natl Acad Sci USA. 2006; 103:8060-8065.
43. McCoy A J, et al. Phaser crystallographic software. J Appl Crystallogr. 2007; 40:658-674.
44. de La Fortelle E, Bricogne G. Methods Enzymol. 1997; 276:472-494.
45. Emsley P, Cowtan K. Acta Crystallogr D Biol Crystallogr. 2004; 60:2126-2132.
46. Afonine P V, Grosse-Kunstleve R W, Adams P D. The Phenix refinement framework. CCP4 Newsletter. 2005; 42
47. Brunger A T. Version 1.2 of the Crystallography and NMR system. Nat. Protoc. 2007; 2:2728-2733.
48. Laskowski R A, Moss D S, Thornton J M. Main-chain bond lengths and bond angles in protein structures. J Mol. Biol. 1993; 231:1049-1067.
49. Hooft R W, Vriend G, Sander C, Abola E E. Errors in protein structures. Nature. 1996; 381:272.
50. DeLano W L. DeLano Scientific. San Carlos; CA, USA: 2002.

All references cited herein are hereby incorporated by reference in their entirety.

### EQUIVALENTS

The invention may be embodied in other specific forms without departing from the spirit or essential characteristics thereof. The foregoing embodiments are therefore to be considered in all respects illustrative rather than limiting on the invention described herein.

We claim:

1. An isolated protein comprising an antibody that binds to an IL-17 cytokine at an epitope in a region within:
a. about amino acids 21-41 of IL-17F (SEQ ID NO:12);
b. about amino acids 21-39 of IL-17A (SEQ ID NO:2);
c. about amino acids 44-65 of IL-17C (SEQ ID NO:6);
d. about amino acids 32-53 of IL-17D (SEQ ID NO:8);
e. about amino acids 27-49 of IL-17E (SEQ ID NO:10); or
f. about amino acids 32-53 of IL-17B (SEQ ID NO:4).

2. An isolated protein comprising an antibody that binds to an IL-17R at an epitope in a region within
about amino acids 22-36, 83-96, 118-147, 152-179, and/or 256-271 of IL-17RA (SEQ ID NO:14);
about amino acids 25-39, 86-100, 126-155, 160-187, and/or 254-269 of IL-17RB (SEQ ID NO:15); or
about amino acids 15-30, 70-84, 96-124, 129-156, and/or 227-237 of IL-17RC (SEQ ID NO:16).

3. An isolated protein comprising an isolated Interleukin 17 (IL-17) polypeptide comprising a sequence
that is at least 90% identical to IL-17A (SEQ ID NO:2), but less than 100% identical and one or more of amino acids selected from the group consisting of about 21-39, 40-76, 80-101, and 102-131 are mutated to any other amino acid or are deleted;
that is at least 90% identical to IL-17B (SEQ ID NO:4), but less than 100% identical and one or more of amino acids selected from the group consisting of 32-53, 66-105, 110-131, and 135-158 are mutated to any other amino acid or are deleted;
that is at least 90% identical to IL-17C (SEQ ID NO:6), but less than 100% identical and one or more of amino acids selected from the group consisting of about 44-65, 78-117, 121-143, and 153-179 are mutated to any other amino acid or are deleted;
that is at least 90% identical to IL-17D (SEQ ID NO:8), but less than 100% identical and one or more of amino acids selected from the group consisting of 32-53, 66-105, 110-131, and 134-163 are mutated to any other amino acid or are deleted;
that is at least 90% identical to IL-17E (SEQ ID NO:10), but less than 100% identical and one or more of amino acids selected from the group consisting of 27-49, 50-87, 93-114, and 120-148 are mutated to any other amino acid or are deleted; and
that is at least 90% identical to IL-17F (SEQ ID NO:12), but less than 100% identical and one or more of amino acids selected from the group consisting of about 21 to 41, 42-78, 82-103, and 104-133 are mutated to any other amino acid or are deleted.

4. An isolated protein comprising a first and second IL-17 subunit, wherein
the subunits differ from each another in amino acid sequence, and
the subunits form a dimer comprising a first face that is able to interact with a first IL-17 receptor subunit and a second face that has a reduced ability to interact with a second IL-17 receptor subunit relative to a corresponding natural IL-17 protein.

5. The protein of claim 4 wherein each subunit is at least 90% identical to a mature human IL-17 cytokine (SEQ ID NO:2, 4, 6, 8, 10, or 12) in the region corresponding to 1-127 of SEQ ID NO:12 and 1-125 of SEQ ID NO:2.

6. The protein of claim 5 wherein at least one subunit has between one and seven substitutions or deletions relative to a mature human IL-17 cytokine (SEQ ID NO:2, 4, 6, 8, 10, or 12) in the region corresponding to 1-127 of SEQ ID NO:12 and 1-125 of SEQ ID NO:2.

7. The protein of claim 5 wherein at least one subunit has between one and seven mutations relative to a mature human IL-17 cytokine (SEQ ID NO:2, 4, 6, 8, 10, or 12) in the region corresponding to 1-127 of SEQ ID NO:12 and 1-125 of SEQ ID NO:2 and a C-terminal truncation of residues corresponding to 128-133 of SEQ ID NO:12 or 126-131 of SEQ ID NO:2.

8. The protein of claim 5 wherein at least one subunit is identical to a mature human IL-17 cytokine (SEQ ID NO:2, 4, 6, 8, 10, or 12).

9. The protein of claim 5 wherein one subunit has between one and five mutations relative to a mature human IL-17 cytokine (SEQ ID NO:2, 4, 6, 8, 10, or 12) and the other subunit has a C-terminal deletion of at least four amino acids and optionally between one and five mutations relative to a mature human IL-17 cytokine (SEQ ID NO:2, 4, 6, 8, 10, or 12).

10. The protein of claim 5 wherein each subunit is at least 90% identical to the same human IL-17 cytokine as the other subunit.

11. The protein of claim 5 wherein one subunit is at least 90% identical to mature human IL-17A and the other subunit is at least 90% identical to mature IL-17F.

12. The protein of claim 4 wherein the second face of the dimer comprises at least one mutation in Site 1.

13. The protein of claim 4 wherein the second face of the dimer has between one and four mutations in Site 1.

14. The protein of claim 4 wherein the second face of the dimer comprises at least one mutation in Site 2.

15. The protein of claim 4 wherein the second face of the dimer has between one and four mutations in Site 2.

16. The protein of claim 4 wherein the second face of the dimer comprises at least one substitution or deletion in Site 3.

17. The protein of claim 4 wherein the second face of the dimer has between one and four substitutions in Site 3 and/or a C-terminal deletion of at least one amino acid.

18. The protein of claim 4 wherein the second face of the dimer has at least one substitution or deletion in at least two of the following sites: Site 1, Site 2, and Site 3.

19. The protein of claim 4 wherein the second face of the dimer has at least one substitution or deletion in each of the following sites: Site 1, Site 2, and Site 3.

20. The protein of claim 4 wherein the first subunit comprises a substitution at a position corresponding to R47 (according to numbering in SEQ ID NO:12).

21. The protein of claim 20 wherein the substitution at the position corresponding to R47 is to a non-basic residue.

22. The protein of claim 21 wherein the substitution at the position corresponding to R47 is to an acidic or hydrophobic residue.

23. The protein of claim 20 wherein the first subunit further comprises at least a second substitution at one or more positions corresponding to S65, V68, or R102 (according to numbering in SEQ ID NO:12) or S64, W67, or R101 (according to the numbering in SEQ ID NO:20).

24. The protein of claim 4 or 20 wherein the second subunit comprises a substitution at a position corresponding to N89 (according to numbering in SEQ ID NO:12).

25. The protein of claim 4 or 20 wherein the second subunit comprises a deletion or mutation of one or more of C-terminal residues corresponding to 128-133 (according to numbering in SEQ ID NO:12).

26. The protein of claim 4 or 20 wherein the second subunit is deleted for C-terminal residues corresponding to 128-133 (according to numbering in SEQ ID NO:12).

27. The protein of claim 4 wherein the first and second subunits are covalently attached.

28. The protein of claim 27 wherein the first and second subunits are components of the same polypeptide chain.

29. The protein of claim 4 that has an affinity for IL-17RA that is no more than 100-fold weaker than IL-17A/A, IL-17F/F, or IL-17A/F.

30. The protein of claim 4 that has an affinity for IL-17RC that is no more than 100-fold weaker than IL-17A/A, IL-17F/F, or IL-17A/F.

31. The protein of claim 4 wherein the first face does not contain any mutations relative to a mature human IL-17 cytokine (SEQ ID NO:2, 4, 6, 8, 10, or 12).

32. The protein of claim 4 wherein the first subunit comprises one or more mutations at positions corresponding to MET25 and LYS115 (according to numbering in SEQ ID NO:12) and/or the second subunit comprises one or more mutations at positions corresponding to ILE29, ILE31, TRP58, ASN61, TYR63, PRO64, SER65, GLU66, VAL100, ARG102, HIS104, VAL109, and PHE111 (according to numbering in SEQ ID NO:12).

33. The protein of claim 4 wherein the first subunit comprises one or more mutations at positions corresponding to GLN94, GLN95, GLU96, LYS115, and LEU117 (according to numbering in SEQ ID NO:12) and/or the second subunit comprises one or more mutations at positions corresponding to GLN36, ARG37, MET40, SER41, ASN43, GLU45, TYR54, VAL56, GLU66, VAL68, and VAL118 (according to numbering in SEQ ID NO:12).

34. The protein of claim 4 wherein the first subunit comprises one or more mutations at positions corresponding to LEU75, ILE86, SER87, ASN89, VAL91, VAL125, PRO127, VAL128, ILE129, HIS130, HIS131, and VAL132 (according to numbering in SEQ ID NO:12) and/or the second subunit comprises one or more mutations at positions corresponding to MET40, ARG42, ILE44, and ARG47 (according to numbering in SEQ ID NO:12).

35. An isolated protein comprising a first and second IL-17 subunit wherein:
each subunit is at least 90% identical to a human IL-17 polypeptide, and collectively the subunits includes at least two of the following substitutions or deletions relative to such human IL-17 polypeptide:
in the first subunit, a substitution at the position corresponding to R47 (according to numbering in SEQ ID NO:12),
in the first subunit, a substitution at the position corresponding to S65 (according to numbering in SEQ ID NO:12),
in the first subunit, a substitution at the position corresponding to W68 (according to numbering in SEQ ID NO:12),
in the first subunit, a substitution at the position corresponding to R102 (according to numbering in SEQ ID NO:12),
in the second subunit, a substitution at the position corresponding to N89 (according to numbering in SEQ ID NO:12),
in the second subunit, a substitution at the position corresponding to Q95 (according to numbering in SEQ ID NO:12), and
in the second subunit, one or more substitutions or deletions at positions corresponding to 127-132 (according to numbering in SEQ ID NO:12).

36. The protein of claim 35 wherein collectively the subunits includes at least two of the following substitutions or deletions relative to such human IL-17 polypeptide:
in the first subunit, a R47E, R47A, or R47D substitution at the position corresponding to R47 (according to numbering in SEQ ID NO:12),
in the first subunit, a S65K, S65R, or S65W substitution at the position corresponding to S65 (according to numbering in SEQ ID NO:12),
in the first subunit, a W68A, W68V, W68S, W68Q or W68N substitution at the position corresponding to W68 (according to numbering in SEQ ID NO:12),
in the first subunit, a R102A, R102V, R102S or R102T substitution at the position corresponding to R102 (according to numbering in SEQ ID NO:12),
in the second subunit, a N89A or N89V substitution at the position corresponding to N89 (according to numbering in SEQ ID NO:12),
in the second subunit, a Q95A or Q95W substitution at the position corresponding to Q95 (according to numbering in SEQ ID NO:12), and
in the second subunit, a deletion of at least positions corresponding to 128-132 (according to numbering in SEQ ID NO:12).

37. An isolated protein comprising a protein comprising (1) a first Interleukin-17 polypeptide and (2) a second Interleukin-17 polypeptide, wherein one or both of the IL-17 polypeptides are mutated forms of a human IL-17 cytokine and the first and second IL-17 polypeptides associate to form a dimer.

38. The isolated protein of claim 37 wherein the first and second IL-17 polypeptides are each at least 95% identical to a human IL-17 cytokine.

39. The isolated protein of claim 37 wherein the first and second IL-17 polypeptides are components of a single polypeptide chain.

40. The isolated protein of claim 37 wherein the protein further comprises an Fc domain or an albumin binding domain.

41. The isolated protein of claim 37 wherein the first and second polypeptides are operably linked by a coiled-coil domain or a leucine zipper.

42. The isolated protein of claim 37 wherein the first or second polypeptide comprises a sequence identical to a human IL-17 cytokine.

43. An isolated protein comprising one or more of the following polypeptide sequences selected from the group consisting of SEQ ID NO:21-46 or a sequence at least 95% identical to SEQ ID NO:21-46 but differing from a natural mature IL-17 cytokine.

44. A pharmaceutical composition comprising a protein according to any of claims 1-43.

45. A method of modulating an immune or inflammatory response in a subject, the method comprising:
administering a composition according to claim 44 to a subject in an amount effective to modulate the immune or inflammatory response in the subject.

46. A method of treating an IL-17 mediated disorder in a subject, the method comprising:
administering a composition according to claim 44 to a subject in an amount effective to modulate the immune or inflammatory response in the subject.

47. An isolated nucleic acid comprising one or more sequences encoding the protein of any of claims 1 to 44 or a polypeptide chain thereof.

48. A recombinant host cell comprising a recombinant nucleic acid containing one or more sequences encoding the protein of any of claims 1 to 44 or a polypeptide chain thereof.

49. A method of preparing a recombinant protein, the method comprising
culturing the host cell of claim 48 under conditions that permit expression of the recombinant protein, and
recovering the recombinant protein.

50. The method of claim 49 wherein the recombinant protein is purified from cell lysate or cell media and/or the method includes formulating the recombinant protein with one or more of an excipient, a stabilizer, and a buffer.


SEQUENCE LISTING (46 sequences)

SEQ ID NO: 1 - protein, 155 aa, Homo sapiens
MTPGKTSLVSLLLLLSLEAIVKAGITIPRNPGCPNSEDKNFPRTVMVNLNIHNRNTNTNPKRSSDYYNRSTSPWNLHRNEDPERYPSVIWEAKCRHLGCINADGNVDYHMNSVPIQQEILVLRREPPHCPNSFRLEKILVSVGCTCVTPIVHHVA

SEQ ID NO: 2 - protein, 131 aa, Homo sapiens
ITIPRNPGCPNSEDKNFPRTVMVNLNIHNRNTNTNPKRSSDYYNRSTSPWNLHRNEDPERYPSVIWEAKCRHLGCINADGNVDYHMNSVPIQQEILVLRREPPHCPNSFRLEKILVSVGCTCVTPIVHHVA

SEQ ID NO: 3 - protein, 180 aa, Homo sapiens
MDWPHNLLFLLTISIFLGLGQPRSPKSKRKGQGRPGPLAPGPHQVPLDLVSRMKPYARMEEYERNIEEMVAQLRNSSELAQRKCEVNLQLWMSNKRSLSPWGYSINHDPSRIPVDLPEARCLCLGCVNPFTMQEDRSMVSVPVFSQVPVRRRLCPPPPRTGPCRQRAVMETIAVGCTCIF

SEQ ID NO: 4 - protein, 158 aa, Homo sapiens
RSPKSKRKGQGRPGPLAPGPHQVPLDLVSRMKPYARMEEYERNIEEMVAQLRNSSELAQRKCEVNLQLWMSNKRSLSPWGYSINHDPSRIPVDLPEARCLCLGCVNPFTMQEDRSMVSVPVFSQVPVRRRLCPPPPRTGPCRQRAVMETIAVGCTCIF

SEQ ID NO: 5 - protein, 197 aa, Homo sapiens
MTLLPGLLFLTWLHTCLAHHDPSLRGHPHSHGTPHCYSAEELPLGQAPPHLLARGAKWGQALPVALVSSLEAASHRGRHERPSATTQCPVLRPEEVLEADTHQRSISPWRYRVDTDEDRYPQKLAFAECLCRGCIDARTGRETAALNSVRLLQSLLVLRRRPCSRDGSGLPTPGAFAFHTEFIHVPVGCTCVLPRSV

SEQ ID NO: 6 - protein, 179 aa, Homo sapiens
HHDPSLRGHPHSHGTPHCYSAEELPLGQAPPHLLARGAKWGQALPVALVSSLEAASHRGRHERPSATTQCPVLRPEEVLEADTHQRSISPWRYRVDTDEDRYPQKLAFAECLCRGCIDARTGRETAALNSVRLLQSLLVLRRRPCSRDGSGLPTPGAFAFHTEFIHVPVGCTCVLPRSV

SEQ ID NO: 7 - protein, 202 aa, Homo sapiens
MLVAGFLLALPPSWAAGAPRAGRRPARPRGCADRPEELLEQLYGRLAAGVLSAFHHTLQLGPREQARNASCPAGGRPADRRFRPPTNLRSVSPWAYRISYDPARYPRYLPEAYCLCRGCLTGLFGEEDVRFRSAPVYMPTVVLRRTPACAGGRSVYTEAYVTIPVGCTCVPEPEKDADSINSSIDKQGAKLLLGPNDAPAGP

SEQ ID NO: 8 - protein, 187 aa, Homo sapiens
AGAPRAGRRPARPRGCADRPEELLEQLYGRLAAGVLSAFHHTLQLGPREQARNASCPAGGRPADRRFRPPTNLRSVSPWAYRISYDPARYPRYLPEAYCLCRGCLTGLFGEEDVRFRSAPVYMPTVVLRRTPACAGGRSVYTEAYVTIPVGCTCVPEPEKDADSINSSIDKQGAKLLLGPNDAPAGP

SEQ ID NO: 9 - protein, 177 aa, Homo sapiens
MRERPRLGEDSSLISLFLQVVAFLAMVMGTHTYSHWPSCCPSKGQDTSEELLRWSTVPVPPLEPARPNRHPESCRASEDGPLNSRAISPWRYELDRDLNRLPQDLYHARCLCPHCVSLQTGSHMDPRGNSELLYHNQTVFYRRPCHGEKGTHKGYCLERRLYRVSLACVCVRPRVMG

SEQ ID NO: 10 - protein, 148 aa, Homo sapiens
THTYSHWPSCCPSKGQDTSEELLRWSTVPVPPLEPARPNRHPESCRASEDGPLNSRAISPWRYELDRDLNRLPQDLYHARCLCPHCVSLQTGSHMDPRGNSELLYHNQTVFYRRPCHGEKGTHKGYCLERRLYRVSLACVCVRPRVMG

SEQ ID NO: 11 - protein, 163 aa, Homo sapiens
MTVKTLHGPAMVKYLLLSILGLAFLSEAAARKIPKVGHTFFQKPESCPPVPGGSMKLDIGIINENQRVSMSRNIESRSTSPWNYTVTWDPNRYPSEVVQAQCRNLGCINAQGKEDISMNSVPIQQETLVVRRKHQGCSVSFQLEKVLVTVGCTCVTPVIHHVQ

SEQ ID NO: 12 - protein, 133 aa, Homo sapiens [sequence omitted]

SEQ ID NO: 13 - protein, 866 aa, Homo sapiens
MGAARSPPSAVPGPLLGLLLLLLGVLAPGGASLRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLWVYWFITGISILLVGSVILLIVCMTWRLAGPGSEKYSDDTKYTDGLPAADLIPPPLKPRKVWIIYSADHPLYVDVVLKFAQFLLTACGTEVALDLLEEQAISEAGVMTWVGRQKQEMVESNSKIIVLCSRGTRAKWQALLGRGAPVRLRCDHGKPVGDLFTAAMNMILPDFKRPACFGTYVVCYFSEVSCDGDVPDLFGAAPRYPLMDRFEEVYFRIQDLEMFQPGRMHRVGELSGDNYLRSPGGRQLRAALDRFRDWQVRCPDWFECENLYSADDQDAPSLDEEVFEEPLLPPGTGIVKRAPLVREPGSQACLAIDPLVGEEGGAAVAKLEPHLQPRGQPAPQPLHTLVLAAEEGALVAAVEPGPLADGAAVRLALAGEGEACPLLGSPGAGRNSVLFLPVDPEDSPLGSSTPMASPDLLPEDVREHLEGLMLSLFEQSLSCQAQGGCSRPAMVLTDPHTPYEEEQRQSVQSDQGYISRSSPQPPEGLTEMEEEEEEEQDPGKPALPLSPEDLESLRSLQRQLLFRQLQKNSGWDTMGSESEGPSA

SEQ ID NO: 14 - protein, 835 aa, Homo sapiens
SLRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLWVYWFITGISILLVGSVILLIVCMTWRLAGPGSEKYSDDTKYTDGLPAADLIPPPLKPRKVWIIYSADHPLYVDVVLKFAQFLLTACGTEVALDLLEEQAISEAGVMTWVGRQKQEMVESNSKIIVLCSRGTRAKWQALLGRGAPVRLRCDHGKPVGDLFTAAMNMILPDFKRPACFGTYVVCYFSEVSCDGDVPDLFGAAPRYPLMDRFEEVYFRIQDLEMFQPGRMHRVGELSGDNYLRSPGGRQLRAALDRFRDWQVRCPDWFECENLYSADDQDAPSLDEEVFEEPLLPPGTGIVKRAPLVREPGSQACLAIDPLVGEEGGAAVAKLEPHLQPRGQPAPQPLHTLVLAAEEGALVAAVEPGPLADGAAVRLALAGEGEACPLLGSPGAGRNSVLFLPVDPEDSPLGSSTPMASPDLLPEDVREHLEGLMLSLFEQSLSCQAQGGCSRPAMVLTDPHTPYEEEQRQSVQSDQGYISRSSPQPPEGLTEMEEEEEEEQDPGKPALPLSPEDLESLRSLQRQLLFRQLQKNSGWDTMGSESEGPSA

SEQ ID NO: 15 - protein, 502 aa, Homo sapiens
MSLVLLSLAALCRSAVPREPTVQCGSETGPSPEWMLQHDLIPGDLRDLRVEPVTTSVATGDYSILMNVSWVLRADASIRLLKATKICVTGKSNFQSYSCVRCNYTEAFQTQTRPSGGKWTFSYIGFPVELNTVYFIGAHNIPNANMNEDGPSMSVNFTSPGCLDHIMKYKKKCVKAGSLWDPNITACKKNEETVEVNFTTTPLGNRYMALIQHSTIIGFSQVFEPHQKKQTRASVVIPVTGDSEGATVQLTPYFPTCGSDCIRHKGTVVLCPQTGVPFPLDNNKSKPGGWLPLLLLSLLVATWVLVAGIYLMWRHERIKKTSFSTTTLLPPIKVLVVYPSEICFHHTICYFTEFLQNHCRSEVILEKWQKKKIAEMGPVQWLATQKKAADKVVFLLSNDVNSVCDGTCGKSEGSPSENSQDLFPLAFNLFCSDLRSQIHLHKYVVVYFREIDTKDDYNALSVCPKYHLMKDATAFCAELLHVKQQVSAGKRSQACHDGCCSL

SEQ ID NO: 16 - protein, 791 aa, Homo sapiens
MPVPWFLLSLALGRSPVVLSLERLVGPQDATHCSPVSLEPWGDEERLRVQFLAQQSLSLAPVTAATARTALSGLSGADGRREERGRGKSWVCLSLGGSGNTEPQKKGLSCRLWDSDILCLPGDIVPAPGPVLAPTHLQTELVLRCQKETDCDLCLRVAVHLAVHGHWEEPEDEEKFGGAADSGVEEPRNASLQAQVVLSFQAYPTARCVLLEVQVPAALVQFGQSVGSVVYDCFEAALGSEVRIWSYTQPRYEKELNHTQQLPDCRGLEVWNSIPSCWALPWLNVSADGDNVHLVLNVSEEQHFGLSLYWNQVQGPPKPRWHKNLTGPQIITLNHTDLVPCLCIQVWPLEPDSVRTNICPFREDPRAHQNLWQAARLQLLTLQSWLLDAPCSLPAEAALCWRAPGGDPCQPLVPPLSWENVTVDKVLEFPLLKGHPNLCVQVNSSEKLQLQECLWADSLGPLKDDVLLLETRGPQDNRSLCALEPSGCTSLPSKASTRAARLGEYLLQDLQSGQCLQLWDDDLGALWACPMDKYIHKRWALVWLACLLFAAALSLILLLKKDHAKGWLRLLKQDVRSGAAARGRAALLLYSADDSGFERLVGALASALCQLPLRVAVDLWSRRELSAQGPVAWFHAQRRQTLQEGGVVVLLFSPGAVALCSEWLQDGVSGPGAHGPHDAFRASLSCVLPDFLQGRAPGSYVGACFDRLLHPDAVPALFRTVPVFTLPSQLPDFLGALQQPRAPRSGRLQERAEQVSRALQPALDSYFHPPGTPAPGRGVGPGAGPGAGDGT

SEQ ID NO: 17 - protein, 195 aa, Homo sapiens
GITIPRNPGCPNSEDKNFPRTVMVNLNIHNRNTNTNPKRSSDYYNRSTSPWNLHRNEDPERYPSVIWEAKCRHLGCINADGNVDYHMNSVPIQQEILVLRREPPHCPNSFRLEKILVSVGCTCVTPIVHHVASGGGGSRGGLEVLFQGPEFGGSTTAPSAQLEKELQALEKENAQLEWELQALEKELAQHHHHHH

SEQ ID NO: 18 - protein, 195 aa, Homo sapiens
GITIPRNPGCPNSEDKNFPRTVMVNLNIHNRNTNTNPKRSSDYYNRSTSPWNLHRNEDPERYPSVIWEAKCRHLGCINADGNVDYHMNSVPIQQEILVLRREPPHCPNSFRLEKILVSVGCTCVTPIVHHVASGGGGSRGGLEVLFQGPEFGGSTTAPSAQLKKKLQALKKKNAQLKWKLQALKKKLAQHHHHHH

SEQ ID NO: 19 - protein, 302 aa, Homo sapiens [sequence omitted]

SEQ ID NO: 20 - protein, 132 aa, Homo sapiens
GITIPRNPGCPNSEDKNFPRTVMVNLNIHNRNTNTNPKRSSDYYNRSTSPWNLHRNEDPERYPSVIWEAKCRHLGCINADGNVDYHMNSVPIQQEILVLRREPPHCPNSFRLEKILVSVGCTCVTPIVHHVA

SEQ ID NO: 21 - protein, 132 aa, Homo sapiens
GITIPRNPGCPNSEDKNFPRTVMVNLNIHNRNTNTNPKRSSDYYNESTSPWNLHRNEDPERYPSVIWEAKCRHLGCINADGNVDYHMNSVPIQQEILVLRREPPHCPNSFRLEKILVSVGCTCVTPIVHHVA

SEQ ID NO: 22 - protein, 132 aa, Homo sapiens
GITIPRNPGCPNSEDKNFPRTVMVNLNIHNRNTNTNPKRSSDYYNESTSPWNLHRNEDPERYPKVIWEAKCRHLGCINADGNVDYHMNSVPIQQEILVLRREPPHCPNSFRLEKILVSVGCTCVTPIVHHVA

SEQ ID NO: 23 - protein, 132 aa, Homo sapiens
GITIPRNPGCPNSEDKNFPRTVMVNLNIHNRNTNTNPKRSSDYYNESTSPWNLHRNEDPERYPWVIWEAKCRHLGCINADGNVDYHMNSVPIQQEILVLRREPPHCPNSFRLEKILVSVGCTCVTPIVHHVA

SEQ ID NO: 24 - protein, 132 aa, Homo sapiens
GITIPRNPGCPNSEDKNFPRTVMVNLNIHNRNTNTNPKRSSDYYNESTSPWNLHRNEDPERYPSVIQEAKCRHLGCINADGNVDYHMNSVPIQQEILVLRREPPHCPNSFRLEKILVSVGCTCVTPIVHHVA

SEQ ID NO: 25 - protein, 132 aa, Homo sapiens
GITIPRNPGCPNSEDKNFPRTVMVNLNIHNRNTNTNPKRSSDYYNESTSPWNLHRNEDPERYPSVIWEAKCRHLGCINADGNVDYHMNSVPIQQEILVLRAEPPHCPNSFRLEKILVSVGCTCVTPIVHHVA

SEQ ID NO: 26 - protein, 132 aa, Homo sapiens
GITIPRNPGCPNSEDKNFPRTVMVNLNIHNRNTNTNPKRSSDYYNRSTSPWNLHRNEDPERYPSVIWEAKCRHLGCINADGNVDYHMASVPIQQEILVLRREPPHCPNSFRLEKILVSVGCTCVTPIVHHVA

SEQ ID NO: 27 - protein, 126 aa, Homo sapiens
GITIPRNPGCPNSEDKNFPRTVMVNLNIHNRNTNTNPKRSSDYYNRSTSPWNLHRNEDPERYPSVIWEAKCRHLGCINADGNVDYHMASVPIQQEILVLRREPPHCPNSFRLEKILVSVGCTCVTP

SEQ ID NO: 28 - protein, 133 aa, Homo sapiens
RKIPKVGHTFFQKPESCPPVPGGSMKLDIGIINENQRVSMSRNIESESTSPWNYTVTWDPNRYPSEVVQAQCRNLGCINAQGKEDISMNSVPIQQETLVVRRKHQGCSVSFQLEKVLVTVGCTCVTPVIHHVQ

SEQ ID NO: 29 - protein, 132 aa, Homo sapiens
RKIPKVGHTFFQKPESCPPVPGGSMKLDIGIINENQRVSMSRNIESESTSPWNYTVTWDPNRYPKEVVQAQCRNLGCINAQGKEDISMNSVPIQQETLVVRRKHQGCSVSFQLEKVLVTVGCTCVTPVIHHV

SEQ ID NO: 30 - protein, 133 aa, Homo sapiens
RKIPKVGHTFFQKPESCPPVPGGSMKLDIGIINENQRVSMSRNIESESTSPWNYTVTWDPNRYPWEVVQAQCRNLGCINAQGKEDISMNSVPIQQETLVVRRKHQGCSVSFQLEKVLVTVGCTCVTPVIHHVQ

SEQ ID NO: 31 - protein, 133 aa, Homo sapiens
RKIPKVGHTFFQKPESCPPVPGGSMKLDIGIINENQRVSMSRNIESESTSPWNYTVTWDPNRYPSEVQQAQCRNLGCINAQGKEDISMNSVPIQQETLVVRRKHQGCSVSFQLEKVLVTVGCTCVTPVIHHVQ

SEQ ID NO: 32 - protein, 133 aa, Homo sapiens
RKIPKVGHTFFQKPESCPPVPGGSMKLDIGIINENQRVSMSRNIESESTSPWNYTVTWDPNRYPSEVVQAQCRNLGCINAQGKEDISMNSVPIQQETLVVRAKHQGCSVSFQLEKVLVTVGCTCVTPVIHHVQ

SEQ ID NO: 33 - protein, 133 aa, Homo sapiens; identical to SEQ ID NO: 32

SEQ ID NO: 34 - protein, 158 aa, Homo sapiens
RSPKSKRKGQGRPGPLAPGPHQVPLDLVSRMKPYARMEEYERNIEEMVAQLRNSSELAQRKCEVNLQLWMSNKESLSPWGYSINHDPSRIPVDLPEARCLCLGCVNPFTMQEDRSMVSVPVFSQVPVRRRLCPPPPRTGPCRQRAVMETIAVGCTCIF

SEQ ID NO: 35 - protein, 158 aa, Homo sapiens
RSPKSKRKGQGRPGPLAPGPHQVPLDLVSRMKPYARMEEYERNIEEMVAQLRNSSELAQRKCEVNLQLWMSNKESLSPWGYSINHDPSRIPVDLPEARCLCLGCVNPFTMQEDRSMVSVPVFSQVPVRRALCPPPPRTGPCRQRAVMETIAVGCTCIF

SEQ ID NO: 36 - protein, 158 aa, Homo sapiens
RSPKSKRKGQGRPGPLAPGPHQVPLDLVSRMKPYARMEEYERNIEEMVAQLRNSSELAQRKCEVNLQLWMSNKRSLSPWGYSINHDPSRIPVDLPEARCLCLGCVNPFTMQEDRSMASVPVFSQVPVRRRLCPPPPRTGPCRQRAVMETIAVGCTCIF

SEQ ID NO: 37 - protein, 179 aa, Homo sapiens
HHDPSLRGHPHSHGTPHCYSAEELPLGQAPPHLLARGAKWGQALPVALVSSLEAASHRGRHERPSATTQCPVLRPEEVLEADTHQESISPWRYRVDTDEDRYPQKLAFAECLCRGCIDARTGRETAALNSVRLLQSLLVLRRRPCSRDGSGLPTPGAFAFHTEFIHVPVGCTCVLPRSV

SEQ ID NO: 38 - protein, 179 aa, Homo sapiens
HHDPSLRGHPHSHGTPHCYSAEELPLGQAPPHLLARGAKWGQALPVALVSSLEAASHRGRHERPSATTQCPVLRPEEVLEADTHQESISPWRYRVDTDEDRYPQKLAFAECLCRGCIDARTGRETAALNSVRLLQSLLVLRARPCSRDGSGLPTPGAFAFHTEFIHVPVGCTCVLPRSV

SEQ ID NO: 39 - protein, 179 aa, Homo sapiens
HHDPSLRGHPHSHGTPHCYSAEELPLGQAPPHLLARGAKWGQALPVALVSSLEAASHRGRHERPSATTQCPVLRPEEVLEADTHQRSISPWRYRVDTDEDRYPQKLAFAECLCRGCIDARTGRETAALASVRLLQSLLVLRRRPCSRDGSGLPTPGAFAFHTEFIHVPVGCTCVLPRSV

SEQ ID NO: 40 - protein, 187 aa, Homo sapiens
AGAPRAGRRPARPRGCADRPEELLEQLYGRLAAGVLSAFHHTLQLGPREQARNASCPAGGRPADRRFRPPTNLESVSPWAYRISYDPARYPRYLPEAYCLCRGCLTGLFGEEDVRFRSAPVYMPTVVLRRTPACAGGRSVYTEAYVTIPVGCTCVPEPEKDADSINSSIDKQGAKLLLGPNDAPAGP

SEQ ID NO: 41 - protein, 187 aa, Homo sapiens
AGAPRAGRRPARPRGCADRPEELLEQLYGRLAAGVLSAFHHTLQLGPREQARNASCPAGGRPADRRFRPPTNLESVSPWAYRISYDPARYPRYLPEAYCLCRGCLTGLFGEEDVRFRSAPVYMPTVVLRATPACAGGRSVYTEAYVTIPVGCTCVPEPEKDADSINSSIDKQGAKLLLGPNDAPAGP

SEQ ID NO: 42 - protein, 187 aa, Homo sapiens
AGAPRAGRRPARPRGCADRPEELLEQLYGRLAAGVLSAFHHTLQLGPREQARNASCPAGGRPADRRFRPPTNLRSVSPWAYRISYDPARYPRYLPEAYCLCRGCLTGLFGEEDVRFASAPVYMPTVVLRRTPACAGGRSVYTEAYVTIPVGCTCVPEPEKDADSINSSIDKQGAKLLLGPNDAPAGP

SEQ ID NO: 43 - protein, 148 aa, Homo sapiens
THTYSHWPSCCPSKGQDTSEELLRWSTVPVPPLEPARPNRHPESCRASEDGPLNSEAISPWRYELDRDLNRLPQDLYHARCLCPHCVSLQTGSHMDPRGNSELLYHNQTVFYRRPCHGEKGTHKGYCLERRLYRVSLACVCVRPRVMG

SEQ ID NO: 44 - protein, 148 aa, Homo sapiens
THTYSHWPSCCPSKGQDTSEELLRWSTVPVPPLEPARPNRHPESCRASEDGPLNSEAISPWRYELDRDLNRLPQDLYHARCLCPHCVSLQTGSHMDPRGNSELLYHNQTVFYARPCHGEKGTHKGYCLERRLYRVSLACVCVRPRVMG

SEQ ID NO: 45 - protein, 148 aa, Homo sapiens
THTYSHWPSCCPSKGQDTSEELLRWSTVPVPPLEPARPNRHPESCRASEDGPLNSRAISPWRYELDRDLNRLPQDLYHARCLCPHCVSLQTGSHMDPRGASELLYHNQTVFYRRPCHGEKGTHKGYCLERRLYRVSLACVCVRPRVMG

SEQ ID NO: 46 - protein, 297 aa, Homo sapiens
GITIPRNPGCPNSEDKNFPRTVMVNLNIHNRNTNTNPKRSSDYYNESTSPWNLHRNEDPERYPKVIWEAKCRHLGCINADGNVDYHMNSVPIQQEILVLRREPPHCPNSFRLEKILVSVGCTCVTPIVHHVASGGGGSGGGGSGGGGSGGGGSGGGGSGGGGSGITIPRNPGCPNSEDKNFPRTVMVNLNIHNRNTNTNPKRSSDYYNRSTSPWNLHRNEDPERYPSVIWEAKCRHLGCINADGNVDYHMASVPIQQEILVLRREPPHCPNSFRLEKILVSVGCTCVTPASHHHHHH